(19) United States
(12) Patent Application Publication
(10) Pub. No.: US 2020/0157537 A1
(43) Pub. Date: May 21, 2020

(54) Modulating RNA Interactions with Polycomb Repressive Complex 1 (PRC1)

(71) Applicant: The General Hospital Corporation, Boston, MA (US)

(72) Inventors: Jeannie T. Lee, Boston, MA (US); Michael Rosenberg, Brookline, MA (US); Roy Blum, Boston, MA (US)

(21) Appl. No.: 16/664,527

(22) Filed: Oct. 25, 2019

Related U.S. Application Data
(60) Provisional application No. 62/750,503, filed on Oct. 25, 2018.

(51) Int. Cl. C12N 15/113; A61P 37/06

(52) U.S. Cl. CPC C12N 15/113; C12N 2310/3233; C12N 2310/122; C12N 2310/11; C12N 2310/14; C12N 2310/3231; C12N 2310/321; C12N 2310/322; C12N 2310/3181; C12N 2310/315; C12N 2310/316; C12N 2310/313; C12N 2310/314; C12N 2310/311; A61P 37/06

(57) ABSTRACT

This invention relates to polycomb-associated RNAs, libraries and fragments of those RNAs, inhibitory nucleic acids and methods and compositions for targeting RNAs, and methods of use thereof.

### CLAIM OF PRIORITY

This application claims the benefit of U.S. Provisional Patent Application Ser. No. 62/750,503, filed on Oct. 25, 2018. The entire contents of the foregoing are hereby incorporated by reference.

### FEDERALLY SPONSORED RESEARCH OR DEVELOPMENT

This invention was made with Government support under Grant No. GM090278 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Oct. 25, 2019, is named SequenceListing.txt and is 67.2 MB in size.

### TECHNICAL FIELD

This invention relates to methods of modulating RNA interactions with Polycomb Repressive Complex 1 (PRC1) using inhibitory nucleic acids that bind RNAs and inhibit the PRC1-RNA interaction, to modulate gene expression.

### BACKGROUND

Polycomb group proteins play critically important roles in stem cell biology and mammalian development (Simon and Kingston, 2013). Polycomb proteins exist in at least two multi-subunit complexes, including Polycomb repressive complex 1 (PRC1) and Polycomb repressive complex 2 (PRC2). While PRC2 trimethylates histone H3 at lysine 27 (H3K27me3), PRC1 ubiquitylates histone H2A on lysine 119 (H2AK119Ub) through its RING-finger catalytic subunit, RING1a/1b, and compacts chromatin. Unlike PRC2, PRC1 has a heterogeneous composition in mammals. The “canonical” form of PRC1 is defined by inclusion of the chromobox homolog protein, CBX, which binds the H3K27me3 mark and is thereby partially dependent on PRC2 for chromatin binding. Canonical PRC1 has been associated with chromatin compaction through the CBX subunit (Grau et al., 2011). By contrast, the “noncanonical” form contains RING1 and YY1 Binding Protein (RYBP) and is associated predominantly with ubiquitylation of H2AK119 through the RING1 subunit. Noncanonical PRC1 binds chromatin independently of PRC2 and possibly helps direct PRC2 to chromatin through its H2AK119Ub mark (Aranda et al., 2015). In addition, PRC1 contains several other subunits, including Polycomb group RING finger protein 4 (PCGF4) (BMI1)/PCGF2 (MEL18); it also includes the polyhomeotic homolog (PHC) in the canonical (CBX) form, and PCGF1,2,4,5, and 6 in the non-canonical (RYBP) form. Together, PRC1 and PRC2 bind and regulate expression from thousands of genes in mammals (Blackledge et al., 2015).

### SUMMARY

The studies described herein demonstrated that PRC1 binds both noncoding RNA and coding RNA at identifieable sequence motifs, and that these motifs can be targeted to alter gene expression. The PRC1-interacting transcriptome includes antisense, intergenic, and promoter-associated transcripts, as well as many unannotated RNAs. A large number of transcripts occur within imprinted regions, oncogene and tumor suppressor loci, and stem-cell-related bivalent domains. Further evidence is provided that inhibitory oligonucleotides that specifically bind to these PRC1-interacting RNAs can successfully modulate gene expression in a variety of separate and independent examples, presumably by inhibiting PRC1-associated effects. PRC1 binding sites can be classified into several groups, including (i) 3′ untranslated region [3′ UTR], (ii) promoter-associated, (iii) gene body, (iv) antisense, and (v) intergenic. Inhibiting the PRC1-RNA interactions can lead to either activation or repression, depending on context.

In another aspect the invention features an inhibitory nucleic acid that specifically binds to, or is complementary to a region of an RNA comprising a motif as described herein that is known to bind to Polycomb repressive complex 1 (PRC1), wherein the sequence of the region is selected from the group consisting of SEQ ID NOs:1 to 5893 (human), 5894 to 17415 (human), and 17416 to 36368 (mouse), which are identical to those as set forth in Tables 1-3 of WO 2016/149455. Without being bound by a theory of invention, these inhibitory nucleic acids are able to interfere with the binding of and function of PRC1, by preventing recruitment of PRC1 to a specific chromosomal locus. For example, data herein shows that a single administration of inhibitory nucleic acids designed to specifically bind a RNA can alter expression of a gene associated with the RNA. Data provided herein also indicate that putative ncRNA binding sites for PRC1 show no conserved primary sequence motif, making it possible to design specific inhibitory nucleic acids that will interfere with PRC1 interaction with a single ncRNA, without generally disrupting PRC1 interactions with other ncRNAs. Further, data provided herein support that RNA can recruit PRC1 in a cis fashion, repressing gene expression at or near the specific chromosomal locus from which the RNA was transcribed, thus making it possible to design inhibitory nucleic acids that inhibit the function of PRC1 and increase the expression of a specific target gene.

In some embodiments, the inhibitory nucleic acid is provided for use in a method of modulating expression of a “gene targeted by the PRC1-binding RNA” (e.g., an intersecting or nearby gene, as set forth in Tables 1-3 of WO 2016/149455), meaning a gene whose expression is regulated by the PRC1-binding RNA. The term “PRC1-binding RNA” or “RNA that binds PRC1” is used interchangeably with “PRC1-associated RNA” and “PRC1-interacting RNA”, and refers to an RNA transcript or a region thereof (e.g., a Peak as described below) that binds the PRC1 complex, directly or indirectly. Such binding may be determined by dCLIP-SEQ techniques described herein using a component of the PRC1 complex, e.g., PRC1 itself. SEQ ID NOs: 1 to 5893 represent human RNA sequences containing portions that have been experimentally determined to bind PRC1 using the dCLIP-seq method described in WO 2016/149455; SEQ ID NOs: 17416 to 36368 represent murine RNA sequences containing portions that have been experimentally determined to bind PRC1 using the dCLIP-seq method; and SEQ ID NOs: 5894 to 17415 represent or human RNA sequences corresponding to the murine RNA sequences.

Such methods of modulating gene expression may be carried out in vitro, ex vivo, or in vivo. Tables 1-3 display genes targeted by the PRC1-binding RNA; the SEQ ID NOS: of the PRC1-associated RNA are set forth in the same row as the gene name. In some embodiments, the inhibitory nucleic acid is provided for use in a method of treating disease, e.g. a disease category as described herein. The treatment may involve modulating expression (either up or down) of a gene targeted by the PRC1-binding RNA, preferably upregulating gene expression. The inhibitory nucleic acid may be formulated as a sterile composition for parenteral administration. It is understood that any reference to uses of compounds throughout the description contemplates use of the compound in preparation of a pharmaceutical composition or medicament for use in the treatment of a disease. Thus, as one nonlimiting example, this aspect of the invention includes use of such inhibitory nucleic acids in the preparation of a medicament for use in the treatment of disease, wherein the treatment involves upregulating expression of a gene targeted by the PRC1-binding RNA.

Diseases, disorders or conditions that may be treated according to the invention include cardiovascular, metabolic, inflammatory, bone, neurological or neurodegenerative, pulmonary, hepatic, kidney, urogenital, bone, cancer, and/or protein deficiency disorders.

In a related aspect, the invention features a process of preparing an inhibitory nucleic acid that modulates gene expression, the process comprising the step of synthesizing an inhibitory nucleic acid of between 5 and 40 bases in length, or about 8 to 40, or about 5 to 50 bases in length, optionally single stranded, that specifically binds, or is complementary to, a motif as described herein within an RNA sequence that has been identified as binding to PRC1, optionally an RNA of any of Tables 1-3 of WO 2016/149455 or any one of SEQ ID NOs: 1 to 5893, or 5894 to 17415, or 17416 to 36368. This aspect of the invention may further comprise the step of identifying the RNA sequence as binding to PRC1, optionally through the dCLIP-seq method described herein.

In a further aspect of the present invention a process of preparing an inhibitory nucleic acid that specifically binds to an RNA that binds to Polycomb repressive complex 1 (PRC1) is provided, the process comprising the step of designing and/or synthesizing an inhibitory nucleic acid of between 5 and 40 bases in length, or about 8 to 40, or about 5 to 50 bases in length, optionally single stranded, that specifically binds to a motif within an RNA sequence that binds to PRC1, optionally an RNA of any of Tables 1-3 of WO 2016/149455 or any one of SEQ ID NOs: 1 to 5893, or 5894 to 17415, or 17416 to 36368.

In some embodiments prior to synthesizing the inhibitory nucleic acid the process further comprises identifying an RNA that binds to PRC1.

In some embodiments the RNA has been identified by a method involving identifying an RNA that binds to PRC1.

In some embodiments the inhibitory nucleic acid is at least 80% complementary to a contiguous sequence of between 5 and 40 bases, or about 8 to 40, or about 5 to 50 bases comprising said motif in said RNA sequence that binds to PRC1. In some embodiments the sequence of the designed and/or synthesized inhibitory nucleic acid is based on a said motif in an RNA sequence that binds to PRC1, or a portion thereof, said portion having a length of from 5 to 40 contiguous base pairs, or about 8 to 40 bases, or about 5 to 50 bases.

In some embodiments the sequence of the designed and/or synthesized inhibitory nucleic acid is based on a nucleic acid sequence that is complementary to said motif in an RNA sequence that binds to PRC1, or is complementary to a portion thereof, said portion having a length of from 5 to 40 contiguous base pairs, or about 8 to 40 base pairs, or about 5 to 50 base pairs.

The designed and/or synthesized inhibitory nucleic acid may be at least 80% complementary to (optionally one of at least 90%, 95%, 96%, 97%, 98%, 99% or 100% complementary to) the portion of the RNA sequence to which it binds or targets, or is intended to bind or target. In some embodiments it may contain 1, 2 or 3 base mismatches compared to the portion of the target RNA sequence or its complement respectively. In some embodiments it may have up to 3 mismatches over 15 bases, or up to 2 mismatches over 10 bases.

The inhibitory nucleic acid or portion of RNA sequence that binds to PRC1 may have a length of one of at least 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases. Where the inhibitory nucleic acid is based on an RNA motif sequence that binds to a PRC1, a nucleic acid sequence that is complementary to said RNA motif sequence that binds to PRC1 or a portion of such a sequence, it may be based on information about that sequence, e.g. sequence information available in written or electronic form, which may include sequence information contained in publicly available scientific publications or sequence databases.

In some embodiments, the isolated single stranded oligonucleotide is of 5 to 40 nucleotides in length and has a region of complementarity that is complementary with at least 5, 6, 7, 8, 9, or 10 contiguous nucleotides of a motif within the PRC1-binding RNA that inhibits expression of the target gene, e.g., as described in WO 2016/149455, wherein the oligonucleotide is complementary to and binds specifically within a motif in a PRC1-binding region of the PRC1-binding RNA and interferes with binding of PRC1 to the PRC1-binding region without inducing degradation of the PRC1-binding RNA (e.g., wherein the PRC1-binding region has a nucleotide sequence identified as a motif as described herein), and without interfering with binding of PRC2 to a PRC2-binding region of the RNA (as described in WO 2012/087983 or WO 2012/065143, wherein the PRC2-binding region has a nucleotide sequence protected from nucleases during an RNA immunoprecipitation procedure using an antibody directed against PRC2), optionally wherein the PRC1-binding RNA is transcribed from a sequence of the chromosomal locus of the target gene, and optionally wherein a decrease in recruitment of PRC1 to the target gene in the cell following delivery of the single stranded oligonucleotide to the cell, compared with an appropriate control cell to which the single stranded oligonucleotide has not been delivered, indicates effectiveness of the single stranded oligonucleotide.

Where the design and/or synthesis involves design and/or synthesis of a sequence that is complementary to a nucleic acid described by such sequence information the skilled person is readily able to determine the complementary sequence, e.g. through understanding of Watson-Crick base pairing rules which form part of the common general knowledge in the field.

In the methods described above the RNA that binds to PRC1 may be, or have been, identified, or obtained, by a method that involves identifying RNA that binds to PRC1, e.g., as described herein or in WO 2016/149455.

In one embodiment the method involves the dCLIP-Seq method described herein and in of WO 2016/149455.

In accordance with the above, in some embodiments the RNA that binds to PRC1 may be one that is known to bind PRC1, e.g. information about the sequence of the RNA and/or its ability to bind PRC1 is available to the public in written or electronic form allowing the design and/or synthesis of the inhibitory nucleic acid to be based on that information. As such, an RNA that binds to PRC1 may be selected from known sequence information and used to inform the design and/or synthesis of the inhibitory nucleic acid.

In other embodiments the RNA that binds to PRC1 may be identified as one that binds PRC1 as part of the method of design and/or synthesis.

In preferred embodiments design and/or synthesis of an inhibitory nucleic acid involves manufacture of a nucleic acid from starting materials by techniques known to those of skill in the art, where the synthesis may be based on a sequence of an RNA (or portion thereof) that has been selected as known to bind to Polycomb repressive complex 2.

Methods of design and/or synthesis of an inhibitory nucleic acid may involve one or more of the steps of:

Identifying and/or selecting a portion of an RNA sequence that binds to PRC1 (e.g., as shown in Tables 1-3 of WO 2016/149455 or as set forth in SEQ ID NOs:1 to 5893 (human), 5894 to 17415 (human), and 17416 to 36368 (mouse));

Designing a nucleic acid sequence having a desired degree of sequence identity or complementarity to a sequenc comprising a motif within an RNA sequence that binds to PRC1 or a portion thereof;

Synthesizing a nucleic acid to the designed sequence;

Mixing the synthesized nucleic acid with at least one pharmaceutically acceptable diluent, carrier or excipient to form a pharmaceutical composition or medicament.

Inhibitory nucleic acids so designed and/or synthesized may be useful in method of modulating gene expression as described herein.

As such, the process of preparing an inhibitory nucleic acid may be a process that is for use in the manufacture of a pharmaceutical composition or medicament for use in the treatment of disease, optionally wherein the treatment involves modulating expression of a gene targeted by the RNA binds to PRC1.

Methods for isolating RNA sequences that interact with a selected protein, e.g., with chromatin complexes, in a cell are further described in WO 2016/149455.

In yet another aspect, the invention features methods for increasing expression of a tumor suppressor in a mammal, e.g. human, in need thereof. The methods include administering to said mammal an inhibitory nucleic acid that specifically binds, or is complementary, to a sequence comprising a motif within a human PRC1-interacting RNA corresponding to a tumor suppressor locus of any of Tables 1-3 of WO 2016/149455or a human RNA corresponding to an imprinted gene of any of Tables 1-3 of WO 2016/149455 or as set forth in SEQ ID NOs:1 to 5893 (human), 5894 to 17415 (human), and 17416 to 36368 (mouse), or a related naturally occurring RNA that is othologous or at least 90%, (e.g., 91%, 92%, 93%, 94%, 95%, 96%, 97%, 98%%, or 100%) identical over at least 15 (e.g., at least 20, 21, 25, 30, 100) nucoleobases thereof, in an amount effective to increase expression of the tumor suppressor or growth suppressing gene. It is understood that one method of determining human orthologous RNA that corresponds to murine RNA is to identify a corresponding human sequence at least 90% identical (e.g., 91%, 92%, 93%, 94%, 95%, 96%, 97%, 98%, 99%, or 100% identical) to at least 15 nucleobases of the murine sequence (or at least 20, 21, 25, 30, 40, 50, 60, 70, 80, 90 or 100 nucleobases).

In an additional aspect, the invention provides methods for inhibiting or suppressing tumor growth in a mammal, e.g. human, with cancer, comprising administering to said mammal an inhibitory nucleic acid that specifically binds, or is complementary, to a sequence comorising a motif within a human PRC1-interacting RNA corresponding to a tumor suppressor locus of any of Tables 1-3 of WO 2016/149455, or a human RNA corresponding to an imprinted gene of any of Tables 1-3 of WO 2016/149455 or as set forth in SEQ ID NOs:1 to 5893 (human), 5894 to 17415 (human), and 17416 to 36368 (mouse), or a related naturally-occurring RNA that is orthologous or at least 90%, (e.g., 91%, 92%, 93%, 94%, 95%, 96%, 97%, 98%, 99% or 100%) identical over at least 15 (e.g., at least 20, 21, 25, 30, 50, 70, 100) nucleobases thereof, in an amount effective to suppress or inhibit tumor growth.

In another aspect, the invention features methods for treating a mammal, e.g., a human, with cancer comprising administering to said mammal an inhibitory nucleic acid that specifically binds, or is complementary, to a sequence comprising a motif within a human RNA corresponding to a tumor suppressor locus of any of Tables 1-3 of WO 2016/149455, or a human RNA corresponding to an imprinted gene of Tables 1-3 of WO 2016/149455, or as set forth in SEQ ID NOs:1 to 5893 (human), 5894 to 17415 (human), and 17416 to 36368 (mouse), or a related naturally occurring RNA that is orthologous or at least 90% (e.g.,91%, 92%, 93%, 94%, 95%, 96%, 97%, 98%, 99%, or 100%) identical over at least 15 (e.g., at least 20, 21, 25, 30, 50, 70, 100) nucleobases thereof, in a therapeutically effective amount.

Also provided herein are inhibitory nucleic acids that specifically bind, or are complementary to, a region of an RNA that is known to bind to Polycomb repressive complex 1 (PRC1) comprising a motif as described herein, wherein the sequence of the region is selected from the group consisting of SEQ ID NOs:1 to 5893 (human), 5894 to 17415 (human), and 17416 to 36368 (mouse), for use in the treatment of disease, wherein the treatment involves modulating expression of a gene targeted by the RNA, wherein the inhibitory nucleic acid is between 5 and 40 bases in length, and wherein the inhibitory nucleic acid is formulated as a sterile composition.

Further described herein are processs for preparing an inhibitory nucleic acid that specifically binds, or is complementary to, a sequence comprising a motif as described herein within an RNA that is known to bind to Polycomb repressive complex 1 (PRC1), selected from the group consisting of SEQ ID NOs:1 to 5893 (human), 5894 to 17415 (human), and 17416 to 36368 (mouse); the processes include the step of designing and/or synthesizing an inhibitory nucleic acid of between 5 and 40 bases in length, optionally single stranded, that specifically binds to a region of the RNA that binds PRC1.

In some embodiments, the sequence of the designed and/or synthesized inhibitory nucleic acid is a nucleic acid sequence that is complementary to said RNA sequence that binds to PRC1, or is complementary to a portion thereof, said portion having a length of from 5 to 40 contiguous base pairs.

In some embodiments, the inhibitory nucleic acid is for use in the manufacture of a pharmaceutical composition or medicament for use in the treatment of disease, optionally wherein the treatment involves modulating expression of a gene targeted by the RNA binds to PRC1.

In some embodiments, the modulation is increasing expression of a gene and the region of the RNA that binds PRC1 can be in intergenic space mapping to a noncoding RNA, antisense to the coding gene, or in the promoter, 3′UTR, 5′UTR, exons, and introns of a coding gene.

In some embodiments, the modulation is decreasing expression of a gene and the region of the RNA that binds PRC1 can be in intergenic space mapping to a noncoding RNA, antisense to the coding gene, or in the promoter, 3′UTR, 5′UTR, exons, and introns of a coding gene.

In some embodiments,the modulation is to influence gene expression by altering splicing of a gene and the region of the RNA that binds PRC1 can be in intergenic space mapping to a noncoding RNA, antisense to the coding gene, or in the promoter, 3′UTR, 5′UTR, exons, and introns of a coding gene.

Also provided herein are sterile compositions comprising an inhibitory nucleic acid that specifically binds, or is complementary to, a sequence comprising a motif as described herein within an RNA sequence of any one of SEQ ID NOs:1 to 5893 (human), 5894 to 17415 (human), and 17416 to 36368 (mouse) and is capable of modulating expression of a gene targeted by the RNA as set forth in Tables 1-3 of WO 2016/149455. In some embodiments, the composition is for parenteral administration. In some embodiments, the RNA sequence is in the 3′UTR of a gene, and the inhibitory nucleic acid is capable of upregulating or downregulating expression of a gene targeted by the RNA.

Also provided herein is an inhibitory nucleic acid for use in the treatment of disease, wherein said inhibitory nucleic acid specifically binds, or is complementary to, a sequence comprising a motif as described herein within an RNA sequence of any one of SEQ ID NOs:1 to 5893 (human) or 5894 to 17415 (human), and wherein the treatment involves modulating expression of a gene targeted by the RNA according to Tables 1-3 of WO 2016/149455.

The present disclosure also provides methods for modulating gene expression in a cell or a mammal comprising administering to the cell or the mammal an inhibitory nucleic acid that specifically binds, or is complementary to, a sequence comprising a motif as described herein within an RNA sequence of any one of SEQ ID NOs:1 to 5893 (human) or 5894 to 17415 (human) or 17416 to 36368 (mouse), in an amount effective for modulating expression of a gene targeted by the RNA according to Tables 1-3 of WO 2016/149455.

In addition, provided herein are inhibitory nucleic acids of about 5 to 50 bases in length that specifically bind, or are complementary to, at least 5, 6, 7, 8, 9 or 10 consecutive bases within a sequence comprising a motif as described herein within any of SEQ ID NOs:1 to 5893 (human) or 5894 to 17415 (human) or 17416 to 36368 (mouse), optionally for use in the treatment of disease, wherein the treatment involves modulating expression of a gene targeted by the RNA.

In addition, provided are methods for modulating expression of a gene comprising administering to a mammal an inhibitory nucleic acid as described herein in an amount effective for modulating expression of a gene targeted by the RNA as set forth in Tables 1-3 of WO 2016/149455.

In some embodiments, the modulation is upregulating gene expression, optionally wherein the gene targeted by the RNA is selected from the group set forth in Tables 1-3 of WO 2016/149455, and wherein the RNA sequence is listed in the same row as the gene.

In some embodiments, the inhibitory nucleic acid is 5 to 40 bases in length (optionally 12-30, 12-28, or 12-25 bases in length), and optionally the sequence that binds to the motif is centered in the nucleic acid.

In some embodiments, the inhibitory nucleic acid is 10 to 50 bases in length.

In some embodiments, the inhibitory nucleic acid comprises a base sequence at least 90% complementary to at least 10 bases of the RNA sequence.

In some embodiments, the inhibitory nucleic acid comprises a sequence of bases at least 80% or 90% complementary to, e.g., at least 5-30, 10-30, 15-30, 20-30, 25-30 or 5-40, 10-40, 15-40, 20-40, 25-40, or 30-40 bases of the RNA sequence.

In some embodiments, the inhibitory nucleic acid comprises a sequence of bases with up to 3 mismatches (e.g., up to 1, or up to 2 mismatches) in complementary base pairing over 10, 15, 20, 25 or 30 bases of the RNA sequence. In some embodiments, the mismatches are not in the motif-binding region

In some embodiments, the inhibitory nucleic acid comprises a sequence of bases at least 80% complementary to at least 10 bases of the RNA sequence.

In some embodiments,the inhibitory nucleic acid comprises a sequence of bases with up to 3 mismatches over 15 bases of the RNA sequence.

In some embodiments, the inhibitory nucleic acid is single stranded.

In some embodiments, the inhibitory nucleic acid is double stranded.

In some embodiments, the inhibitory nucleic acid comprises one or more modifications comprising: a modified sugar moiety, a modified internucleoside linkage, a modified nucleotide and/or combinations thereof.

In some embodiments, the inhibitory nucleic acid is an antisense oligonucleotide, LNA molecule, PNA molecule, ribozyme or siRNA.

In some embodiments, the inhibitory nucleic acid is double stranded and comprises an overhang (optionally 2-6 bases in length) at one or both termini.

In some embodiments, the inhibitory nucleic acid is selected from the group consisting of antisense oligonucleotides, ribozymes, external guide sequence (EGS) oligonucleotides, siRNA compounds, micro RNAs (miRNAs); small, temporal RNAs (stRNA), and single- or double-stranded RNA interference (RNAi) compounds.

In some embodiments, the RNAi compound is selected from the group consisting of short interfering RNA (siRNA); or a short, hairpin RNA (shRNA); small RNA-induced gene activation (RNAa); and small activating RNAs (saRNAs).

In some embodiments, the antisense oligonucleotide is selected from the group consisting of antisense RNAs, antisense DNAs, and chimeric antisense oligonucleotides.

In some embodiments, the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof.

In some embodiments, the modified sugar moiety comprises a 2′-O-methoxyethyl modified sugar moiety, a 2′-methoxy modified sugar moiety, a 2′-O-alkyl modified sugar moiety, or a bicyclic sugar moiety. In some embodiments, the inhibitory nucleic acids include 2′-OMe, 2′-F, LNA, PNA, FANA, ENA or morpholino modifications.

Further provided are sterile compositions comprising an isolated nucleic acid as described herein.

Further, provided herein are methods of inducing expression of a target gene in a cell, the method comprising delivering to the cell a single stranded oligonucleotide of 5 to 40 nucleotides in length having a region of complementarity that is complementary with at least 5, 6, 7, 8, 9, or 10 contiguous nucleotides including a motif as described herein within a PRC1-binding RNA that inhibits expression of the target gene, wherein the oligonucleotide is complementary to and binds specifically to the PRC1-binding RNA, and wherein the PRC1-binding RNA is transcribed from a sequence of the chromosomal locus of the target gene.

In some embodiments, the RNA is a non-codingRNA.

In some embodiments, the methods include detecting expression of the PRC1-binding RNA in the cell, wherein expression of the PRC1-binding RNA in the cell indicates that the single stranded oligonucleotide is suitable for increasing expression of the target gene in the cell.

In some embodiments, the methods include detecting a change in expression of the target gene following delivery of the single stranded oligonucleotide to the cell, wherein an increase in expression of the target gene compared with an appropriate control cell indicates effectiveness of the single stranded oligonucleotide.

In some embodiments, the methods include detecting a change in recruitment of PRC1 to the target gene in the cell following delivery of the single stranded oligonucleotide to the cell, wherein a decrease in recruitment compared with an appropriate control cell indicates effectiveness of the single stranded oligonucleotide.

In some embodiments, the cell is in vitro.

In some embodiments, the cell is in vivo.

In some embodiments, at least one nucleotide of the oligonucleotide is a modified nucleotide.

In some embodiments, the PRC1-binding RNA is transcribed from the same strand as the target gene in a genomic region containing the target gene.

In some embodiments, the oligonucleotide has complementarity to a region of the PRC1-binding RNA comprising a motif as described herein and transcribed from a portion of the target gene corresponding to an exon.

In some embodiments, the oligonucleotide has complementarity to a region of the PRC1-binding RNA comprising a motif as described herein and transcribed from the same strand as the target gene within a chromosomal region within −2.0 kb to +0.001 kb of the transcription start site of the target gene.

In some embodiments, the oligonucleotide has complementarity to a region of the PRC1-binding RNA comprising a motif as described herein and transcribed from the opposite strand of the target gene within a chromosomal region within −0.5 to +0.1 kb of the transcription start site of the target gene.

In some embodiments, the oligonucleotide has complementarity to the PRC1-binding RNA in a region of the PRC1-binding RNA comprising a motif as described herein, that optionally forms a stem-loop structure.

In some embodiments, at least one nucleotide of the oligonucleotide is an RNA or DNA nucleotide.

In some embodiments, at least one nucleotide of the oligonucleotide is a ribonucleic acid analogue comprising a ribose ring having a bridge between its 2′-oxygen and 4′-carbon.

In some embodiments, the ribonucleic acid analogue comprises a methylene bridge between the 2′-oxygen and the 4′-carbon.

In some embodiments, at least one nucleotide of the oligonucleotide comprises a modified sugar moiety.

In some embodiments, the modified sugar moiety comprises a 2′-O-methoxyethyl modified sugar moiety, a 2′-methoxy modified sugar moiety, a 2′-O-alkyl modified sugar moiety, or a bicyclic sugar moiety.

In some embodiments, the oligonucleotide comprises at least one modified internucleoside linkage.

In some embodiments, the at least one modified internucleoside linkage is selected from phosphorothioate, phosphorodithioate, alkylphosphonothioate, phosphoramidate, carbamate, carbonate, phosphate triester, acetamidate, carboxymethyl ester, and combinations thereof.

In some embodiments, the oligonucleotide is configured such that hybridization of the single stranded oligonucleotide to the PRC1-binding RNA does not activate an RNAse H pathway in the cell.

In some embodiments, the oligonucleotide is configured such that hybridization of the single stranded oligonucleotide to the PRC1-binding RNA does not induce substantial cleavage or degradation of the PRC1-binding RNA in the cell.

In some embodiments, the oligonucleotide is configured such that hybridization of the single stranded oligonucleotide to the PRC1-binding RNA interferes with interaction of the RNA with PRC1 in the cell.

In some embodiments, the target gene is a protein-coding gene.

In some embodiments, the chromosomal locus of the target gene is an endogenous gene of an autosomal chromosome.

In some embodiments, the cell is a cell of a male subject.

In some embodiments, the oligonucleotide has complementarity to a region of the PRC1-binding RNA transcribed from a portion of the target gene corresponding to an intron-exon junction or an intron.

In some embodiments, the oligonucleotide has complementarity to a region of the PRC1-binding RNA transcribed from a portion of the target gene corresponding to a translation initiation region or a translation termination region.

In some embodiments, the oligonucleotide has complementarity to a region of the PRC1-binding RNA transcribed from a portion of the target gene corresponding to a promoter.

In some embodiments, the oligonucleotide has complementarity to a region of the PRC1-binding RNA transcribed from a portion of the target gene corresponding to a 5′-UTR.

In some embodiments, the oligonucleotide has complementarity to a region of the PRC1-binding RNA transcribed from a portion of the target gene corresponding to a 3′-UTR.

In some or any embodiments, the inhibitory nucleic acid is an oligomeric base compound or oligonucleotide mimetic that hybridizes to at least a portion of the target nucleic acid and modulates its function. In some or any embodiments, the inhibitory nucleic acid is single stranded or double stranded. A variety of exemplary inhibitory nucleic acids are known and described in the art. In some examples, the inhibitory nucleic acid is an antisense oligonucleotide, locked nucleic acid (LNA) molecule, peptide nucleic acid (PNA) molecule, ribozyme, siRNA, antagomirs, external guide sequence (EGS) oligonucleotide, microRNA (miRNA), small, temporal RNA (stRNA), or single- or double-stranded RNA interference (RNAi) compounds. It is understood that the term “LNA molecule” refers to a molecule that comprises at least one LNA modification; thus LNA molecules may have one or more locked nucleotides (conformationally constrained) and one or more non-locked nucleotides. It is also understood that the term “LNA” includes a nucleotide that comprises any constrained sugar that retains the desired properties of high affinity binding to complementary RNA, nuclease resistance, lack of immune stimulation, and rapid kinetics. Exemplary constrained sugars include those listed below. Similarly, it is understood that the term “PNA molecule” refers to a molecule that comprises at least one PNA modification and that such molecules may include unmodified nucleotides or internucleoside linkages.

In some or any embodiments, the inhibitory nucleic acid comprises at least one nucleotide and/or nucleoside modification (e.g., modified bases or with modified sugar moieties), modified internucleoside linkages, and/or combinations thereof. Thus, inhibitory nucleic acids can comprise natural as well as modified nucleosides and linkages. Examples of such chimeric inhibitory nucleic acids, including hybrids or gapmers, are described below.

In some embodiments, the inhibitory nucleic acid comprises one or more modifications comprising: a modified sugar moiety, and/or a modified internucleoside linkage, and/or a modified nucleotide and/or combinations thereof. In some embodiments, the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof. In some embodiments, the modified sugar moiety comprises a 2′-O-methoxyethyl modified sugar moiety, a 2′-methoxy modified sugar moiety, a 2′-O-alkyl modified sugar moiety, or a bicyclic sugar moiety. Other examples of modifications include locked nucleic acid (LNA), peptide nucleic acid (PNA), arabinonucleic acid (ANA), optionally with 2′-F modification, 2′-fluoro-D-Arabinonucleic acid (FANA), phosphorodiamidate morpholino oligomer (PMO), ethylene-bridged nucleic acid (ENA), optionally with 2′-O,4′-C-ethylene bridge, and bicyclic nucleic acid (BNA). Yet other examples are described below and/or are known in the art.

In some embodiments, the inhibitory nucleic acid is 5-40 bases in length (e.g., 12-30, 12-28, 12-25). The inhibitory nucleic acid may also be 10-50, or 5-50 bases length. For example, the inhibitory nucleic acid may be one of any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases in length. In some embodiments, the inhibitory nucleic acid is double stranded and comprises an overhang (optionally 2-6 bases in length) at one or both termini. In other embodiments, the inhibitory nucleic acid is double stranded and blunt-ended. In some embodiments, the inhibitory nucleic acid comprises or consists of a sequence of bases at least 80% or 90% complementary to, e.g., at least 5, 10, 15, 20, 25 or 30 bases of, or up to 30 or 40 bases of, the target RNA, or comprises a sequence of bases with up to 3 mismatches (e.g., up to 1, or up to 2 mismatches) over 10, 15, 20, 25 or 30 bases of the target RNA.

Thus, the inhibitory nucleic acid can comprise or consist of a sequence of bases at least 80% complementary to at least 10 contiguous bases of the target RNA comprising a motif as described herein, or at least 80% complementary to at least 15, or 15-30, or 15-40 contiguous bases of the target RNA comprising a motif as described herein, or at least 80% complementary to at least 20, or 20-30, or 20-40 contiguous bases of the target RNA comprising a motif as described herein, or at least 80% complementary to at least 25, or 25-30, or 25-40 contiguous bases of the target RNA comprising a motif as described herein, or at least 80% complementary to at least 30, or 30-40 contiguous bases of the target RNA comprising a motif as described herein, or at least 80% complementary to at least 40 contiguous bases of the target RNA comprising a motif as described herein. Moreover, the inhibitory nucleic acid can comprise or consist of a sequence of bases at least 90% complementary to at least 10 contiguous bases of the target RNA comprising a motif as described herein, or at least 90%complementary to at least 15, or 15-30, or 15-40 contiguous bases of the target RNA comprising a motif as described herein, or at least 90% complementary to at least 20, or 20-30, or 20-40 contiguous bases of the target RNA comprising a motif as described herein, or at least 90% complementary to at least 25, or 25-30, or 25-40 contiguous bases of the target RNA comprising a motif as described herein, or at least 90% complementary to at least 30, or 30-40 contiguous bases of the target RNA comprising a motif as described herein, or at least 90% complementary to at least 40 contiguous bases of the target RNA comprising a motif as described herein. Similarly, the inhibitory nucleic acid can comprise or consist of a sequence of bases fully complementary to at least 5, 10, or 15 contiguous bases of the target RNA comprising a motif as described herein.

In some or any embodiments, the inhibitory nucleic acid is 5 to 40, or 8 to 40, or 10 to 50 bases in length (e.g., 12-30, 12-28, 12-25, 5-25, or 10-25, bases in length), and comprises a sequence of bases with up to 3 mismatches in complementary base pairing over 15 bases of , or up to 2 mismatches over 10 bases.

In an additional aspect, the invention provides methods for enhancing pluripotency of a stem cell. The methods include contacting the cell with an inhibitory nucleic acid that specifically binds, or is complementary, to a nucleic acid sequence that is at least about 90%, 91%, 92%, 93%, 94%, 95%, 96%, 97%, 98% or 99% homologous to a sequence comprising a motif as described herein within a PRC1-binding RNA, as referred to in Tables 1-3 of WO 2016/149455. PRC1-binding fragments of murine or orthologous RNAs, including human RNAs, are contemplated in the aforementioned method.

In a further aspect, the invention features methods for enhancing differentiation of a stem cell, the method comprising contacting the cell with an inhibitory nucleic acid that specifically binds, or is complementary, to a PRC1-binding RNA sequence as set forth in SEQ ID NOS. 17416 to 36368 [mouse Peaks] or 1 to 5893 [human Peaks] or 5894 to 17416 [human Peaks identified by LiftOver].

In some embodiments, the stem cell is an embryonic stem cell. In some embodiments, the stem cell is an iPS cell or an adult stem cell.

In an additional aspect, the invention provides sterile compositions including an inhibitory nucleic acid as described herein. In some embodiments, the inhibitory nucleic acid is selected from the group consisting of antisense oligonucleotides, ribozymes, external guide sequence (EGS) oligonucleotides, siRNA compounds, micro RNAs (miRNAs); small, temporal RNAs (stRNA), and single- or double-stranded RNA interference (RNAi) compounds. In some embodiments, the RNAi compound is selected from the group consisting of short interfering RNA (siRNA); or a short, hairpin RNA (shRNA); small RNA-induced gene activation (RNAa); and small activating RNAs (saRNAs).

In some embodiments, the antisense oligonucleotide is selected from the group consisting of antisense RNAs, antisense DNAs, chimeric antisense oligonucleotides, and antisense oligonucleotides.

In some embodiments, the inhibitory nucleic acid comprises one or more modifications comprising: a modified sugar moiety, a modified internucleoside linkage, a modified nucleotide and/or combinations thereof. In some embodiments, the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof. In some embodiments, the modified sugar moiety comprises a 2′-O-methoxyethyl modified sugar moiety, a 2′-methoxy modified sugar moiety, a 2′-O-alkyl modified sugar moiety, or a bicyclic sugar moiety. Other examples of modifications include locked nucleic acid (LNA), peptide nucleic acid (PNA), arabinonucleic acid (ANA), optionally with 2′-F modification, 2′-fluoro-D-Arabinonucleic acid (FANA), phosphorodiamidate morpholino oligomer (PMO), ethylene-bridged nucleic acid (ENA), optionally with 2′-O,4′-C-ethylene bridge, and bicyclic nucleic acid (BNA). Yet other examples are described below and/or are known in the art.

Inhibitory nucleic acids that specifically bind to a sequence comprising a motif as described herein within any of the RNA peaks set forth in any one of SEQ ID NOs: 1 to 5893, 5894 to 17415, or 17416 to 36368, are also contemplated. In particular, the invention features uses of these inhibitory nucleic acids to upregulate expression of any of the genes set forth in Tables 1-3 of WO 2016/149455, for use in treating a disease, disorder, condition or association known in the art (whether in the “opposite strand” column or the “same strand”); upregulations of a set of genes grouped together in any one of the categories is contemplated. In some embodiments it is contemplated that expression may be increased by at least about 15-fold, 20-fold, 30-fold, 40-fold, 50-fold or 100-fold, or any range between any of the foregoing numbers. In other experiments, increased mRNA expression has been shown to correlate to increased protein expression.

Thus, in various aspects, the invention features inhibitory nucleic acids that specifically bind to motifs as described herien within any of the RNA sequences as set forth in SEQ ID NOs:1 to 5893 (human), 5894 to 17415 (human), and 17416 to 36368 (mouse) or of any of Tables 1-3 of WO 2016/149455, for use in modulating expression of a group of reference genes that fall within any one or more of the categories set forth in the tables, and for treating corresponding diseases, disorders or conditions.

In another aspect, the invention also features inhibitory nucleic acids that specifically bind, or are complementary, to motifs as described herien within any of the RNA sequences of SEQ ID NOS: 17416 to 36368 [mouse Peaks] or 1 to 5893 [human Peaks] or 5894 to 17416 [human Peaks identified by LiftOver], whether in the “opposite strand” column or the “same strand” column of Tables 1-3 of WO 2016/149455. In some embodiments, the inhibitory nucleic acid is provided for use in a method of modulating expression of a gene targeted by the PRC1-binding RNA (e.g., an intersecting or nearby gene, as set forth in any of Tables 1-4 of WO 2016/149455). Such methods may be carried out in vitro, ex vivo, or in vivo. In some embodiments, the inhibitory nucleic acid is provided for use in methods of treating disease, e.g. as described below. The treatments may involve modulating expression (either up or down) of a gene targeted by the PRC1-binding RNA, preferably upregulating gene expression. In some embodiments, the inhibitory nucleic acid is formulated as a sterile composition for parenteral administration. Thus, in one aspect the invention describes a group of inhibitory nucleic acids that specifically bind, or are complementary to, sequences comprising motifs as described herien within a group of RNA sequences, i.e., Peaks, in any one of Tables 1, 2, or 3 of WO 2016/149455. In particular, the invention features uses of such inhibitory nucleic acids to upregulate expression of any of the reference genes set forth in SEQ ID NOs:1 to 5893 (human), 5894 to 17415 (human), and 17416 to 36368 (mouse) or in Tables 1-3 of WO 2016/149455, for use in treating a disease, disorder, or condition.

It is understood that inhibitory nucleic acids of the invention may be complementary to, or specifically bind to, motifs within Peaks, or regions adjacent to (within 100, 200, 300, 400, or 500 nts of) Peaks, as shown in SEQ ID NOs:1 to 5893 (human), 5894 to 17415 (human), and 17416 to 36368 (mouse) or in Tables 1-3 of WO 2016/149455.

Also provided herein are methods for treating a subject with MECP2 Duplication Syndrome. The methods include administering a therapeutically effective amount of an inhibitory nucleic acid targeting a motif within a PRC1-binding region on Mecp2 RNA, e.g., an inhibitory nucleic acid targeting a motif within a sequence within the 3′UTR of Mecp2. In some embodiments, the inhibitory nucleic acid comprises a sequence shown herein, and/or does not comprise any of SEQ ID NOs:36399 to 36404.

Further provided herein are methods for treating a subject with systemic lupus erythematosus. The methods include administering a therapeutically effective amount of an inhibitory nucleic acid targeting a motif within a PRC1-binding region on IRAK1 RNA, e.g., an inhibitory nucleci acid targeting a sequence a motif within within the 3′UTR of IRAK1. In some embodiments, the inhibitory nucleic acid comprises a sequence shown herein, and/or does not comprise any of SEQ ID NOs:36396 to 36398.

In some embodiments, the inhibitory nucleic acid comprises at least one locked nucleotide.

Also provided herein are inhibitory nucleic acids targeting a motif within a PRC1-binding region on Mecp2 RNA, preferably wherein the PRC1 binding region comprises SEQ ID NO:5876 or 5877, and/or preferably an inhibitory nucleic acid targeting a sequence comprising a motif within the 3′UTR of Mecp2, for use in treating a subject with MECP2 Duplication Syndrome. In some embodiments, the inhibitory nucleic acid comprises a sequence shown herein, and/or does not comprise any of SEQ ID NOs:36399 to 36404.

In addition, provided herein are inhibitory nucleic acids targeting a motif within a PRC1-binding region on IRAK1 RNA, preferably wherein the PRC1 binding region comprises SEQ ID NO:5874 or 5875, and/or preferably an inhibitory nucleic acid targeting a sequence comprising a motif within the 3′UTR of IRAK1, for use in treating a subject with systemic lupus erythematosus. In some embodiments, the inhibitory nucleic acid comprises a sequence shown herein, and/or does not comprise any of SEQ ID NOs:36396 to 36398.

In some or any embodiments, the inhibitory nucleic acids are, e.g., about 5 to 40, about 8 to 40, or 10 to 50 bases, or 5 to 50 bases in length. In some embodiments, the inhibitory nucleic acid comprises or consists of a sequence of bases at least 80% or 90% complementary to, e.g., at least 5, 10, 15, 20, 25 or 30 bases of, or up to 30 or 40 bases of, the target RNA (e.g., any one of SEQ ID NOs: 1 to 36,368), or comprises a sequence of bases with up to 3 mismatches (e.g., up to 1, or up to 2 mismatches) over 10, 15, 20, 25 or 30 bases of the target RNA comprising a motif as described herein.

Thus, as noted above, the inhibitory nucleic acid can comprise or consist of a sequence of bases at least 80% complementary to at least 10, or 10-30 or 10-40 contiguous bases of the target RNA comprising a motif as described herein, or at least 80% complementary to at least 15, or 15-30, or 15-40 contiguous bases of the target RNA comprising a motif as described herein, or at least 80% complementary to at least 20, or 20-30, or 20-40 contiguous bases of the target RNA comprising a motif as described herein, or at least 80% complementary to at least 25, or 25-30, or 25-40 contiguous bases of the target RNA comprising a motif as described herein, or at least 80% complementary to at least 30, or 30-40 contiguous bases of the target RNA comprising a motif as described herein, or at least 80% complementary to at least 40 contiguous bases of the target RNA comprising a motif as described herein. Moreover, the inhibitory nucleic acid can comprise or consist of a sequence of bases at least 90% complementary to at least 5, or 5-30 or 5-40 or 8-40 contiguous bases of the target RNA comprising a motif as described herein, or at least 90% complementary to at least 10, or 10-30, or 10-40 contiguous bases of the target RNA comprising a motif as described herein, or at least 90%complementary to at least 15, or 15-30, or 15-40 contiguous bases of the target RNA comprising a motif as described herein, or at least 90% complementary to at least 20, or 20-30, or 20-40 contiguous bases of the target

RNA comprising a motif as described herein, or at least 90% complementary to at least 25, or 25-30, or 25-40 contiguous bases of the target RNA comprising a motif as described herein, or at least 90% complementary to at least 30, or 30-40 contiguous bases of the target RNA comprising a motif as described herein, or at least 90% complementary to at least 40 contiguous bases of the target RNA comprising a motif as described herein. Similarly, the inhibitory nucleic acid can comprise or consist of a sequence of bases fully complementary to at least 5, 10, or 15 contiguous bases of the target RNA comprising a motif as described herein. It is understood that some additional non-complementary bases may be included. It is understood that inhibitory nucleic acids that comprise such sequences of bases as described may also comprise other non-complementary bases. For example, an inhibitory nucleic acid can be 20 bases in total length but comprise a 15 base portion that is fully complementary to 15 bases of the target RNA comprising a motif as described herein. Similarly, an inhibitory nucleic acid can be 20 bases in total length but comprise a 15 base portion that is at least 80% complementary to 15 bases of the target RNA comprising a motif as described herein. Preferably the portion that is complementary to the motif sequence is 100% complementary.

Complementarity can also be referenced in terms of the number of mismatches in complementary base pairing, as noted above. Thus, the inhibitory nucleic acid can comprise or consist of a sequence of bases with up to 3 mismatches over 10 contiguous bases of the target RNA, or up to 3 mismatches over 15 contiguous bases of the target RNA, or up to 3 mismatches over 20 contiguous bases of the target RNA, or up to 3 mismatches over 25 contiguous bases of the target RNA, or up to 3 mismatches over 30 contiguous bases of the target RNA. Similarly, the inhibitory nucleic acid can comprise or consist of a sequence of bases with up to 2 mismatches over 10 contiguous bases of the target RNA, or up to 2 mismatches over 15 contiguous bases of the target RNA, or up to 2 mismatches over 20 contiguous bases of the target RNA, or up to 2 mismatches over 25 contiguous bases of the target RNA, or up to 2 mismatches over 30 contiguous bases of the target RNA. Similarly, the the inhibitory nucleic acid can comprise or consist of a sequence of bases with one mismatch over 10, 15, 20, 25 or 30 contiguous bases of the target RNA.

In some or any of the embodiments of inhibitory nucleic acids described herein (e.g. in the summary, detailed description, or examples of embodiments) or the processes for designing or synthesizing them, the inhibitory nucleic acids may optionally exclude (a) any LNA that disrupts binding of PRC2 to an RNA, e.g., as describe in WO 2012/087983 or WO 2012/065143; (b) any one or more of the specific inhibitory nucleic acids made or actually disclosed (i.e. specific chemistry, single or double-stranded, specific modifications, and specific base sequence), set forth in WO 2012/065143 or WO 2012/087983; and/or the general base sequence of any one or more of the inhibitory nucleic acids of (b); and/or (c) the group of inhibitory nucleic acids that specifically bind or are complementary to the same specific portion of RNA (a stretch of contiguous bases) as any one or more of the inhibitory nucleic acids of (a); as disclosed in any one or more of the following publications: as targeting ANRIL RNA (as described in Yap et al., Mol Cell. 2010 Jun. 11; 38(5):662-74) HOTAIR RNA (Rinn et al., 2007), Tsix, RepA, or Xist RNAs ((Zhao et al., 2008) [SEQ ID NOs: 936166-936170 of WO 2012/087983], or (Sarma et al., 2010) [SEQ ID NOs: 936177-936186 of WO 2012/087983] or (Zhao et al., 2010) [SEQ ID NOs: 936187-936188 of WO 2012/087983] or (Prasnath et al., 2005) [SEQ ID NOs: 936173-936176 of WO 2012/087983] or (Shamovsky et al., 2006) [SEQ ID NO: 936172 of WO 2012/087983] or (Mariner et al., 2008) [SEQ ID NO: 936171 of WO 2012/087983] or (Sunwoo et al., 2008) or (Bernard et al., 2010) [SEQ ID NO: 936189 of WO 2012/087983]; or as targeting short RNAs of 50-200 nt that are identified as candidate PRC2 regulators (Kanhere et al., 2010); or (Kuwabara et al., US 2005/0226848) [SEQ ID NOs: 936190-936191 of WO 2012/087983] or (Li et al., US 2010/0210707) [SEQ ID NOs: 936192-936227 of WO 2012/087983] or (Corey et al., U.S. Pat. No. 7,709,456) [SEQ ID NOs: 936228-936245] or (Mattick et al., WO 2009/124341), or (Corey et al., US 2010/0273863) [SEQ ID NOs: 936246-936265 of WO 2012/087983], or (Wahlstedt et al., US 2009/0258925) [SEQ ID NOs: 935060-935126 of WO 2012/087983], or BACE: US 2009/0258925 [SEQ ID NOs: 935060-935126 of WO 2012/087983]; ApoA1: US 2010/0105760/EP235283 [SEQ ID NOs: 935127-935299 of WO 2012/087983], P73, p53, PTEN, WO 2010/065787 A2/EP2370582 [SEQ ID NOs: 935300-935345 of WO 2012/087983]; SIRT1: WO 2010/065662 A2/EP09831068 [SEQ ID NOs: : 935346-935392 of WO 2012/087983]; VEGF: WO 2010/065671 A2/EP2370581 [SEQ ID NOs: 935393-935403 of WO 2012/087983]; EPO: WO 2010/065792 A2/EP09831152 [SEQ ID NOs: 935404-935412 of WO 2012/087983]; BDNF: WO2010/093904 [SEQ ID NOs: 935413-935423 of WO 2012/087983], DLK1: WO 2010/107740 [SEQ ID NOs: 935424-935430 of WO 2012/087983]; NRF2/NFE2L2: WO 2010/107733 [SEQ ID NOs: 935431-935438 of WO 2012/087983]; GDNF: WO 2010/093906 [SEQ ID NOs: 935439-935476 of WO 2012/087983]; SOX2, KLF4, Oct3A/B, “reprogramming factors: WO 2010/135329 [SEQ ID NOs: 935477-935493 of WO 2012/087983]; Dystrophin: WO 2010/129861 [SEQ ID NOs: 935494-935525 of WO 2012/087983]; ABCA1, LCAT, LRP1, ApoE, LDLR, ApoA1: WO 2010/129799 [SEQ ID NOs: 935526-935804 of WO 2012/087983]; HgF: WO 2010/127195 [SEQ ID NOs: 935805-935809 of WO 2012/087983]; TTP/Zfp36: WO 2010/129746[SEQ ID NOs: 935810-935824 of WO 2012/087983]; TFE3, IRS2: WO 2010/135695 [SEQ ID NOs: 935825-935839 of WO 2012/087983]; RIG1, MDA5, IFNA1: WO 2010/138806 [SEQ ID NOs: 935840-935878 of WO 2012/087983]; PON1: WO 2010/148065 [SEQ ID NOs: 935879-935885 of WO 2012/087983]; Collagen: WO/2010/148050 [SEQ ID NOs: 935886-935918 of WO 2012/087983]; Dyrk1A, Dscr1, “Down Syndrome Gene”: WO/2010/151674 [SEQ ID NOs: 935919-935942 of WO 2012/087983]; TNFR2: WO/2010/151671 [SEQ ID NOs: 935943-935951 of WO 2012/087983]; Insulin: WO/2011/017516 [SEQ ID NOs: 935952-935963 of WO 2012/087983]; ADIPOQ: WO/2011/019815 [SEQ ID NOs: 935964-935992 of WO 2012/087983]; CHIP: WO/2011/022606 [SEQ ID NOs: 935993-936004 of WO 2012/087983]; ABCB1: WO/2011/025862 [SEQ ID NOs: 936005-936014 of WO 2012/087983]; NEUROD1, EUROD1, HNF4A, MAFA, PDX, KX6, “Pancreatic development gene”: WO/2011/085066 [SEQ ID NOs: 936015-936054 of WO 2012/087983]; MBTPS1: WO/2011/084455 [SEQ ID NOs: 936055-936059 of WO 2012/087983]; SHBG: WO/2011/085347 [SEQ ID NOs: 936060-936075 of WO 2012/087983]; IRF8: WO/2011/082409 [SEQ ID NOs: 936076-936080 of WO 2012/087983]; UCP2: WO/2011/079263 [SEQ ID NOs: 936081-936093 of WO 2012/087983]; HGF: WO/2011/079261 [SEQ ID NOs: 936094-936104 of WO 2012/087983]; GH: WO/2011/038205 [SEQ ID NOs: 936105-936110 of WO 2012/087983]; IQGAP: WO/2011/031482 [SEQ ID NOs: 936111-936116 of WO 2012/087983]; NRF1: WO/2011/090740 [SEQ ID NOs: 936117-936123 of WO 2012/087983]; P63: WO/2011/090741 [SEQ ID NOs: 936124-936128 of WO 2012/087983]; RNAseH1: WO/2011/091390 [SEQ ID NOs: 936129-936140 of WO 2012/087983]; ALOX12B: WO/2011/097582 [SEQ ID NOs: 936141-936146 of WO 2012/087983]; PYCR1: WO/2011/103528 [SEQ ID NOs: 936147-936151 of WO 2012/087983]; CSF3: WO/2011/123745 [SEQ ID NOs: 936152-936157 of WO 2012/087983]; FGF21: WO/2011/127337 [SEQ ID NOs: 936158-936165 of WO 2012/087983]; SIRTUIN (SIRT): WO2011/139387 [SEQ ID NOs: 936266-936369 and 936408-936425 of WO 2012/087983]; PAR4: WO2011/143640 [SEQ ID NOs: 936370-936376 and 936426 of WO 2012/087983]; LHX2: WO2011/146675 [SEQ ID NOs: 936377-936388 and 936427-936429 of WO 2012/087983]; BCL2L11: WO2011/146674 [SEQ ID NO: 936389-936398 and 936430-936431 of WO 2012/087983]; MSRA: WO2011/150007 [SEQ ID NOs: 936399-936405 and 936432 of WO 2012/087983]; ATOH1: WO2011/150005 [SEQ ID NOs: 936406-936407 and 936433 of WO 2012/087983] of which each of the foregoing is incorporated by reference in its entirety herein. In some or any of the embodiments, optionally excluded from the invention are of inhibitory nucleic acids that specifically bind to, or are complementary to, any one or more of the following regions: Nucleotides 1-932 of SEQ ID NO: 935128 of WO 2012/087983; Nucleotides 1-1675 of SEQ ID NO: 935306 of WO 2012/087983; Nucleotides 1-518 of SEQ ID NO: 935307 of WO 2012/087983; Nucleotides 1-759 of SEQ ID NO: 935308 of WO 2012/087983; Nucleotides 1-25892 of SEQ ID NO: 935309 of WO 2012/087983; Nucleotides 1-279 of SEQ ID NO: 935310 of WO 2012/087983; Nucleotides 1-1982 of SEQ ID NO: 935311 of WO 2012/087983; Nucleotides 1-789 of SEQ ID NO: 935312 of WO 2012/087983; Nucleotides 1-467 of SEQ ID NO: 935313 of WO 2012/087983; Nucleotides 1-1028 of SEQ ID NO: 935347 of WO 2012/087983; Nucleotides 1-429 of SEQ ID NO: 935348 of WO 2012/087983; Nucleotides 1-156 of SEQ ID NO: 935349 of WO 2012/087983; Nucleotides 1-593 of SEQ ID NO:935350 of WO 2012/087983; Nucleotides 1-643 of SEQ ID NO: 935395 of WO 2012/087983; Nucleotides 1-513 of SEQ ID NO: 935396 of WO 2012/087983; Nucleotides 1-156 of SEQ ID NO: 935406 of WO 2012/087983; Nucleotides 1-3175 of SEQ ID NO: 935414 of WO 2012/087983; Nucleotides 1-1347 of SEQ ID NO: 935426 of WO 2012/087983; Nucleotides 1-5808 of SEQ ID NO: 935433 of WO 2012/087983; Nucleotides 1-237 of SEQ ID NO: 935440 of WO 2012/087983; Nucleotides 1-1246 of SEQ ID NO: 935441 of WO 2012/087983; Nucleotides 1-684 of SEQ ID NO: 935442 of WO 2012/087983; Nucleotides 1-400 of SEQ ID NO: 935473 of WO 2012/087983; Nucleotides 1-619 of SEQ ID NO: 935474 of WO 2012/087983;Nucleotides 1-813 of SEQ ID NO: 935475 of WO 2012/087983; Nucleotides 1-993 of SEQ ID NO: 935480 of WO 2012/087983; Nucleotides 1-401 of SEQ ID NO: 935480 of WO 2012/087983; Nucleotides 1-493 of SEQ ID NO: 935481 of WO 2012/087983; Nucleotides 1-418 of SEQ ID NO: 935482 of WO 2012/087983; Nucleotides 1-378 of SEQ ID NO: 935496 of WO 2012/087983; Nucleotides 1-294 of SEQ ID NO: 935497 of WO 2012/087983; Nucleotides 1-686 of SEQ ID NO: 935498 of WO 2012/087983; Nucleotides 1-480 of SEQ ID NO: 935499 of WO 2012/087983; Nucleotides 1-501 of SEQ ID NO: 935500 of WO 2012/087983; Nucleotides 1-1299 of SEQ ID NO: 935533 of WO 2012/087983; Nucleotides 1-918 of SEQ ID NO: 935534 of WO 2012/087983; Nucleotides 1-1550 of SEQ ID NO: 935535 of WO 2012/087983; Nucleotides 1-329 of SEQ ID NO: 935536 of WO 2012/087983; Nucleotides 1-1826 of SEQ ID NO: 935537 of WO 2012/087983; Nucleotides 1-536 of SEQ ID NO: 935538 of WO 2012/087983; Nucleotides 1-551 of SEQ ID NO: 935539 of WO 2012/087983; Nucleotides 1-672 of SEQ ID NO: 935540 of WO 2012/087983; Nucleotides 1-616 of SEQ ID NO: 935541 of WO 2012/087983; Nucleotides 1-471 of SEQ ID NO: 935542 of WO 2012/087983; Nucleotides 1-707 of SEQ ID NO: 935543 of WO 2012/087983; Nucleotides 1-741 of SEQ ID NO: 935544 of WO 2012/087983; Nucleotides 1-346 of SEQ ID NO: 935545 of WO 2012/087983; Nucleotides 1-867 of SEQ ID NO: 935546 of WO 2012/087983; Nucleotides 1-563 of SEQ ID NO: 935547 of WO 2012/087983; Nucleotides 1-970 of SEQ ID NO: 935812 of WO 2012/087983; Nucleotides 1-1117 of SEQ ID NO: 935913 of WO 2012/087983; Nucleotides 1-297 of SEQ ID NO: 935814 of WO 2012/087983; Nucleotides 1-497 of SEQ ID NO: 935827 of WO 2012/087983; Nucleotides 1-1267 of SEQ ID NO: 935843 of WO 2012/087983; Nucleotides 1-586 of SEQ ID NO: 935844 of WO 2012/087983; Nucleotides 1-741 of SEQ ID NO: 935845 of WO 2012/087983; Nucleotides 1-251 of SEQ ID NO: 935846 of WO 2012/087983; Nucleotides 1-681 of SEQ ID NO: 935847 of WO 2012/087983; Nucleotides 1-580 of SEQ ID NO: 935848 of WO 2012/087983; Nucleotides 1-534 of SEQ ID NO: 935880 of WO 2012/087983; Nucleotides 1-387 of SEQ ID NO: 935889 of WO 2012/087983; Nucleotides 1-561 of SEQ ID NO: 935890 of WO 2012/087983; Nucleotides 1-335 of SEQ ID NO: 935891 of WO 2012/087983; Nucleotides 1-613 of SEQ ID NO: 935892 of WO 2012/087983; Nucleotides 1-177 of SEQ ID NO: 935893 of WO 2012/087983; Nucleotides 1-285 of SEQ ID NO: 935894 of WO 2012/087983; Nucleotides 1-3814 of SEQ ID NO: 935921 of WO 2012/087983; Nucleotides 1-633 of SEQ ID NO: 935922 of WO 2012/087983; Nucleotides 1-497 of SEQ ID NO: 935923 Nucleotides 1-545 of SEQ ID NO: 935924 of WO 2012/087983; Nucleotides 1-413 of SEQ ID NO: 935950 of WO 2012/087983; Nucleotides 1-413 of SEQ ID NO: 935951 of WO 2012/087983; Nucleotides 1-334 of SEQ ID NO: 935962 of WO 2012/087983; Nucleotides 1-582 of SEQ ID NO: 935963 of WO 2012/087983; Nucleotides 1-416 of SEQ ID NO: 935964 of WO 2012/087983; Nucleotides 1-3591 of SEQ ID NO: 935990 of WO 2012/087983; Nucleotides 1-875 of SEQ ID NO: 935991 of WO 2012/087983; Nucleotides 1-194 of SEQ ID NO: 935992 of WO 2012/087983; Nucleotides 1-2074 of SEQ ID NO: 936003 of WO 2012/087983; Nucleotides 1-1237 of SEQ ID NO: 936004 of WO 2012/087983; Nucleotides 1-4050 of SEQ ID NO: 936013 of WO 2012/087983; Nucleotides 1-1334 of SEQ ID NO: 936014 of WO 2012/087983; Nucleotides 1-1235 of SEQ ID NO: 936048 of WO 2012/087983; Nucleotides 1-17,964 of SEQ ID NO: 936049 of WO 2012/087983; Nucleotides 1-50,003 of SEQ ID NO: 936050 of WO 2012/087983; Nucleotides 1-486 of SEQ ID NO: 936051 of WO 2012/087983; Nucleotides 1-494 of SEQ ID NO: 936052 of WO 2012/087983; Nucleotides 1-1992 of SEQ ID NO: 936053 of WO 2012/087983; Nucleotides 1-1767 of SEQ ID NO: 936054 of WO 2012/087983; Nucleotides 1-1240 of SEQ ID NO: 936059 of WO 2012/087983; Nucleotides 1-3016 of SEQ ID NO: 936074 of WO 2012/087983; Nucleotides 1-1609 of SEQ ID NO: 936075 of WO 2012/087983; Nucleotides 1-312 of SEQ ID NO: 936080 of WO 2012/087983; Nucleotides 1-243 of SEQ ID NO: 936092 of WO 2012/087983; Nucleotides 1-802 of SEQ ID NO: 936093 of WO 2012/087983; Nucleotides 1-514 of SEQ ID NO: 936102 of WO 2012/087983; Nucleotides 1-936 of SEQ ID NO: 936103 of WO 2012/087983; Nucleotides 1-1075 of SEQ ID NO: 936104 of WO 2012/087983; Nucleotides 1-823 of SEQ ID NO: 936110 of WO 2012/087983; Nucleotides 1-979 of SEQ ID NO: 936116 of WO 2012/087983; Nucleotides 1-979 of SEQ ID NO: 936123 of WO 2012/087983; Nucleotides 1-288 of SEQ ID NO: 936128 of WO 2012/087983; Nucleotides 1-437 of SEQ ID NO: 936137 of WO 2012/087983; Nucleotides 1-278 of SEQ ID NO: 936138 of WO 2012/087983; Nucleotides 1-436 of SEQ ID NO: 936139 of WO 2012/087983; Nucleotides 1-1140 of SEQ ID NO: 936140 of WO 2012/087983; Nucleotides 1-2082 of SEQ ID NO: 936146 of WO 2012/087983; Nucleotides 1-380 of SEQ ID NO: 936151 of WO 2012/087983; Nucleotides 1-742 of SEQ ID NO: 936157 of WO 2012/087983; Nucleotides 1-4246 of SEQ ID NO: 936165 of WO 2012/087983; Nucleotides 1-1028 of SEQ ID NO: 936408 of WO 2012/087983; Nucleotides 1-429 of SEQ ID NO: 936409 of WO 2012/087983; Nucleotides 1-508 of SEQ ID NO: 936410 of WO 2012/087983; Nucleotides 1-593 of SEQ ID NO: 936411 of WO 2012/087983; Nucleotides 1-373 of SEQ ID NO: 936412 of WO 2012/087983; Nucleotides 1-1713 of SEQ ID NO: 936413 of WO 2012/087983; Nucleotides 1-660 of SEQ ID NO:936414 of WO 2012/087983; Nucleotides 1-589 of SEQ ID NO: 936415 of WO 2012/087983; Nucleotides 1-726 of SEQ ID NO: 936416 of WO 2012/087983; Nucletides 1-320 of SEQ ID NO: 936417 of WO 2012/087983; Nucletides 1-616 of SEQ ID NO: 936418 of WO 2012/087983; Nucletides 1-492 of SEQ ID NO: 936419 to of WO 2012/087983; Nucletides 1-428 of SEQ ID NO: 936420 of WO 2012/087983; Nucletides 1-4041 of SEQ ID NO: 936421 of WO 2012/087983; Nucletides 1-705 of SEQ ID NO: 936422 of WO 2012/087983; Nucletides 1-2714 of SEQ ID NO: 936423 of WO 2012/087983; Nucletides 1-1757 of SEQ ID NO: 936424 of WO 2012/087983; Nucletides 1-3647 of SEQ ID NO: 936425 of WO 2012/087983; Nucleotides 1-354 of SEQ ID NO: 936426 of WO 2012/087983; Nucleotides 1-2145 of SEQ ID NO: 936427, Nucleotides 1-606 of SEQ ID NO: 936428 of WO 2012/087983; Nucleotides 1-480 of SEQ ID NO: 936429 of WO 2012/087983; Nucleotides 1-3026 of SEQ ID NO: 936430 of WO 2012/087983; Nucleotides 1-1512 of SEQ ID NO: 936431 of WO 2012/087983; Nucleotides 1-3774 of SEQ ID NO: 936432 of WO 2012/087983; Nucleotides 1-589 of SEQ ID NO: 936433.

In some of the embodiments of inhibitory nucleic acids described herein, or processes for designing or synthesizing them, the inhibitory nucleic acids will upregulate gene expression and may specifically bind or specifically hybridize or be complementary to a sequence comprising a motif as described herien within the PRC1-binding RNA that is transcribed from the same strand as a protein coding reference gene. The inhibitory nucleic acid may bind to a region of the PRC1-binding RNA, that originates within or overlaps an intron, exon, intron-exon junction, 5′ UTR, 3′ UTR, a translation initiation region, or a translation termination region of a protein-coding sense-strand of a reference gene (refGene).

In some or any of the embodiments of inhibitory nucleic acids described herein, or processes for designing or syntheisizing them, the inhibitory nucleic acids will upregulate gene expression and may specifically bind or specifically hybridize or be complementary to a sequence comprising a motif as described herien within a PRC1 binding RNA that transcribed from the opposite strand (the antisense-strand) of a protein-coding reference gene.

The inhibitory nucleic acids described herein may be modified, e.g. comprise a modified sugar moiety, a modified internucleoside linkage, a modified nucleotide and/or combinations thereof. In addition, the inhibitory nucleic acids can exhibit one or more of the following properties: do not induce substantial cleavage or degradation of the target RNA; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; may have improved endosomal exit; do interfere with interaction of ncRNA with PRC1, preferably the Ezh2 subunit but optionally the Suz12, Eed, RbAp46/48 subunits or accessory factors such as Jarid2; do decrease histone H3-lysine27 methylation and/or do upregulate gene expression.

In some or any of the embodiments of inhibitory nucleic acids described herein, or processes for designing or synthesizing them, the inhibitory nucleic acids may optionally exclude those that bind DNA of a promoter region, as described in Kuwabara et al., US 2005/0226848 or Li et al., US 2010/0210707 or Corey et al., U.S. Pat. No. 7,709,456 or Mattick et al., WO 2009/124341, or those that bind DNA of a 3′ UTR region, as described in Corey et al., US 2010/0273863.

Inhibitory nucleic acids that are designed to interact with RNA to modulate gene expression are a distinct subset of base sequences from those that are designed to bind a DNA target (e.g., are complementary to the underlying genomic DNA sequence from which the RNA is transcribed).

This application incorporates by reference the entire disclosures of U.S. provisional No. 61/425,174 filed on Dec. 20, 2010, and 61/512,754 filed on Jul. 28, 2011, and International Patent Appliation Nos. PCT/US2011/060493, filed Nov. 12, 2011, and PCT/US2011/065939, filed on Dec. 19, 2011.

In some embodiments, the motif as described herein is a motif as shown in FIG. 2B, FIG. 7D, and/or Table 1.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-F. Denaturing CLIP of CBX7 in ES cells. See also FIG. 8-13.

(Panel A) Schematic workflow for dCLIP assay.

(Panel B) Representative dCLIP experiment. Left panel, autoradiography of dCLIP experiment. Right panel, Western blot with anti-CBX7 antibody for streptavidin pull-down samples. Lanes which contained input samples have been omitted for clarity. Red arrows, Biotagged-CBX7 signal. 3E and 6F are two clonal cell lines expressing physiological levels of Biotagged-CBX7. 3E and 6F are used as biological replicates for CBX7 dCLIP-seq libraries.

(Panel C) Representative CBX7 dCLIP and ChIP profiles for selected genes. DHS, DNAseI-hypersensitive sites from Vierstra et al (Vierstra et al., 2014). Orange boxes, LNA ASO cocktails. Red stars, primer pairs for ChIP-qPCR. Green hexagons, primer pairs for FAIRE-qPCR.

(Panel D) Strand-specific enriched peaks (called by “PeakRanger”) from three individual CLIP libraries were pooled and overlapped peaks were merged into longer regions in a strand-specific manner. Length distribution frequency of the enriched CLIP peaks, as well as mean, median, and standard deviation were calculated.

(Panel E) Comparison of metagene profiles for CBX7 dCLIP-seq peaks and CBX7 ChIP-seq peaks. TSS, transcriptional start site. TTS, transcriptional termination site.

(Panel F) Correlation between gene expression levels and CLIP signal. Black, expressed RefSeq genes with reproducible dCLIP signal. Green, genes with a highest CLIP signals. Red, expressed genes with no reproducible CLIP signals.

FIGS. 2A-D. Identification and characterization of binding motifs for CBX7.

(Panel A) Bioinformatics pipeline: Schematic workflow of search algorithm for CBX7 biding motifs.

(Panel B) Families of binding motifs identified for CBX7 dCLIP. Groups of motifs arranged into families according to similarity.

(Panel C) Abundance of motif families across different transcript features.

(Panel D) Box plot for FAM-occupancy ratios between number of CBX7 binding sites predicted by motif analysis and confirmed by a dCLIP data to a total number of putative binding sites predicted by motif analysis. Occupancy ratio of 1 indicates that all putative binding sites in a specific gene's genomic feature were validated as CBX7-binding sites based on CLIP-seq analysis. Black line depicts median. CDs, coding DNA sequences (coding exons).

FIGS. 3A-D. Spatial arrangement of binding motifs on target transcripts

(Panel A) “Nearest neighbor” analysis for motif families. Note the strong tendency for FAM1 motifs to congregate next to each other.

(Panel B) Distance distribution between motif pairs. While certain motif pairs such as FAM3-FAM3 and FAM4-FAM4 occurred in a very close proximity, other motif pairs such as FAM4-FAM2 exhibited much broader spectra of inter-motif distances.

(Panel C) Histogram plotting the number of CBX7 footprints (dCLIP fibers) with indicated adjacent FAM motifs in the same footprint. Note a tendency of motifs to congregate.

(Panel D) The FAM occupancy ratio on CBX7 footprints with a single FAM (upper graph) versus those with clustered FAMs (lower graph). Congregation of motifs in the 3′UTR regions is positively correlated with higher occupancy ratios, suggesting the possibility of cooperative binding.

FIGS. 4A-B. Secondary structure probing and relationship of CBX7 motif to known motifs. See also FIG. 14.

(Panel A) CBX7 binding motifs bear a significant similarity to the binding motifs of known RNA binding proteins. hPDI motifs were adopted from Xie et al (Xie et al., 2010). RNAcompete motifs were adopted from Ray et al (Ray et al., 2013).

(Panel B) Effect of RNA secondary structure probed in vivo and in vitro on CBX7 RNA binding. IcSHAPE profiles centered on the genomic sequences predicted as carrying CBX7 binding motifs. IcSHAPE analysis was adopted from Spitale et al (Spitale et al., 2015). Purified RNA molecules were subjected to treatment with icSHAPE reagent in vitro or isolated from cells exposed to icSHAPE reagent in cell culture (in vivo). Extent of RNA folding in particular region is determined by its accessibility to modification by icSHAPE reagent with higher icSHAPE signal representing more open structure. RealFAM—depicting sequences predicted by motif analysis and confirmed as actual CBX7 binding sites by dCLIP. PredictedFAM—depicting sequences predicted by motif analysis but lacking a dCLIP signal. Note that despite a marked similarity of the curves between binding and non-binding FAM sequences, average icSHAPE signal in actual binding sequences is significantly higher than in non-binding FAM sequences, reflecting more open overall structures.

FIGS. 5A-G. Validation of CBX7 interactions with transcripts identified by dCLIP

(Panel A) Validation of CBX7 dCLIP data for selected transcripts by nRIP-qPCR. Average fold-enrichment over IgG control is plotted, with standard devations (error bars). U1 small nuclear RNA, negative control.

(Panel B) RNA-EMSAs with purified CBX7 (5.6 μM) and in vitro transcribed RNAs demonstrate direct RNA-protein interactions. Concentrations of RNA: 26.5 nM for Dcaf12l1, 62 nM for Dusp9, 37.9 nM for Calm2. Green arrows, unbound RNA probes. Red asterisks, bound and shifted RNA-protein complexes. Blue arrows, LNA-shifted probes. Red arrowheads, supershifted complexes after gene-specific LNA addition.

(Panel C) Representative EMSA showing titration of CBX7 protein against fixed concentration (40 nM) of the 3′UTR fragment of Dcaf12l1. Red arrows, unbound probe. Black arrows, shifted CBX7-RNA complexes of different mobilities.

(Panel D) Competition assay: Shift of 40 nM Dcaf12l1 3′UTR probe by 2.8 uM of CBX7 is competed away by excess cold Dcaf12l1. Red arrows, unbound probe. Black arrows, shifted CBX7-RNA complexes of different mobilities.

(Panel E) Binding curves for CBX7-3′UTR interactions for selected transcripts. Kd's and Hill coefficients determined by fitting datapoints to sigmoidal plots by non-linear regression (STAR Methods).

(Panel F) RNA-EMSAs with purified CBX7 (5.6 μM) and 100 nM of in vitro-transcribed wildtype oligos bearing a single FAM motif versus their mutated versions. Green arrows, unbound RNA probes. Red arrows, bound and shifted RNA-protein complexes.

(Panel G) FAM3 competition EMSA using 2.8 uM CBX7 and 100 nM labelled Nuck1-FAM3 RNA probe. Increasing concentrations of Nucks1-FAM3 cold competitor were added, as indicated. Green arrows, unbound RNA probes. Red arrows, bound and shifted RNA-protein complexes.

FIGS. 6A-I. Modulation of CBX7-3′UTR interactions results in gene upregulation. See also FIG. 15

(Panel A) LNA administration resulted in gene upregulation, as shown by RT-qPCR. LNA cocktails are used for each transcript (see FIG. 2 and FIG. 12 for map). Expression values are fold-changes in expression compared to cells treated with scrambled LNA. P values determined by t-test from 3 biological replicates.

(Panel B) ChIP-qPCR for CBX7 localization H2AK119Ub levels after LNA administration as indicated. IgG for control ChIP pulldown. Data presented is average+/−S.D of at least three biological replicates.

(Panel C) FAIRE-qPCR analysis of chromatin compaction in the promoter regions (DNAse-sensitive regions) versus regions corresponding to CBX7 ChIP peaks (DNAse-resistant regions) following LNA treatment. Values were normalized to the β-actin locus (constant). Average+/−S.D of at least three biological replicates shown.

(Panel D) RT-qPCR to determine effect of LNA administration on nascent transcripts (intronic primer pairs) compared to total mRNA (inter-exonic primer pairs) levels. Expression levels are relative to those of cells treated with scrambled LNA. P values determined by t-test from 3 biological replicates.

(Panel E) Dcaf12l1 upregulation after LNA treatment depends on CBX7. Relative Dcaf12l1 expression in Cbx7−/− versus wildtype ES cells. RT-pPCR of nascent (intronic primer pairs) versus processed mRNA (inter-exonic primer pairs) is shown. P values determined by t-test from 3 biological replicates.

(Panel F). Probability density functions for CBX7-bound versus unbound transcripts. Relative FPKM values are determined from RNA-seq of the ES cells in which dCLIP was performed. Note that bound transcripts have a tendency towards higher expression.

(Panel G) Western immunoblot for DCAF12L1 and loading control CTCF protein. Western analysis is quantitative and showed linear response between 2.5-20.0 ul of extract for both proteins. Standard curve for the Western analysis displayed Squared correlation coefficients (R²) of approximately 1.0, suggesting an excellent fit of the curve to observed values.

(Panel H) One example of quantitative Western blot analysis for expression of DCAF12L1 protein following treatment with LNA oligomers. Densitometric analysis was performed and values are normalized to control-LNA-treated samples. Western immunoblots appearing in panels G and H, which were part of images generated by Chemidoc MP Imaging System (as described under STAR Methods) were cropped from their original context and recomposed into separate panels for presentation purposes.

(Panel I) Average of three biological replicates of quantitative Western blot analysis for DCAF12L1 protein. Values are fold-changes in protein signal compared to cells treated with scrambled LNA. P values determined by t-test from 3 biological replicates.

FIGS. 7A-F. Identification and characterization of binding motifs for human CBX7 by dCLIP. See also FIG. 16.

(Panel A) Length distribution frequency of the enriched hCBX7 dCLIP peaks, as well as mean, median, and standard deviation.

(Panel B) Metagene profiles for hCBX7 dCLIP-seq peaks shows enrichment at the 3′ end of mRNAs. TSS, transcriptional start site. TTS, transcriptional termination site.

(Panel C) Representative hCBX7 dCLIP profile. BMI1 analysis was performed on previous GRIP dataset (Ray et al., 2016).

(Panel D) Similarity analysis for families of binding motifs identified for hCBX7 and mCBX7 dCLIP. Groups of motifs arranged according to similarity. Note partial clustering of human and mouse motifs.

(Panel E) Validation of CBX7 dCLIP data for selected transcripts by dCLIP-qPCR. Average fold-enrichment over GFP control is plotted, with standard deviations (error bars). PES1 served as a negative control that did not exhibit significant binding to CBX7. P values were determined by t-test from 3 biological replicates.

(Panel F) hCBX7 motifs bear significant similarity to motifs of known RNA binding proteins. hPDI motifs were adopted from Xie et. al. (Xie et al., 2010). RNAcompete motifs were adopted from Ray et al (Ray et al., 2013).

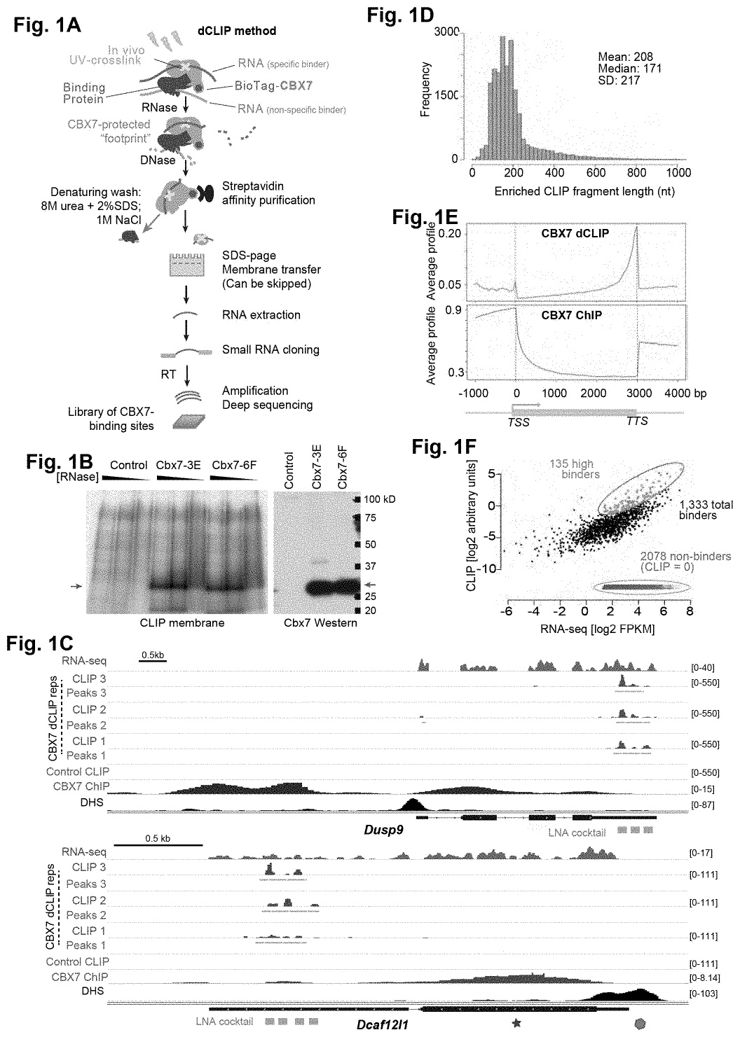

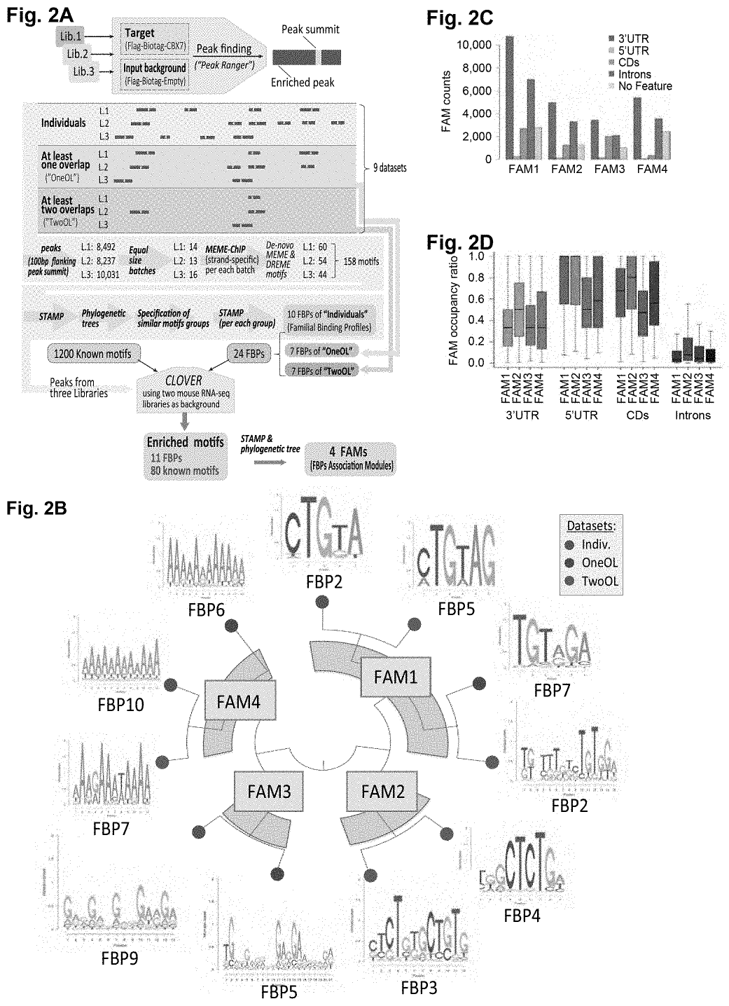

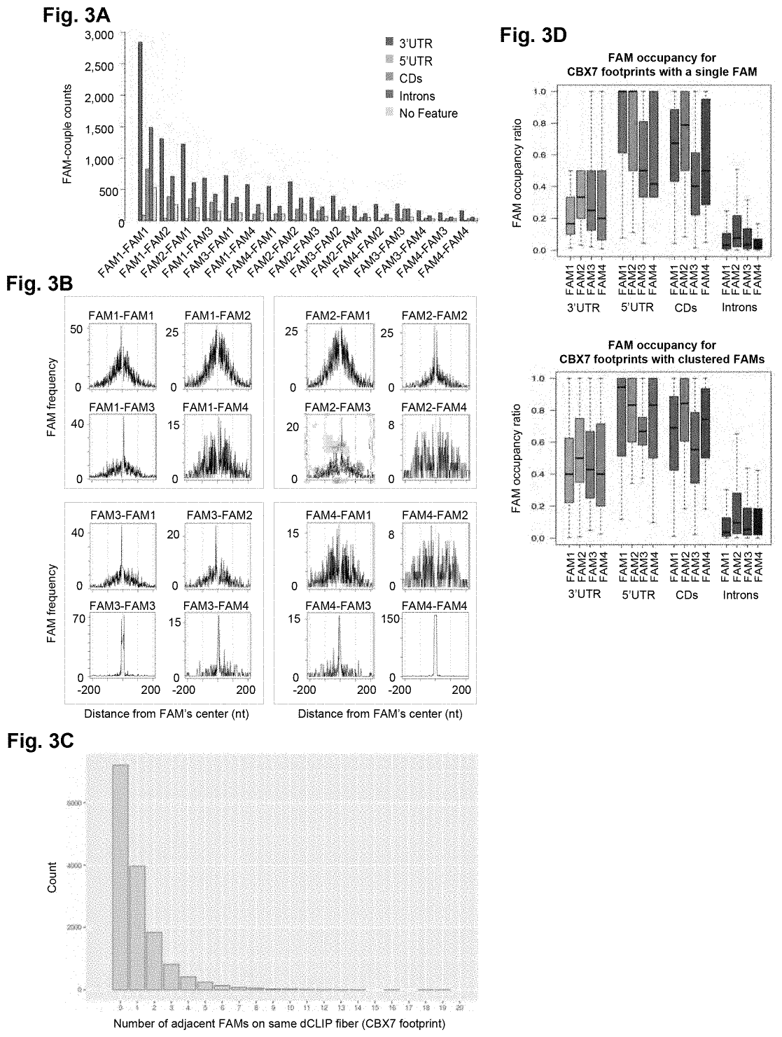

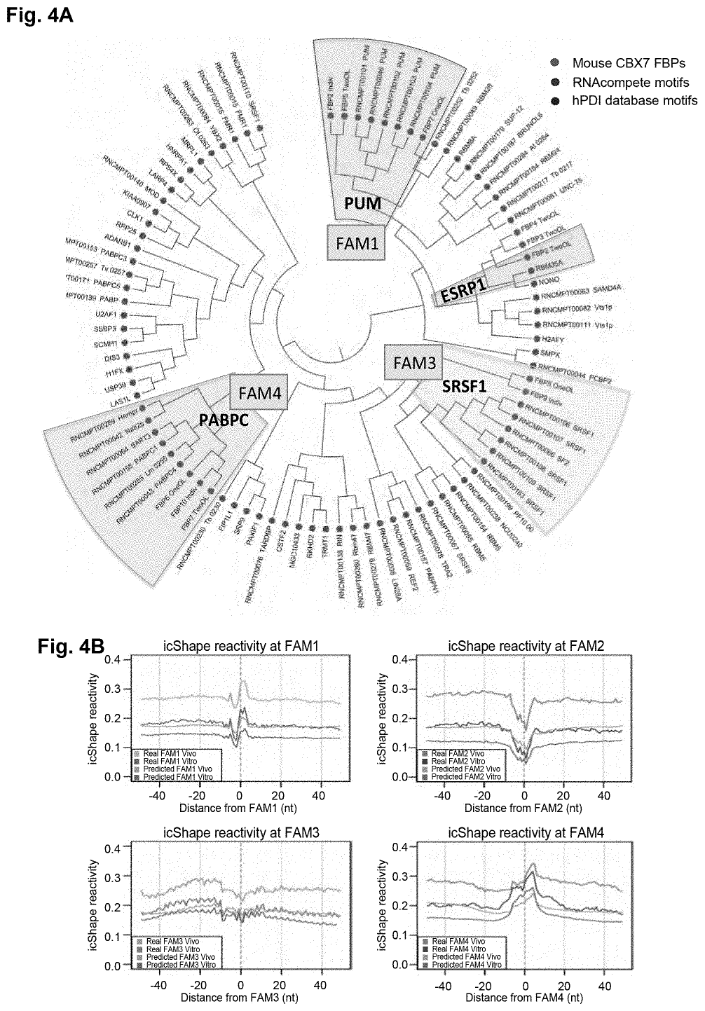

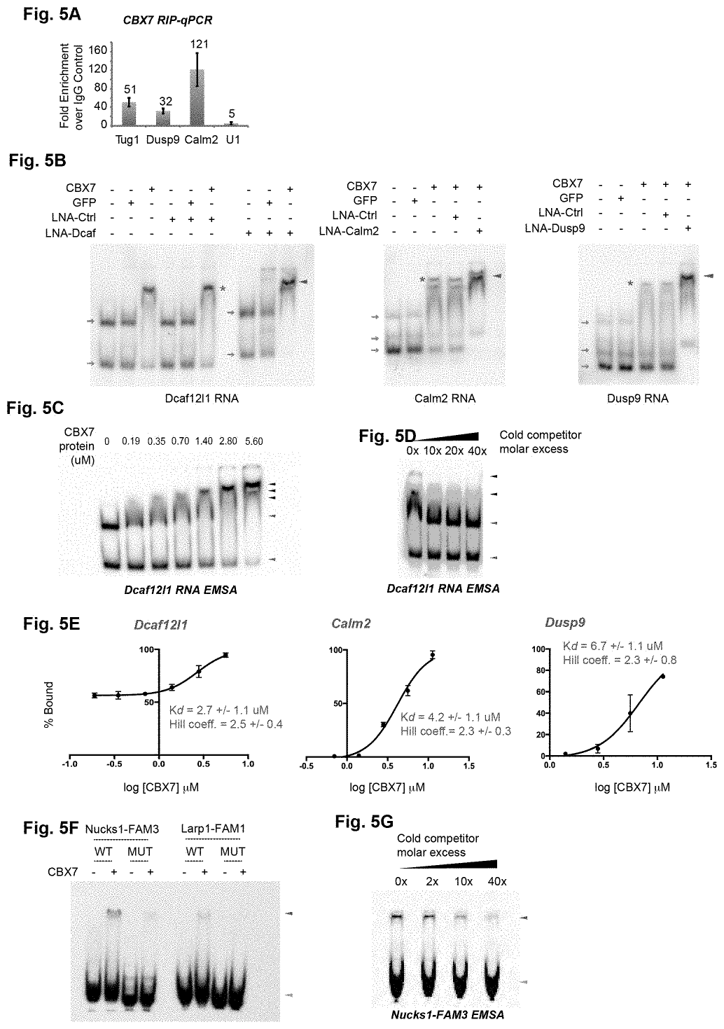

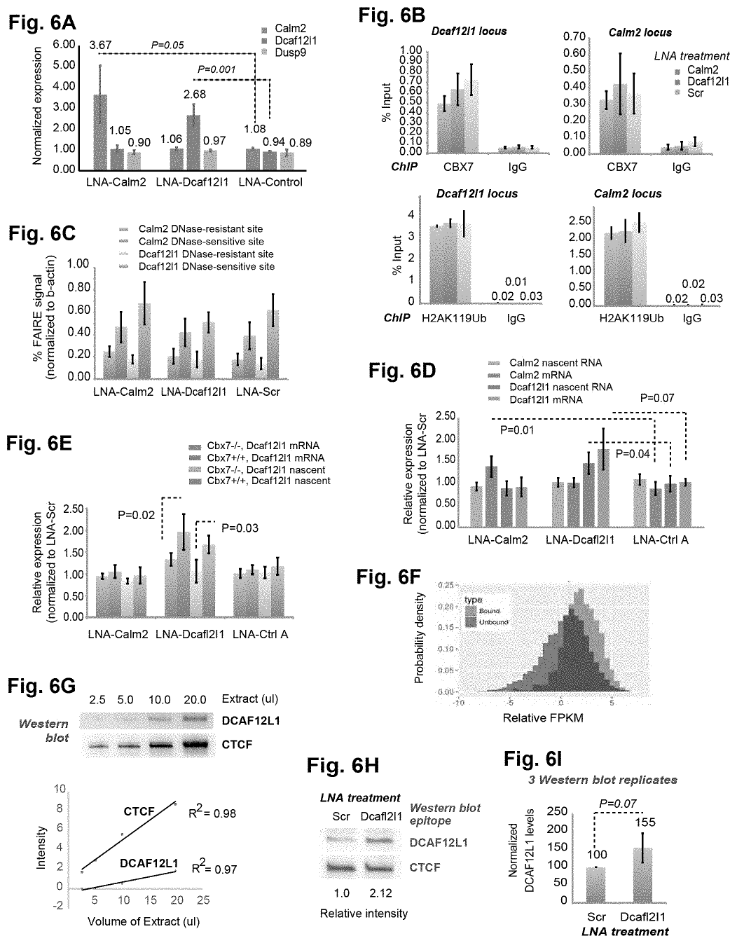

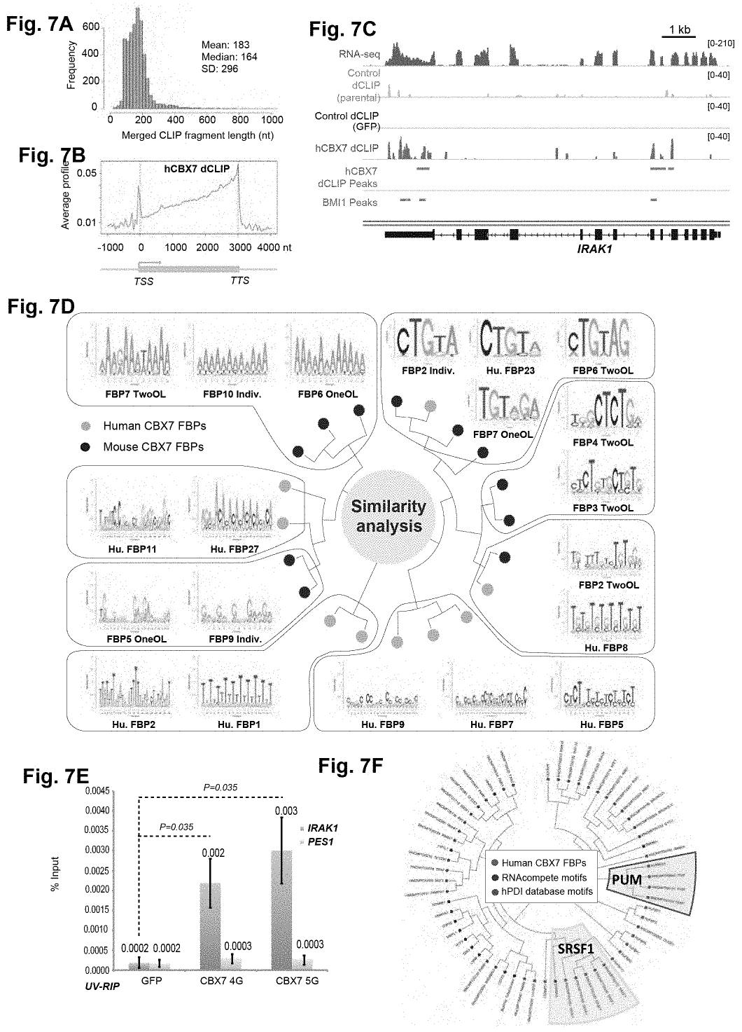

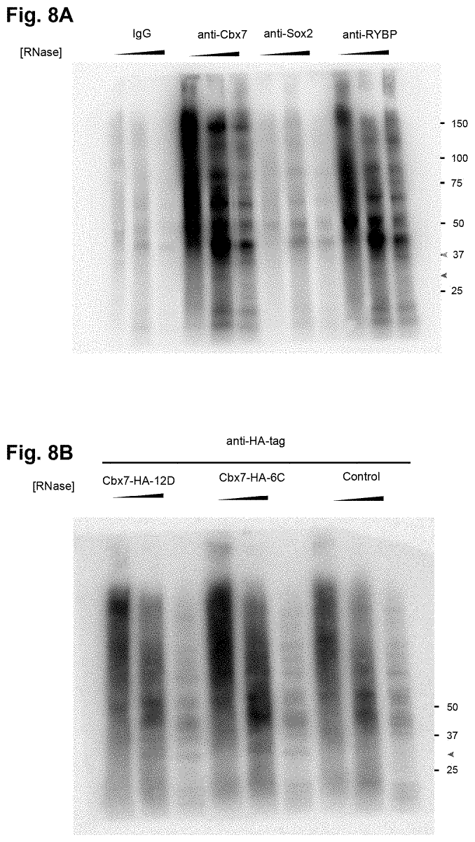

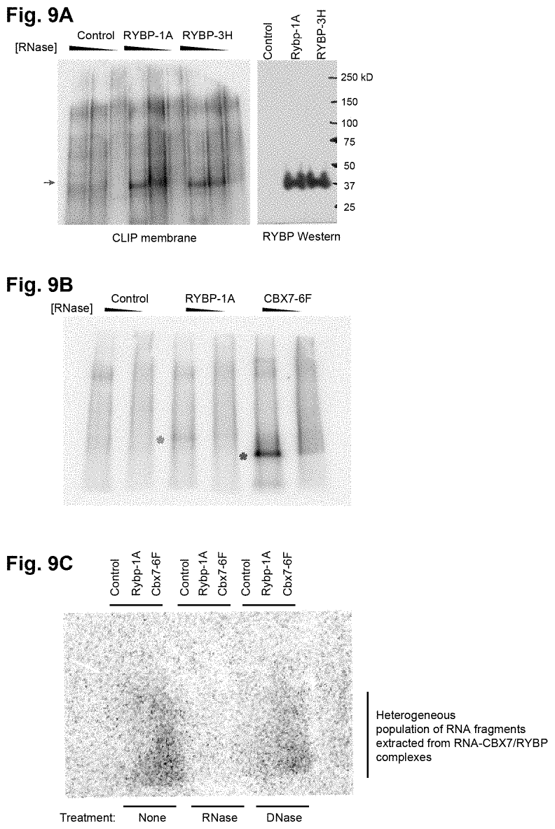

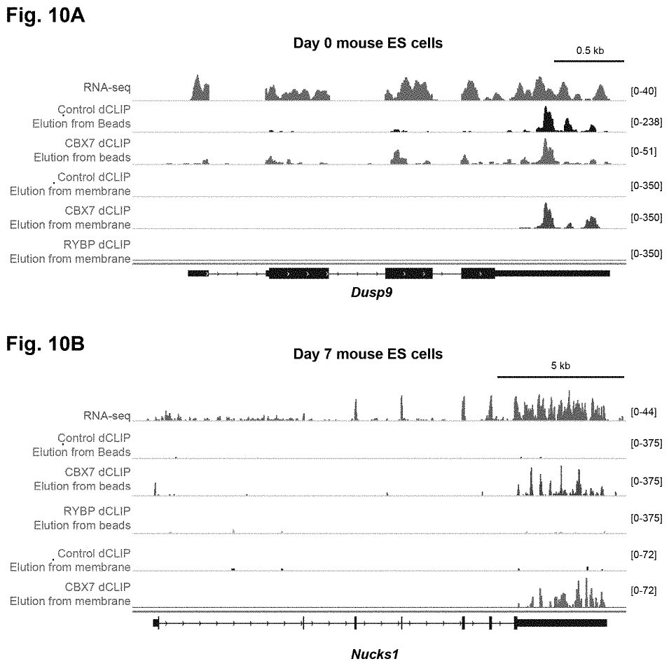

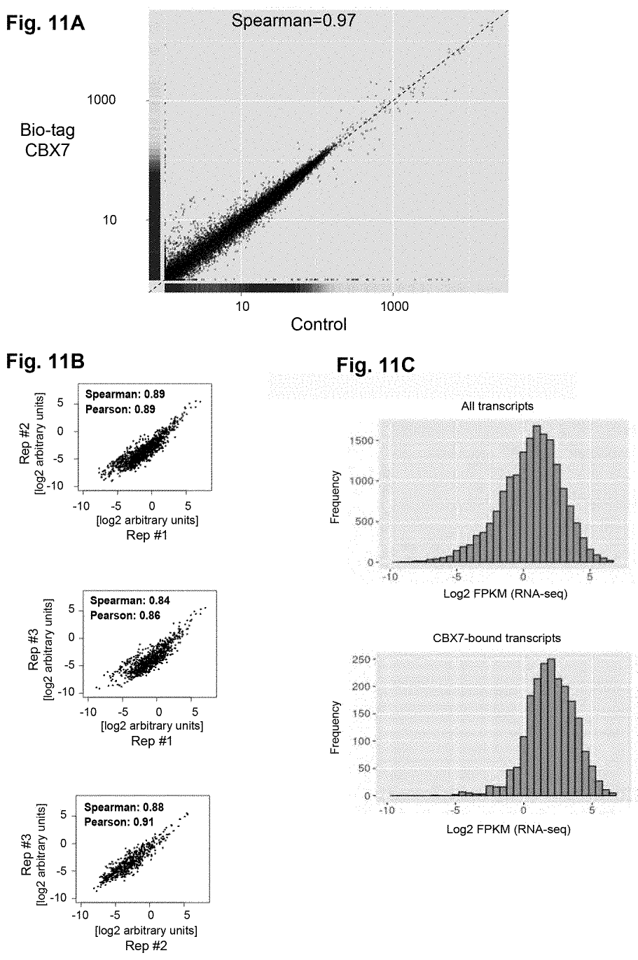

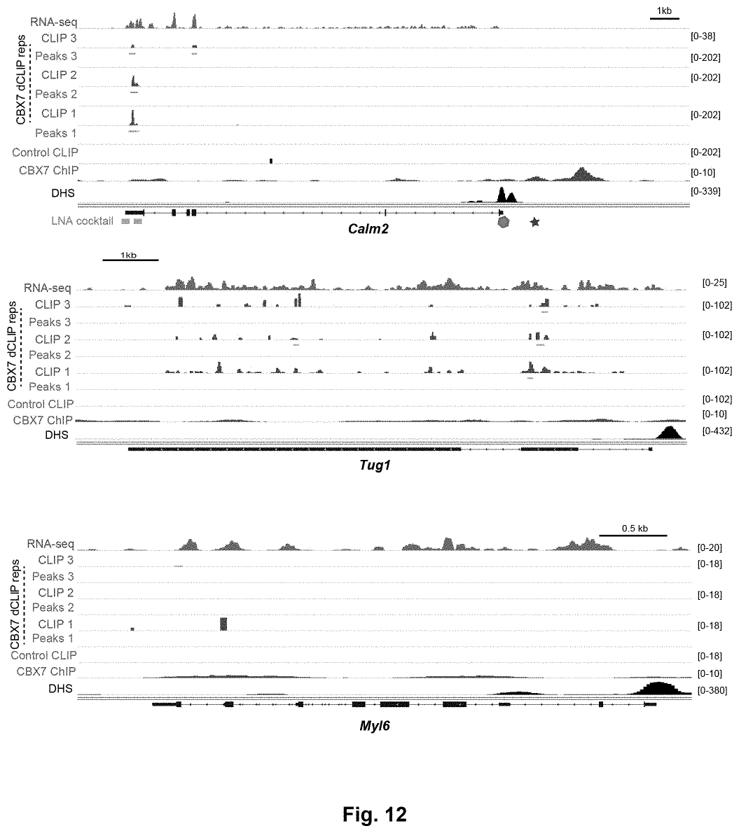

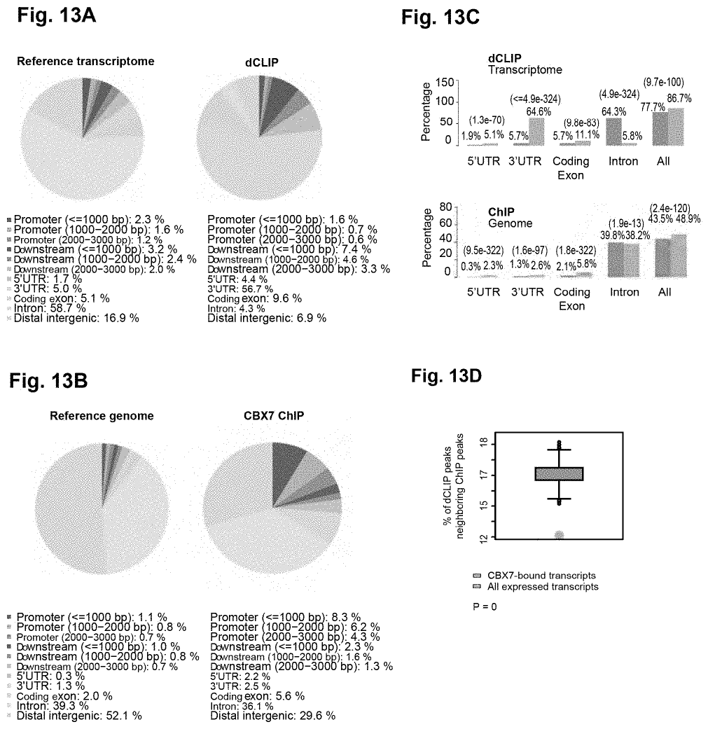

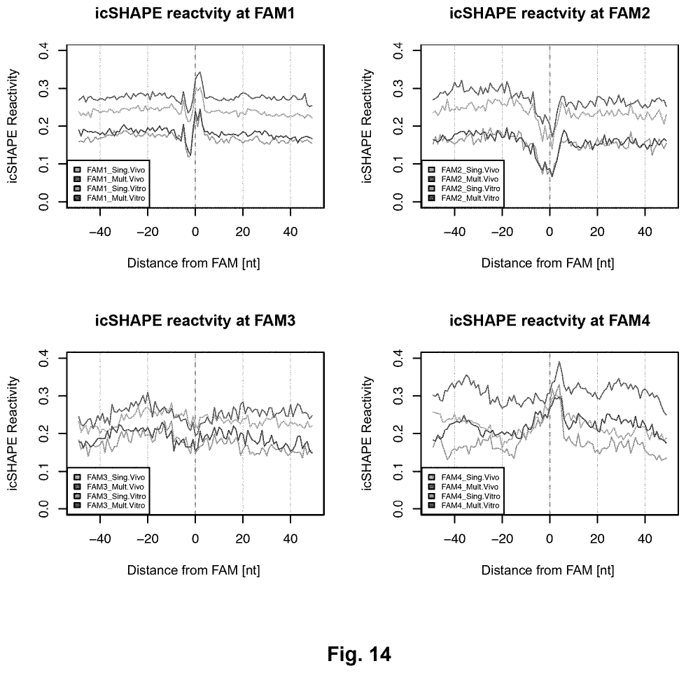

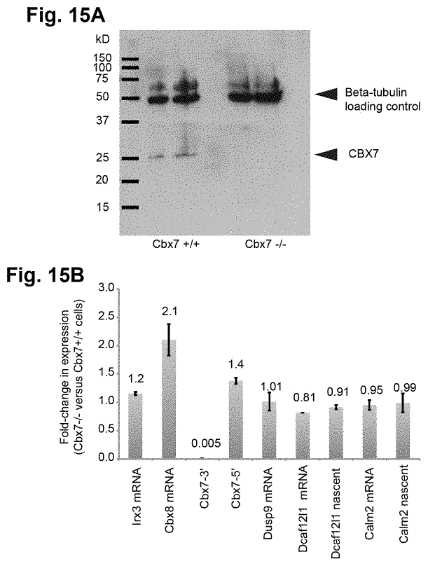

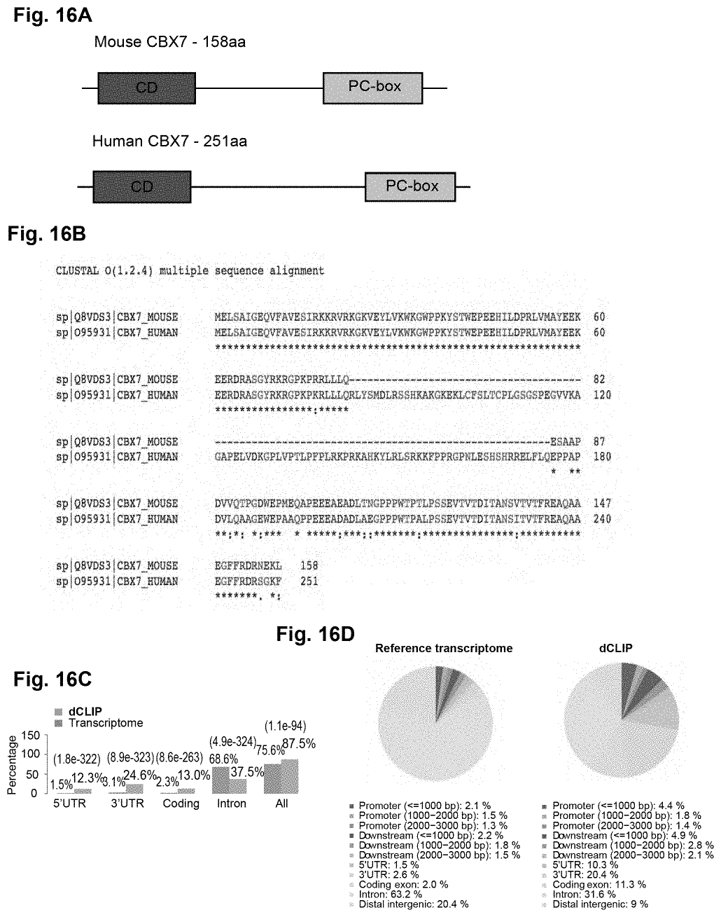

FIGS. 8A-B—Related to FIG. 1. Conventional CLIP of CBX7 and RYBP in ES cells

(Panel A) Representative autoradiography of CLIP experiment using specific antibodies against CBX7 and RYBP. Rabbit IgG and anti-Sox2 antibodies were used as a control. Expected sizes of CBX7 and RYBP proteins were marked by red and green arrowheads respectively. Note a strong background around 40 kDa, which were observed for both CBX7 and RYBP proteins and was not removable up to 1M salt washes as outlined in STAR Methods.

(Panel B) Representative autoradiography of CLIP experiment with anti-HA tag antibody, 6C and 12D are two clonal cell lines expressing physiological levels of HA-tagged-CBX7. Red arrowhead—HA-CBX7 related signal. Note the presence of strong background with anti-HA antibody similar to anti-CBX7 and anti-RYBP CLIP in (A).

FIGS. 9A-C—Related to FIG. 1. Denaturing CLIP of CBX7 and RYBP in ES cells

(Panel A) Representative dCLIP experiment for RYBP protein. Left panel, autoradiography of dCLIP experiment. Right panel, Western blot with anti-RYBP antibody. Red arrows, Biotagged-RYBP signal. 1A and 3H are two clonal cell lines expressing physiological levels of Biotagged-RYBP.

(Panel B) Representative dCLIP experiment performed simultaneously for CBX7 and RYBP proteins. Note a much weaker signal for RYBP (green asterisk) compared to CBX7 (red asterisk).

(Panel C) Radioactively labeled RNA from dCLIP experiment was extracted out of nitrocellulose membrane and subjected to DNAse or RNAse treatment. Subsequently, denaturing PAGE electrophoresis was performed and resulting gel exposed to phosphoimaging screen. Note that radioactive signal was specifically eliminated by RNAse treatment, with DNAse treatment having no visible effect.

FIGS. 10A-B—Related to FIG. 1. Comparison between beads elution and membrane elution dCLIP methods.

Dusp9 RNA dCLIP profile in (Panel A) and Nucks1 RNA dCLIP profile in (Panel B) were examined to assess differences between two RNA extraction methods—elution directly from beads vs. SDS-PAGE purification, nitrocellulose membrane transfer, with elution of RNA from membrane. Note that in both cases, RYBP presented a weaker signal compared to CBX7.

FIGS. 11A-C—Related to FIG. 1. Correlation plots between individual CBX7 dCLIP replicates and overall gene expression.

(Panel A) A scatter plot of gene expression values derived from RNA-seq data of two control lines and two Biotag-CBX7 expressing lines. Note the lack of significant change in overall gene expression. Average FPKM values for endogenous CBX7 expression are 44.38 for control cells versus 46.03 for Biotag-CBX7 cells.

(Panel B) A genome-wide pairwise comparisons of enriched dCLIP peaks over 1 kb bins per three biological replicates (see STAR Methods for details). Note a positive correlation between individual replicates.

(Panel C) Probability density functions for CBX7-bound versus the bulk of expressed transcripts. Relative FPKM values are determined from RNA-seq of the ES cells in which dCLIP was performed. Note that bound transcripts have a tendency towards higher expression.

FIG. 12—Related to FIG. 1. CBX7 binding to target transcripts in mouse ES cells is selective towards a subset of expressed genes

Calm2 (Top Panel) mRNAs represents high binders (green, FIG. 1F). Tug1 (Middle Panel) represents an lncRNA. Notably, while mRNAs prefer to bind CBX7 via the 3′UTR, lncRNAs can bind anywhere within the transcript. Note absent dCLIP signals for Myl6 (Bottom Panel) transcript (negative control), in spite of having comparable expression levels as Calm2.

FIGS. 13A-D—Related to FIG. 1. CEAS analysis for CBX7 dCLIP-seq and ChIP-seq Peaks.

(Panel A) CEAS analysis for CBX7 dCLIP-seq peaks (right pie) with enrichment for each genomic feature shown relative to the overall ES transcriptome profile (left pie).

(Panel B) CEAS analysis for CBX7 ChIP-seq peaks (right pie) with enrichment for each genomic feature shown relative to the overall ES genomic profile (left pie).

(Panel C) Comparison between CBX7 enrichment in distinct genomic features in for CBX7 dCLIP-seq vs CBX7 ChIP-seq.

(Panel D) To assess the relationship between CBX7 binding to RNA vs. DNA, we determined the number of CBX7-bound loci and the number of CBX7-bound transcripts in ES cells. Among the 1,333 transcripts with CBX7 binding sites, only 12% were associated with a CBX7 ChIP peak in the same RefSeq locus, inclusive of promoter region. For bulk expressed transcripts in ES cells, the percentage was significantly greater. To compare the 1,333 transcripts to bulk transcripts, we performed 1,000 rounds of random sampling in each cohort. CBX7 binding to target transcripts is inversely correlated with recruitment of CBX7 to chromatin.

FIG. 14—Related to FIG. 4. Effect of Motif Clustering on RNA secondary structure profile probed in vivo and in vitro on CBX7 RNA binding.

IcSHAPE profiles centered on the genomic sequences predicted as carrying CBX7 binding motifs. IcSHAPE analysis was adopted from Spitale et al (Spitale et al., 2015). Purified RNA molecules were subjected to treatment with icSHAPE reagent in vitro or isolated from cells exposed to icSHAPE reagent in cell culture (in vivo). Extent of RNA folding in particular region is determined by its accessibility to modification by icSHAPE reagent with higher icSHAPE signal representing more open structure. FAM_Sing—depicting single motif per a dCLIP fiber. FAM Mult—depicting multiple motifs per a dCLIP fiber. Note that despite a marked similarity of the curves between single and multiple FAM sequences, average icSHAPE signal in FAM1 and FAM4 is significantly higher than in clustered motif sequences, reflecting more open overall structures.

FIGS. 15A-B—Related to FIG. 6. Gene expression in Cbx7−/− knockout mouse ES cells.

(Panel A) Western blotting with specific CBX7 antibody to confirm the absence of CBX7 protein in the knockout line. Beta-tubulin served as loading control.

(Panel B) Gene expression analysis Cbx7 knockout cells. RT-qPCR experiments with fold-change expression values in Cbx7−/− cells compared to expression in Cbx7+/+ cells. While 5′ region of Cbx7 mRNA was still expressed, the 3′ region was absent, consistent with the knockout scheme described previously (Cheng et al., 2014). Cbx8 is a positive control. Notably, it is known that CBX8 is upregulated in ES cells when CBX7 is depleted, in order to maintain stem cell self-renewal (Morey et al., 2012; O'Loghlen et al., 2012) (FIG. 15B). The functional compensation by CBX8 is consistent with the lack of Dcaf12l1 (Dusp9, Calm2) downregulation in Cbx7−/− cells. Interestingly, however, the gene upregulation effect by the LNA was specific to CBX7. Taken together with FIG. 6E, these data indicate that the mRNA upregulation observed with the gene-specific LNA requires both CBX7 and the gene-specific LNA.

FIGS. 16A-D—Related to FIG. 7. Comparison between Human and Mouse CBX7 isoforms.

(Panel A) Schematic domain structure of CBX7 protein. CD depicted chromodomain, which is involved in binding to methylated lysines and RNA. Note addition of 58aa between CD and PC-box domains in human isoform.

(Panel B) Clustal Omega protein sequence alignment between mouse and human CBX7 isoforms. Note that besides addition of 58aa to human CBX7 in the course of evolution, a very high degree of similarity in CD and PC-box domains still persisted.

(Panel C, Panel D) CEAS analysis for CBX7 CLIP-seq peaks (right pie) with enrichment for each genomic feature shown relative to the overall ES transcriptome profile (left pie).

Table 1—Matrix of FBPs presented in FIGS. 2B and 7D.

### DETAILED DESCRIPTION

While it is now established that many chromatin-modifying complexes interact with RNA (Magistri et al., 2012), a major obstacle in understanding the regulation and function of such interactions has been the difficulty of identifying specific RNA motifs. For instance, interactions between RNA and Polycomb repressive complexes have served as a leading model in our understanding of RNA-protein interactions at the chromatin interface (Khalil et al., 2009; Zhao et al., 2010), but definitive RNA motifs have yet to be identified. Such motifs could exist in the primary RNA sequence or as specific 3D structures. At present, proposed motifs have come from either in vitro binding studies and have yet to be validated in vivo (Wang et al., 2017), or have been deduced from in vivo binding data that yielded whole transcripts or very large footprints of >1 kb (Beltran et al., 2016; Hendrickson et al., 2016; Kaneko et al., 2014a; Kaneko et al., 2014b; Kaneko et al., 2013).

Revealing binding motifs would require a high-fidelity method of generating RNA-binding footprints at a transcriptome-wide level—footprints that represent the protein-binding site on the RNA. While current methodologies have been excellent for highly abundant proteins, including cytoplasmic RNA-binding proteins (Marchese et al., 2016), nuclear epigenetic complexes have presented a greater challenge because of their chromatin association and (hence) a less soluble nature, Such proteins also tend to exist in multi-subunit complexes, with the potential to have several points of contact within a long transcript. New, highly stringent methods that complement existing techniques are therefore much needed in order to obtain a well-rounded view of specific RNA-protein networks.

A major limitation of most existing methodologies is the reliance on antibodies for specific purification of protein-RNA complexes. The relatively low nanomolar affinities of antibody-antigen methods have direct consequences for antibody-based CLIP methods, as they constrain the stringency of washes during the purification step. Because washes must not disrupt the antibody-antigen interaction, nonspecific RNAs cannot be removed efficiently prior to elution. To solve this problem, here we develop “dCLIP” (denaturing CLIP) and provide proof-of-concept in two systems. We show that dCLIP can be applied to both mouse and human CBX7 protein to reveal specific RNA footprints, from which consensus motifs and functionally relevant binding sites can be deduced. We chose the CBX7 subunit of canonical PRC1 for its biological importance. CBX7 is highly expressed in embryonic stem (ES) cells and plays an essential role in maintaining stem cell pluripotency (Morey et al., 2012; O'Loghlen et al., 2012). Existing studies have hinted that CBX7's RNA-binding activity may be critical to its epigenomic function. It is known that CBX7 localization to chromatin depends on its RNA-binding domain, and one RNA (ANRIL) is known to negatively regulate the INK4a locus through CBX7 (Bernstein et al., 2006; Yap et al., 2010). Below we demonstrate that CBX7 interacts with a large family of messenger RNAs (mRNAs), identify short RNA footprints, and develop a bioinformatic pipeline to uncover specific functional motifs.

Here we have developed the denaturing CLIP (dCLIP) methodology and identified a large RNA interactome for CBX7 in human and mouse cells. Interestingly, CBX7 interacts predominantly with mRNA—a somewhat unexpected finding given that previous work with the BMI1 subunit indicated a preference for noncoding RNA (Ray et al., 2016). However, CBX7 is unlike the other CBX isoforms (CBX2, 4, 6, 8) in that it lacks the signature polynucleosome compaction function (Grau et al., 2011). Indeed, our present analysis indicates that CBX7, when associated with the 3′UTR of mRNAs, does not compact or modulate chromatin. Rather, CBX7 is paradoxically associated with a gene upregulatory function. Thus, the RNA-bound CBX7-containing form of PRC1 may not operate as a repressive complex in the same way as PRC1 complexes that contain compaction-competent CBX isoforms. Together, these observations raise the possibility that the immensely heterogeneous PRC1 complexes (as defined by their distinct subunit compositions) may bind different types of transcripts and serve diverse gene regulatory functions, both positive and negative in nature. Recent work with the EZH2 subunit of PRC2 has also revealed direct positive effects on gene regulation (Zovoilis et al., 2016). Thus, although Polycomb proteins have largely been associated with gene-repressive activities, they can serve gene-upregulatory functions in specific instances.

Our current work provides proof-of-concept for the dCLIP methodology. We suggest that dCLIP can complement a number of existing methods, each offering various pro's and con's. A recent popular method is eCLIP (Van Nostrand et al., 2016), which relies on antibody-antigen interactions for RNA precipitation and can be applied to any endogenous protein with good antibodies. Similarly, nRIP and fRIP can also be applied to a wide range of proteins without the need for construction of affinity tags (Hendrickson et al., 2016; Ray et al., 2016; Zhao et al., 2010). These methods have all provided valuable information regarding nuclear RNA-protein networks. What dCLIP offers is a complementary view with certain advantages. One key feature is the highly stringent conditions that enable separation (through denaturation) of tightly associated protein complexes into individual components, which therefore makes possible the assessment of RNA binding activities of a single component within the complex.

Another major advantage of dCLIP method is that it yields a high signal-to-noise ratio and generates reproducible footprints with median sizes of 171 nt (mouse) and 183 nt (human). The small footprints enabled us to identify consensus binding motifs in the RNA that are concordant between two species. We identified families of motifs that tend to co-cluster in the 3′UTR and that share significant similarities between species (mCBX7, hCBX7). While the overall binding affinity of CBX7 to any one FAM is relatively low (Kd in the micromolar range), our data suggest a potential for positive cooperativity that could considerably boost binding dynamics in cells. First, icSHAPE analysis showed that FAM clustering predisposes to an open RNA conformation in vivo (FIG. 14). Second, in vivo CBX7 footprints harboring clustered FAMs demonstrated higher FAM occupancy ratios than those harboring only a single motif (FIG. 3D). Finally, biochemical analysis revealed a Hill coefficient of 2-3 in vitro for three tested 3′UTR examples (FIG. 5E).

The mRNA upregulation following the administration of FAM-targeted LNAs is reminiscent of the RNA-upregulation seen after targeting PRC2-RNA interactions with LNAs against the long noncoding RNA, SMN-AS1, for human spinal muscular atrophy locus (Woo et al., 2017). In the case of SMN-AS1, the LNA blocked PRC2 from binding to the antisense regulatory transcript for SMN2 and thereby prevented the deposition of the repressive H3K27me3 mark. Interestingly, however, chromatin assays suggest that our CBX7-mediated upregulation was not due to reduced levels of the repressive H2AK119Ub mark, nor was it due to increased chromatin accessibility. These findings suggested a co-transcriptional and/or post-transcriptional effect. Indeed, the gene-specific LNAs can increase the steady state levels of both nascent and processed mRNA. Furthermore, Western blot analysis indicated that mRNA upregulation was accompanied by increased protein expression. Potential mechanisms include enhanced transcriptional elongation, RNA splicing, mRNA stability, improved export, or increased translation. One possible hint may come from the paradoxical finding that the mixmer LNAs enhanced (rather than blocked) the CBX7-3′UTR interactions, producing a strong supershift in gel retardation assays. Thus, the binding of CBX7 to the 3′UTR may play a role in transcript stabilization and processing, rather than in chromatin modulation. Notably, our data show that mRNAs bound by CBX7 have a higher probability of expression than transcripts not associated with CBX7 (FIG. 6F). The CBX7-containing form of PRC1 therefore appears to have an activity that has not previously been associated with either canonical or non-canonical PRC1.

Methods of Modulating Gene Expression

The inhibitory nucleic acids and small molecules targeting (e.g., complementary to) a PRC1 binding RNA can be used to modulate gene expression in a cell, e.g., a cancer cell, a stem cell, or other normal cell types for gene or epigenetic therapy. The cells can be in vitro, including ex vivo, or in vivo (e.g., in a subject who has cancer, e.g., a tumor).

In various related aspects, including with respect to the targeting of RNAs by LNA molecule, PRC1-binding RNAs can include endogenous coding and non-coding cellular RNAs, including but not limited to those RNAs that are greater than 60 nt in length, e.g., greater than 100 nt, e.g., greater than 200 nt, have no positive-strand open reading frames greater than 100 amino acids in length, are identified as ncRNAs by experimental evidence, and are distinct from known (smaller) functional-RNA classes (including but not limited to ribosomal, transfer, and small nuclear/nucleolar RNAs, siRNA, piRNA, and miRNA). See, e.g., Lipovich et al., “MacroRNA underdogs in a microRNA world: Evolutionary, regulatory, and biomedical significance of mammalian long non-protein-coding RNA” Biochimica et Biophysica Acta (2010) doi:10.1016/j.bbagrm.2010.10.001; Ponting et al., Cell 136(4):629-641 (2009), Jia et al., RNA 16 (8) (2010) 1478-1487, Dinger et al., Nucleic Acids Res. 37 1685 (2009) D122-D126 (database issue); and references cited therein. ncRNAs have also been referred to as, and can include, long non-coding RNA, long RNA, large RNA, macro RNA, intergenic RNA, and NonCoding Transcripts.

The methods described herein can be used to target both coding and non-coding RNAs. Known classes of RNAs include large intergenic non-coding RNAs (lincRNAs, see, e.g., Guttman et al., Nature. 2009 Mar. 12; 458(7235):223-7. Epub 2009 Feb. 1, which describes over a thousand exemplary highly conserved large non-coding RNAs in mammals; and Khalil et al., PNAS 106(28)11675-11680 (2009)); promoter associated short RNAs (PASRs; see, e.g., Seila et al., Science. 2008 Dec. 19; 322(5909):1849-51. Epub 2008 Dec. 4; Kanhere et al., Molecular Cell 38, 675-688, (2010)); endogenous antisense RNAs (see, e.g., Numata et al., BMC Genomics. 10:392 (2009); Okada et al., Hum Mol Genet. 17(11):1631-40 (2008); Numata et al., Gene 392(1-2):134-141 (2007); and Rosok and Sioud, Nat Biotechnol. 22(1):104-8 (2004)); and RNAs that bind chromatin modifiers such as PRC2 and LSD1 (see, e.g., Tsai et al., Science. 2010 Aug. 6; 329(5992):689-93. Epub 2010 Jul. 8; and Zhao et al., Science. 2008 Oct. 31; 322(5902):750-6).

Exemplary ncRNAs include XIST, TSIX, SRA1, and KCNQ1OT1. The sequences for more than 17,000 long human ncRNAs can be found in the NCode™ Long ncRNA Database on the Invitrogen website. Additional long ncRNAs can be identified using, e.g., manual published literature, Functional Annotation of Mouse (FANTOM3) project, Human Full-length cDNA Annotation Invitational (H-Invitational) project, antisense ncRNAs from cDNA and EST database for mouse and human using a computation pipeline (Zhang et al., Nucl. Acids Res. 35 (suppl 1): D156-D161 (2006); Engstrom et al., PLoS Genet. 2:e47 (2006)), human snoRNAs and scaRNAs derived from snoRNA-LBME-db, RNAz (Washietl et al. 2005), Noncoding RNA Search (Torarinsson, et al. 2006), and EvoFold (Pedersen et al. 2006).

A transcriptome of exemplary PRC1-binding RNAs that can be targeted with the present methods is described in WO 2016/149455, which is incorporated by reference herein in its entirety. See, e.g., Table 1 of WO 2016/149455: Human CBX7-RNA binding sites as determined by denaturing CLIP-seq analysis in Human 293 cells. All coordinates in hg19. The columns (c) correspond to: c1, SEQ ID Number. c2, Chromosome number. c3, Read start position. c4, Read end position. c5, chromosome strand that the transcript is made from (+, top or Watson strand; −, bottom or Crick strand of each chromosome). C6, nearest gene name. c7, gene categories as defined in Example 2.

See also Table 2 of WO 2016/149455: Human LiftOver sequences corresponding to CBX7-RNA binding sites as determined by denaturing CLIP-seq analysis in mouse ES cells shown. All coordinates in hg19. CBX7-binding sites derived from CLIP-seq performed in the mouse ES cell line, 16.7, as shown in Table 3, are translated from mouse mm9 to human hg19 coordinates.

In addition, see Table 3 of WO 2016/149455: Mouse CBX7-RNA binding sites as determined by denaturing CLIP-seq analysis in ES cells derived from Mus musculus. All coordinates in mm9. CLIP-seq performed in the mouse ES cell line, EL 16.7. CBX7 binding sites in the RNA are shown.

Calculations of homology or sequence identity between sequences (the terms are used interchangeably herein) are performed as follows.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

The methods described herein can be used for modulating expression of oncogenes and tumor suppressors in cells, e.g., cancer cells. For example, to decrease expression of an gene (e.g., an oncogene or imprinted gene) in a cell, the methods include introducing into the cell an inhibitory nucleic acid or small molecule that specifically binds, or is complementary, to a PRC1-binding region of an RNA that increases expression of the gene, e.g., an oncogene and/or an imprinted gene, set forth in Tables 1-3. As another example, to increase expression of a gene, e.g., a tumor suppressor, in a cell, the methods include introducing into the cell an inhibitory nucleic acid or small molecule that specifically binds, or is complementary, to a PRC1-binding region of an RNA that decreases expression of the gene, e.g., of a tumor suppressor gene, set forth in Tables 1-3, e.g., in subjects with cancer, e.g., lung adenocarcinoma patients.

In general, the methods include introducing into the cell an inhibitory nucleic acid that specifically binds, or is complementary, to a region of an RNA that modulated expression of a gene as set forth in Tables 1-3.

In preferred embodiments, the inhibitory nucleic acid binds to a region within or near (e.g., within 100, 200, 300, 400, 500, 600, 700, 1K, 2K, or 5K bases of) a PRC1-binding region of the RNA as set forth in Tables 1-3. The empirically-identified “peaks,” which are believed to represent PRC1-binding regions are shown in Table 1, with 500 nts of sequence on each side, so that in some the methods can include targeting a sequence as shown in one of the sequences in Tables 1-3, or a sequence that is between 500 nts from the start and 500 nts of the end of a sequence shown in Tables 1-3, or between 400 nts from the start and 400 nts of the end, 300 nts from the start and 300 nts of the end, between 200 nts from the start and 200 nts of the end, or between 100 nts from the start and 100 nts of the end, of a sequence shown in Tables 1-3. A nucleic acid that binds “specifically” binds primarily to the target RNA or related RNAs to inhibit regulatory function of the RNA but not of other non-target RNAs. The specificity of the nucleic acid interaction thus refers to its function (e.g., inhibiting the PRC1-associated repression of gene expression) rather than its hybridization capacity. Inhibitory nucleic acids may exhibit nonspecific binding to other sites in the genome or other RNAs, without interfering with binding of other regulatory proteins and without causing degradation of the non-specifically-bound RNA. Thus this nonspecific binding does not significantly affect function of other non-target RNAs and results in no significant adverse effects.

These methods can be used to treat a cancer in a subject by administering to the subject a composition (e.g., as described herein) comprising a PRC1-binding fragment of an RNA as described herein and/or an inhibitory nucleic acid that binds to an RNA (e.g., an inhibitory nucleic acid that binds to an RNA that inhibits a tumor suppressor, or cancer-suppressing gene, or imprinted gene and/or other growth-suppressing genes in any of Tables 1-3). Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease. With respect to cancer, treating includes inhibiting tumor cell proliferation, increasing tumor cell death or killing, inhibiting rate of tumor cell growth or metastasis, reducing size of tumors, reducing number of tumors, reducing number of metastases, increasing 1-year or 5-year survival rate.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung (e.g. small cell, non-small cell, squamous, adenocarcinoma), breast, thyroid, lymphoid, gastrointestinal, genito-urinary tract, kidney, bladder, liver (e.g. hepatocellular cancer), pancreas, ovary, cervix, endometrium, uterine, prostate, brain, as well as adenocarcinomas which include malignancies such as most colon cancers, colorectal cancer, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, specific cancers that can be treated using the methods described herein include, but are not limited to: breast, lung, prostate, CNS (e.g., glioma), salivary gland, prostate, ovarian, and leukemias (e.g., ALL, CML, or AML). Associations of these genes with a particular cancer are known in the art, e.g., as described in Futreal et al., Nat Rev Cancer. 2004; 4;177-83; and The COSMIC (Catalogue of Somatic Mutations in Cancer) database and website, Bamford et al., Br J Cancer. 2004; 91;355-8; see also Forbes et al., Curr Protoc Hum Genet. 2008; Chapter 10; Unit 10.11, and the COSMIC database, e.g., v.50 (Nov. 30, 2010).

In addition, the methods described herein can be used for modulating (e.g., enhancing or decreasing) pluripotency of a stem cell and to direct stem cells down specific differentiation pathways to make endoderm, mesoderm, ectoderm, and their developmental derivatives. To increase, maintain, or enhance pluripotency, the methods include introducing into the cell an inhibitory nucleic acid that specifically binds to, or is complementary to, a motif as described herein within a PRC1-binding site on a non-coding RNA as set forth in SEQ ID NOs:1 to 5893 (human), 5894 to 17415 (human), and 17416 to 36368 (mouse) or in any of Tables 1-3 of WO 2016/149455. Stem cells useful in the methods described herein include adult stem cells (e.g., adult stem cells obtained from the inner ear, bone marrow, mesenchyme, skin, fat, liver, muscle, or blood of a subject, e.g., the subject to be treated); embryonic stem cells, or stem cells obtained from a placenta or umbilical cord; progenitor cells (e.g., progenitor cells derived from the inner ear, bone marrow, mesenchyme, skin, fat, liver, muscle, or blood); and induced pluripotent stem cells (e.g., iPS cells).

Furthermore, the present methods can be used to treat Systemic Lupus erythematosus (SLE), an autoimmune disease that affects 1.5 million Americans (16,000 new cases per year). Ages 10-50 are the most affected, with more sufferers being female than male. SLE is a multi-organ disease; the effects include arthritis, joint pain & swelling, chest pain, fatigue, general malaise, hair loss, mouth sores, sensitivity to light, skin rash, and swollen lymph nodes. Current treatments include corticosteroids, immunosuppressants, and more recently belimumab (an inhibitor of B cell activating factor).

The causes of SLE are probably multiple, including HLA haplotypes. The interleukin 1 receptor associated kinase 1 (IRAK1) has been implicated in some patients. IRAK1 is X-linked (possibly explaining the female predominance of the disease) and is involved in immune response to foreign antigens and pathogens. IRAK1 has been associated with SLE in both adult and pediatric forms. Overexpression of IRAK1 in animal models causes SLE, and knocking out IRAK1 in mice alleviates symptoms of SLE. See, e.g., Jacob et al., Proc Natl Acad Sci USA. 2009 Apr. 14; 106(15):6256-61. The present methods can include treating a subject with SLE by administering an inhibitory nucleic acid that is complementary to a PRC1-binding region on IRAK1 RNA, e.g., an LNA targeting the 3′ UTR as shown in FIGS. 2D and 5B, e.g., as shown in Table 4.

The present methods can also be used to treat MECP2 Duplication Syndrome in a subject. This condition is characterized by mental retardation, weak muscle tone, and feeding difficulties, as well as poor/absent speech, seizures, and muscle spasticity. There are more reported cases in males than in females; female carriers may have skewed XCI. There is a 50% mortality rate by age 25 associated with this condition, which accounts for 1-2% of X-linked mental retardation. The real rate of incidence is unknown, as many go undiagnosed. Genetically, the cause is duplication (even triplication) of MECP2 gene. There is no current treatment. The present methods can include treating a subject with MECP2 Duplication Syndrome by administering an inhibitory nucleic acid that is complementary to a motif as described herein within a PRC1-binding region on Mecp2 RNA, e.g., an LNA targeting the 3′UTR of Mecp2 as shown in FIGS. 2C and 5A of WO 2016/149455, e.g., as shown in Table 4 of WO 2016/149455.

In some embodiments, the methods described herein include administering a composition, e.g., a sterile composition, comprising an inhibitory nucleic acid that is complementary to a motif as described herein within a PRC1-binding region on an RNA, e.g., as set forth in SEQ ID NOs:1 to 5893 (human), 5894 to 17415 (human), and 17416 to 36368 (mouse) or in any of Tables 1-3 of WO 2016/149455. Inhibitory nucleic acids for use in practicing the methods described herein can be an antisense or small interfering RNA, including but not limited to an shRNA or siRNA. In some embodiments, the inhibitory nucleic acid is a modified nucleic acid polymer (e.g., a locked nucleic acid (LNA) molecule).

Inhibitory nucleic acids have been employed as therapeutic moieties in the treatment of disease states in animals, including humans. Inhibitory nucleic acids can be useful therapeutic modalities that can be configured to be useful in treatment regimes for the treatment of cells, tissues and animals, especially humans.

For therapeutics, an animal, preferably a human, suspected of having cancer is treated by administering an RNA or inhibitory nucleic acid in accordance with this invention. For example, in one non-limiting embodiment, the methods comprise the step of administering to the animal in need of treatment, a therapeutically effective amount of an RNA or inhibitory nucleic acid as described herein.

Inhibitory Nucleic Acids

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In the present methods, the inhibitory nucleic acids are preferably designed to target a motif as described herein within a region of the RNA that binds to PRC1, e.g., as described in WO 2016/149455 (see Tables 1-3 thereof). The motifs are shown in FIG. 2 and FIG. 7D and in the matrices shown in Table 1. In some embodiments, the motifs comprise the “consensus” sequences shown in Table 1. In some embodiments, the motifs are constructed using the top 1, top 2, or top 3 nucleotides at each position. In some embodiments, the motifs are constructed using the nucleotides present in greater than 0.1, 0.2, 0.3, or 0.4 of the target sequences, using the percentages as shown in Table 1.

These “inhibitory” nucleic acids are believed to work by inhibiting the interaction between the RNA and PRC1, and as described herein can be used to modulate expression of a gene.

In some embodiments, the inhibitory nucleic acids are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such inhibitory nucleic acids; for example, an inhibitory nucleic acid 30 nucleotides in length may have a portion of 15 bases that is complementary to the targeted RNA. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the inhibitory nucleic acid comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Additional modifications are possible as described in WO 2016/149455.

The inhibitory nucleic acids useful in the present methods are sufficiently complementary to the target RNA, e.g., hybridize sufficiently well and with sufficient biological functional specificity, to give the desired effect. “Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of an inhibitory nucleic acid is capable of hydrogen bonding with a base at the corresponding position of an RNA, then the bases are considered to be complementary to each other at that position. 100% complementarity is not required. As noted above, inhibitory nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

In some embodiments, the location on a target RNA to which an inhibitory nucleic acids hybridizes is defined as a region to which a protein binding partner binds, as shown in Tables 1-3. Routine methods can be used to design an inhibitory nucleic acid that binds to this sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, methods of designing oligonucleotides similar to the inhibitory nucleic acids described herein, and various options for modified chemistries or formats, are exemplified in Lennox and Behlke, Gene Therapy (2011) 18: 1111-1120, which is incorporated herein by reference in its entirety, with the understanding that the present disclosure does not target miRNA ‘seed regions’.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. Target segments 5-500 nucleotides in length comprising a stretch of at least five (5) consecutive nucleotides within the protein binding region, or immediately adjacent thereto, are considered to be suitable for targeting as well. Target segments can include sequences that comprise at least the 5 consecutive nucleotides from the 5′-terminus of one of the protein binding regions (the remaining nucleotides being a consecutive stretch of the same RNA beginning immediately upstream of the 5′-terminus of the binding segment and continuing until the inhibitory nucleic acid contains about 5 to about 100 nucleotides). Similarly preferred target segments are represented by RNA sequences that comprise at least the 5 consecutive nucleotides from the 3′-terminus of one of the illustrative preferred target segments (the remaining nucleotides being a consecutive stretch of the same RNA beginning immediately downstream of the 3′-terminus of the target segment and continuing until the inhibitory nucleic acid contains about 5 to about 100 nucleotides). One having skill in the art armed with the sequences provided herein will be able, without undue experimentation, to identify further preferred protein binding regions to target with complementary inhibitory nucleic acids.

In the context of the present disclosure, hybridization means base stacking and hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as the term is used in the art, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides that can hydrogen bond with each other through their bases. Thus, “specifically hybridizable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridizable when binding of the sequence to the target RNA molecule interferes with the normal function of the target RNA to cause a loss of activity (e.g., inhibiting PRC1-associated repression with consequent up-regulation of gene expression) and there is a sufficient degree of complementarity to avoid non-specific binding of the sequence to non-target RNA sequences under conditions in which avoidance of the non-specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the invention that hybridize to an RNA are identified through routine experimentation. In general the inhibitory nucleic acids must retain specificity for their target, i.e., either do not directly bind to, or do not directly significantly affect expression levels of, transcripts other than the intended target.

Target-specific effects, with corresponding target-specific functional biological effects, are possible even when the inhibitory nucleic acid exhibits non-specific binding to a large number of non-target RNAs. For example, short 8 base long inhibitory nucleic acids that are fully complementary to a RNA may have multiple 100% matches to hundreds of sequences in the genome, yet may produce target-specific effects, e.g. upregulation of a specific target gene through inhibition of PRC1 activity. 8-base inhibitory nucleic acids have been reported to prevent exon skipping with with a high degree of specificity and reduced off-target effect. See Singh et al., RNA Biol., 2009; 6(3): 341-350. 8-base inhibitory nucleic acids have been reported to interfere with miRNA activity without significant off-target effects. See Obad et al., Nature Genetics, 2011; 43: 371-378.

For further disclosure regarding inhibitory nucleic acids, please see WO 2016/149455 as well as US2010/0317718 (antisense oligos); US2010/0249052 (double-stranded ribonucleic acid (dsRNA)); US2009/0181914 and US2010/0234451 (LNA molecules); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA in vitro, and are expected to inhibit the activity of PRC1 in vivo. Thus, oligonucleotides are chosen that are sufficiently complementary to the target, i.e., that hybridize sufficiently well and with sufficient biological functional specificity, to give the desired effect.

Modified Base, Including Locked Nucleic Acids (LNAs)

In some embodiments, the inhibitory nucleic acids used in the methods described herein comprise one or more modified bonds or bases. Modified bases include phosphorothioate, methylphosphonate, peptide nucleic acids, or locked nucleic acids (LNAs). Preferably, the modified nucleotides are part of locked nucleic acid molecules, including [alpha]-L-LNAs. LNAs include ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The modified base/LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The modified base/LNA molecules can be chemically synthesized using methods known in the art.

The modified base/LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of a modified base/LNA molecule; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing modified base/LNA molecules are known in the art; for example, LNA sequences will bind very tightly to other LNA sequences, so it is preferable to avoid significant complementarity within an LNA molecule. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNA molecules see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jensen et al., Oligonucleotides 14:130-146 (2004); Kauppinen et al., Drug Disc. Today 2(3):287-290 (2005); and Ponting et al., Cell 136(4):629-641 (2009), and references cited therein.

As demonstrated herein and previously (see, e.g., WO 2012/065143 and WO 2012/087983, incorporated herein by reference), LNA molecules can be used as a valuable tool to manipulate and aid analysis of RNAs. Advantages offered by an LNA molecule-based system are the relatively low costs, easy delivery, and rapid action. While other inhibitory nucleic acids may exhibit effects after longer periods of time, LNA molecules exhibit effects that are more rapid, e.g., a comparatively early onset of activity, are fully reversible after a recovery period following the synthesis of new RNA, and occur without causing substantial or substantially complete RNA cleavage or degradation. One or more of these design properties may be desired properties of the inhibitory nucleic acids of the invention. Additionally, LNA molecules make possible the systematic targeting of domains within much longer nuclear transcripts. Although a PNA-based system has been described earlier, the effects on Xi were apparent only after 24 hours (Beletskii et al., Proc Natl Acad Sci USA. 2001; 98:9215-9220). The LNA technology enables high-throughput screens for functional analysis of non-coding RNAs and also provides a novel tool to manipulate chromatin states in vivo for therapeutic applications.

In various related aspects, the methods described herein include using LNA molecules to target RNAs for a number of uses, including as a research tool to probe the function of a specific RNA, e.g., in vitro or in vivo. The methods include selecting one or more desired RNAs, designing one or more LNA molecules that target the RNA, providing the designed LNA molecule, and administering the LNA molecule to a cell or animal. The methods can optionally include selecting a region of the RNA and designing one or more LNA molecules that target that region of the RNA.

Aberrant imprinted gene expression is implicated in several diseases including Long QT syndrome, Beckwith-Wiedemann, Prader-Willi, and Angelman syndromes, as well as behavioral disorders and carcinogenesis (see, e.g., Falls et al., Am. J. Pathol. 154:635-647 (1999); Lalande, Annu Rev Genet 30:173-195 (1996); Hall Annu Rev Med. 48:35-44 (1997)). LNA molecules can be created to treat such imprinted diseases. As one example, the long QT Syndrome can be caused by a K+ gated Calcium-channel encoded by Kcnq1. This gene is regulated by its antisense counterpart, the long noncoding RNA, Kcnq1ot1 (Pandey et al., Mol Cell. 2008 Oct. 24; 32(2):232-46). Disease arises when Kcnq1ot1 is aberrantly expressed. LNA molecules can be created to downregulate Kcnq1ot1, thereby restoring expression of Kcnq1. As another example, LNA molecules could inhibit RNA cofactors for polycomb complex chromatin modifiers to reverse the imprinted defect.

From a commercial and clinical perspective, the timepoints between about 1 to 24 hours potentially define a window for epigenetic reprogramming. The advantage of the LNA system is that it works quickly, with a defined half-life, and is therefore reversible upon degradation of LNAs, at the same time that it provides a discrete timeframe during which epigenetic manipulations can be made. By targeting nuclear long RNAs, LNA molecules or similar polymers, e.g., xylo-LNAs, might be utilized to manipulate the chromatin state of cells in culture or in vivo, by transiently eliminating the regulatory RNA and associated proteins long enough to alter the underlying locus for therapeutic purposes. In particular, LNA molecules or similar polymers that specifically bind to, or are complementary to, PRC1-binding RNA can prevent recruitment of PRC1 to a specific chromosomal locus, in a gene-specific fashion.

LNA molecules might also be administered in vivo to treat other human diseases, such as but not limited to cancer, neurological disorders, infections, inflammation, and myotonic dystrophy. For example, LNA molecules might be delivered to tumor cells to downregulate the biologic activity of a growth-promoting or oncogenic long nuclear RNA (e.g., Gtl2 or MALAT1 (Luo et al., Hepatology. 44(4):1012-24 (2006)), a RNA associated with metastasis and is frequently upregulated in cancers). Repressive RNAs downregulating tumor suppressors can also be targeted by LNA molecules to promote reexpression. For example, expression of the INK4b/ARF/INK4a tumor suppressor locus is controlled by Polycomb group proteins including PRC1 and PRC1 and repressed by the antisense noncoding RNA ANRIL (Yap et al., Mol Cell. 2010 Jun. 11; 38(5):662-74). PRC1-binding regions described herein in ANRIL can be targeted by LNA molecules to promote reexpression of the INK4b/ARF/INK4a tumor suppressor. Some ncRNAs may be positive regulators of oncogenes. Such “activating ncRNAs” have been described recently (e.g., Jpx (Tian et al., Cell. 143(3):390-403 (2010) and others (Ørom et al., Cell. 143(1):46-58 (2010)). Therefore, LNA molecules could be directed at these activating ncRNAs to downregulate oncogenes. LNA molecules could also be delivered to inflammatory cells to downregulate regulatory ncRNA that modulate the inflammatory or immune response. (e.g., LincRNA-Cox2, see Guttman et al., Nature. 458(7235):223-7. Epub 2009 Feb. 1 (2009)).

In still other related aspects, the LNA molecules targeting PRC1-binding regions in RNAs described herein can be used to create animal or cell models of conditions associated with altered gene expression (e.g., as a result of altered epigenetics).

The methods described herein may also be useful for creating animal or cell models of other conditions associated with aberrant imprinted gene expression, e.g., as noted above.

In various related aspects, the results described herein demonstrate the utility of LNA molecules for targeting RNA, for example, to transiently disrupt chromatin for purposes of reprogramming chromatin states ex vivo. Because LNA molecules stably displace RNA for hours and chromatin does not rebuild for hours thereafter, LNA molecules create a window of opportunity to manipulate the epigenetic state of specific loci ex vivo, e.g., for reprogramming of hiPS and hESC prior to stem cell therapy. For example, Gtl2 controls expression of DLK1, which modulates the pluripotency of iPS cells. Low Gtl2 and high DLK1 is correlated with increased pluripotency and stability in human iPS cells. Thus, LNA molecules targeting Gtl2 can be used to inhibit differentiation and increase pluripotency and stability of iPS cells.

See also PCT/US11/60493, which is incorporated by reference herein in its entirety.

Interfering RNA, Including siRNA/shRNA

In some embodiments, the inhibitory nucleic acid sequence that is complementary to an RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

In some embodiments, the inhibitory nucleic acids are ribozymes. Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261 :1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 MM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. If desired, nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)).

Preferably, inhibitory nucleic acids of the invention are synthesized chemically. Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066; WO/2008/043753 and WO/2008/049085, and the refences cited therein.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

It is understood that any of the modified chemistries or formats of inhibitory nucleic acids described herein can be combined with each other, and that one, two, three, four, five, or more different types of modifications can be included within the same molecule.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Pharmaceutical Compositions

The methods described herein can include the administration of pharmaceutical compositions and formulations comprising inhibitory nucleic acid sequences designed to target an RNA.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krützfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acid molecules (“LNA molecules”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

### EXAMPLES

The invention is further described in the following examples, which do not limit the scope of the invention described in the claims.

Materials and Methods

The following materials and methods were used in the Examples set forth below.

Experimental Model and Subject Details

EL 16.7 (129/Cas) mouse female embryonic stem cells were described previously (Lee and Lu, 1999). Cbx7+/+ and Cbx7−/− mouse embryonic stem cell lines were generated by Dr. Bo Cheng in the laboratory of Dr. T. Kerppola (University of Michigan) as described in (Cheng et al., 2014), and kindly provided by Dr. Xiaojun Ren (University of Colorado). All stem cell lines were routinely maintained in 500 U/ml LIF, DME, and 15% FCS on gamma-irradiated mouse embryonic fibroblasts feeder layer. For differentiation, 7×10⁵ cells were plated on pre-gelatinized 150 mm TC plates and grown in monolayer for 7 days in DME+15% FBS without LIF. HEK293 cells were routinely maintained in DME+10% FBS.

Stable Transfection

The following plasmid vectors were used for stable transfection into EL16.7 ES cells:

pCAGGS—mouse CBX7-Flag-HA-IRES-Puro-GFP plasmid was used for stable expression of HA-tagged CBX7 for ChIP-seq experiments. pCAGGS-IRES-Puro-GFP plasmid was a kind gift from Dr. Mitinori Saitou (Kyoto University, Japan).

pEF1aBirAV5His plasmid was utilized for stable expression of V5-His-tagged BirA bacterial biotinylase in EL16.7 ES cells.

pEF1a-Flag-biotag-PGKpuro-mCBX7 and pEF1a-Flag-biotag-PGKpuro-mRYBP plasmids were employed for stable transfection of mouse CBX7 and RYBP carrying biotinylation tag in EL16.7 cells expressing BirA biotinylase.

pCAG-Avi-GFP-hCBX7-IRES-Puro plasmid was employed for stable transfection of human CBX7 carrying biotinylation tag in HEK293 cells expressing BirA biotinylase.

pEF1aBirAV5His and pEF1-Flag-Biotag plasmid vectors were a kind gift from Dr. Stuart Orkin (Harvard Medical School) and have been described previously by Kim et al (Kim et al., 2009).

pCAG-Avi-GFP-IRES-Puro plasmid was a kind gift from Dr. Mitinori Saitou, Department of Anatomy and Cell Biology, Graduate School of Medicine, Kyoto University.

To create mouse ES cells with stable expression of recombinant proteins, EL 16.7 mouse ES cells were grown to 70% confluence on embryonic feeder layer in T75 flasks. Cells were trypsinized and 2×10⁷ cells electroporated with 30 μg of linearized vector in PBS using GenePulser II (Bio-Rad). Positive cells were selected using growth media supplemented with 1 μg/ml Puromycin (Gibco) alone or in combination with 300 μg/ml G418. To create HEK293 cells with stable expression of recombinant proteins, cells were grown to 70% confluence in T75 flasks. Cells were trypsinized and 1×10⁷ cells electroporated with 15 μg of linearized vector in PBS using GenePulser II (Bio-Rad). Positive cells were selected using growth media supplemented with 1 μg/ml Puromycin (Gibco) alone or in combination with 300 μg/ml G418. Stable transfection and expression of recombinant proteins was confirmed by PCR genotyping and Western blotting with specific antibodies.

CLIP Method—Small Scale

The conventional CLIP method was performed as described previously (Jeon and Lee, 2011). Cells were grown to full confluence in 15 cm tissue culture dishes. Medium was then aspirated and cells were washed with 10 ml ice-cold phosphate-buffered saline (PBS) (containing 8.1 mM Na₂HPO₄, 1.45 mM KH₂PO₄, 137 mM NaCl, 2.7 mM KCL, pH 7.4). To covalently cross-link protein-RNA complexes in vivo, ice-cold PBS (5 ml) was added to cells, lid was removed and cells were exposed to 400 mJ/cm² irradiation in a wavelength of 254 nm. After adding 5 ml of ice-cold PBS, cross-linked cells were scraped and collected into 16 ml tubes. Cells were pelleted by 5 min centrifugation (1,000×G) in 4° C. Supernatant was removed and cell pellets were shock-frozen in liquid nitrogen and stored in −80° C. Protein G Dynabeads (Life Technologies) were utilized for pre-clearing and immunoprecipitation. Beads were thoroughly resuspended, and a volume of beads corresponding to 20 μl beads×number of samples+5 μl was transferred into a clean 1.5 ml tube. Beads were then captured on magnetic separator. Pre-clearing beads were washed 3 times with 1 ml lysis buffer (PBS supplemented with 1 mM MgCl₂, 0.1 mM CaCl₂, 0.5% Nonidet-P-40, and 0.5% Sodium Deoxycholate). Beads were resuspended in 100 μl lysis buffer per 20 μl beads and 100 μl portions transferred into 1.5 ml tubes. Beads for immunoprecipitation were washed 3 times with 1 ml lysis buffer (PBS supplemented with 1 mM MgCl₂, 0.1 mM CaCl₂, 0.5% Nonidet-P-40, and 0.5% Sodium Deoxycholate)+0.5% BSA. Beads were resuspended in 100 μl lysis buffer per 20 μl beads and 100 μl portions transferred into 1.5 ml tubes. 400 μl lysis buffer+0.5% BSA+5 μg of specific antibody were added and beads incubated 4 hrs in 4° C. on a rotatory wheel. To prepare cell lysate, cell pellets (1 pellet for each cell type) were resuspended in 1.25 ml of ice-cold lysis buffer supplemented with 1 tablet of Complete-mini EDTA-free tablet (Roche), 40 u/ml protector RNAse inhibitor (Roche), 1 mM Dithiothreitol (DTT), and transferred into 2 ml tube followed by 25 min incubation in 4° C. on rotatory wheel. After a brief spin down, 25 μl (50 U) of TurboDNAse (Life Technologies) were added to each tube. The entire content of each tube was then split equally between four 1.5 ml tubes. Two dilutions of RNAse I (Life Technologies) in lysis buffer containing additives were prepared: 10-fold (10 u/ml) and 100-fold (1 u/ml). Per each of the cell lines, three samples were prepared in growing concentrations of RNAse I: (1) undiluted RNAse I (×1) (2) 10-fold diluted, and (3) 100-fold diluted. Volume of RNAse I solution corresponded to 1/100th of total sample volume. The final dilutions of RNAse I were correspondingly 100-fold, 1,000-fold and 10,000-fold. In parallel, a fourth sample, untreated with RNAse I, was prepared and used as immunoprecipitation control for Western Blotting. Samples were thoroughly mixed, incubated for 15 min in a 37° C. water bath, and were gently mixed every 5 min. After a brief spin-down, each sample received 6 μl (12 U) of SuperRNAseIN (Life Technologies) 10-fold diluted in lysis buffer. Sodium dodecyl sulfate (SDS) concentrations per each sample were further brought up to 0.1% following by addition of 1/100th volume of 10% SDS. After 10 min 21,130×G centrifugation in 4° C., supernatant was transferred into a clean 1.5 ml tube and sample was centrifuged for another 10 min 21,130×G in 4° C. to remove remaining cell debris. 1.5 ml tubes supplemented with 100 μl of pre-clearing beads were put on magnetic separator and lysis buffer was removed. The entire supernatant from the previous step was placed on the beads and samples were further incubated 1 hr in 4° C. on a rotatory wheel. After capturing pre-clearing beads on magnetic separator, pre-cleared lysate samples were transferred into 1.5 ml tubes with protein G-antibody complex and incubated for 16 hrs in 4° C. on a rotatory wheel. Samples were placed on a magnetic separator and supernatant was removed. Samples were washed twice with 1 ml high-salt buffer (PBS supplemented with 750 mM NaCl, 1% Nonidet-P-40, 0.5% NaDeoxycholate, and 0.1% SDS) and three times with 1 ml low-salt buffer (PBS supplemented with 150 mM NaCl, 1% Nonidet-P-40, 0.5% NaDeoxycholate, and 0.1% SDS) for 5 min at 4° C. on a rotatory wheel per every wash, following by supernatant removal on a magnetic separator. IP control samples received 40 μl of NuPage 3× LDS sample buffer (Life Technologies), or alternatively, 40 μl SDS sample buffer ×1 (80 mM Tris pH 6.8, 2% SDS, 100 mM DTT, and 10% Glycerol). In the remaining RNAse-treated samples, beads were resuspended in 400 μl 1× DNAse buffer (Life Technologies) and incubated for 5 min at 4° C. on a rotatory wheel followed by subsequent supernatant removal on a magnetic separator. Beads were resuspended in 40 μl of DNAse mix (1× DNAse buffer, 0.1 u/μl Turbo DNAse, 0.1 u/μl SUPERasin (Life Technologies), 100-fold diluted EDTA-free protease inhibitors mix (Sigma), 0.4 u/μl Protector RNAse inhibitor) and incubated at 37° C. for 30 min. After a brief spin down, beads were placed on a magnetic rack and supernatant was removed. One wash with 0.5 ml low-salt washing buffer was performed for 5 min at 4° C. placed on a rotatory wheel. Supernatant was removed on a magnetic separator. For phosphorylation of 5′ ends, supernatant was removed on a magnetic separator and beads were washed once in 1 ml of PNK buffer (50 mM Tris pH 7.4, 10 mM MgCl₂, 5 mM DTT, 0.5% Nonidet-P-40) for 5 min at 4° C. placed on a rotatory wheel. Beads were then resuspended in 20 μl of PNK mix (per sample, 20 μl PNK buffer, 1 μl ³²P-gamma-ATP, 0.5 μl (5 U) T4 Polynucleotide Kinase (PNK) (NEB)) and incubated at 37° C. for 20 min. Beads were captured and supernatant was removed on a magnetic separator. Beads were instantly washed 3 times with 0.5 ml of ice-cold PNK washing buffer (50 mM Tris pH 7.4, 150 mM NaCl, 0.5% Nonidet-P-40, 10 mM EDTA). After the last wash, beads were resuspended in 40 μl of NuPage 3× LDS sample buffer (Life Technologies), or alternatively, in 40 μl of SDS sample buffer ×1 (80 mM Tris pH 6.8, 2% SDS, 100 mM DTT, 10% Glycerol). 3× LDS samples were incubated at 80° C. for 10 min. Samples with 1× SDS sample buffer were incubated at 95° C. for 5 min. Samples were then loaded on 1 mm NuPage 4%-12% Bis-Tris gradient gel (Life Technologies), and electrophoresis on 200V was performed using NuPage MOPS/SDS running buffer (50 mM Tris base, 50 mM MOPS, 0.1% SDS, 1 mM EDTA, pH 7.7). Transfer into nitrocellulose membranes was performed for 1 hr under 140 mA at 4° C., soaking in NuPage 1× transfer buffer (25 mM Bicine, 25 mM Bis-Tris (free base) 1 mM EDTA pH 7.2) supplemented with 12% methanol. Membrane was wrapped in saran plastic wrap and exposed to phosphoimager screen.

Denaturing CLIP Method—Small Scale

To perform the denaturing CLIP method, we raised by DNA vectors transfection, following by antibiotic selection, several cell lines expressing (1) a bacterial biotin ligase BirA vector carrying a neomycin-resistance marker, along with (2) a puromycin resistance expression vector of mouse CBX7 fused to a biotinylation tag. An empty biotinylation vector was alternatively transfected for generating control cell lines. Cells were grown to full confluence in 15 cm tissue culture dishes. Medium was then aspirated and cells were washed with 10 ml ice-cold phosphate-buffered saline (PBS) (containing 8.1 mM Na₂HPO₄, 1.45 mM KH₂PO₄, 137 mM NaCl, 2.7 mM KCL, pH 7.4). To covalently cross-link protein-RNA complexes in vivo, ice-cold PBS (5 ml) was added to cells, lid was removed and cells were exposed to 400 mJ/cm² irradiation in a wavelength of 254 nm. Day 7 differentiated cells grown in monolayer as well as HEK293 cells were exposed to 150 mJ/cm² irradiation in a wavelength of 254 nm. After adding 5 ml of ice-cold PBS, cross-linked cells were scraped and collected into 16 ml tubes. Cells were pelleted by 5 min centrifugation (1,000×G) in 4° C. Supernatant was removed and cell pellets were shock-frozen in liquid nitrogen and stored in −80° C. For performing protein pull-down, two types of magnetic beads were employed: (1) Protein G Dynabeads (for pre-clearing), and (2) Dynabeads® MyOne™ Streptavidin C1 (for biotinylated protein pull-down)—both bead types from Life Technologies. Beads were thoroughly resuspended, and a volume of beads corresponding to 20 μl beads×number of samples+5 μl was transferred into a clean 1.5 ml tube. Beads were then captured on magnetic separator. Pre-clearing beads were washed 3 times with 1 ml lysis buffer (PBS supplemented with 1 mM MgCl₂, 0.1 mM CaCl₂, 0.5% Nonidet-P-40, and 0.5% Sodium Deoxycholate). Streptavidin beads were washed 3 times with 1 ml lysis buffer containing 0.5% Bovine serum albumin (BSA). Beads were resuspended in 100 μl lysis buffer per 20 μl beads and 100 μl portions transferred into 1.5 ml tubes. Cell lysate was prepared the following manner: Cell pellets (1 pellet for each cell type) were resuspended in 1.25 ml of ice-cold lysis buffer (supplemented with 1 tablet of Complete-mini EDTA-free tablet (Roche), 40 u/ml protector RNAse inhibitor (Roche), 1 mM Dithiothreitol (DTT)), and transferred into 2 ml tube following by 25 min incubation in 4° C. on rotatory wheel. After a brief spin down, 25 μl (50 U) of TurboDNAse (Life Technologies) were added to each tube. The entire content of each tube was then split equally between four 1.5 ml tubes. Two dilutions of RNAse I (Life Technologies) in lysis buffer containing additives were prepared: 10-fold (10 u/ml) and 100-fold (1 u/ml). Per each of the cell lines, three samples were prepared in growing concentrations of RNAse I: (1) undiluted RNAse I (×1) (2) 10-fold diluted, and (3) 100-fold diluted. Volume of RNAse I solution corresponded to 1/100th of total sample volume. The final dilutions of RNAse I were correspondingly: 100-fold, 1,000-fold and 10,000-fold. In parallel, a fourth sample, untreated with RNAse I, was prepared and used as a pull-down control for Western Blotting. Samples were thoroughly mixed, incubated for 15 min in a 37° C. water bath, and were gently mixed every 5 min. After a brief spin-down, each sample received 6 μl (12 U) of SuperRNAseIN (Life Technologies) 10-fold diluted in lysis buffer. Sodium dodecyl sulfate (SDS) concentrations per each sample were further brought up to 0.1% following by addition of 1/100th volume of 10% SDS. After 10 min 21,130×G centrifugation in 4° C., supernatant was transferred into a clean 1.5 ml tube and samples were centrifuged for another 10 min 21,130×G in 4° C. to remove remaining cell debris. 1.5 ml tubes supplemented with 100 μl of pre-clearing beads were put on magnetic separator and lysis buffer was removed. The entire supernatant from the previous step was placed on the beads and samples were further incubated for 1 hr in 4° C. on a rotatory wheel. 1.5 ml tubes containing streptavidin beads were placed on a magnetic separator and any excess of lysis buffer was removed. After capturing pre-clearing beads on magnetic separator, pre-cleared lysate samples were transferred into 1.5 ml tubes supplemented with streptavidin beads and incubated for 2 hrs in 4° C. on a rotatory wheel. Samples were placed on a magnetic separator and supernatant was removed. Samples were washed twice with 0.5 ml wash buffer 1 (PBS containing 8M Urea and 0.1% SDS) for 5 min at room temperature swirling on rotatory wheel. Supernatant was removed by employing magnetic separator each time. Samples were washed twice with 0.5 ml Urea wash buffer (PBS+8M urea+0.1% SDS) and twice with 0.5 ml SDS wash buffer (PBS+2% SDS) for 5 min at room temperature and were swirled on a rotatory wheel. Supernatant was removed on magnetic separator per each cycle. One wash was performed with 0.5 ml high-salt buffer (PBS supplemented with 750 mM NaCl, 1% Nonidet-P-40, 0.5% NaDeoxycholate, and 0.1% SDS) and one time with 0.5 ml low-salt buffer (PBS supplemented with 150 mM NaCl, 1% Nonidet-P-40, 0.5% NaDeoxycholate, and 0.1% SDS) for 5 min at 4° C. on a rotatory wheel per every wash, following by supernatant removal on a magnetic separator. IP control samples received 40 μl of NuPage 3× LDS sample buffer (Life Technologies), or alternatively, 40 μl SDS sample buffer ×1 (80 mM Tris pH 6.8, 2% SDS, 100 mM DTT, and 10% Glycerol). In the remaining RNAse-treated samples, beads were resuspended in 400 μl 1× DNAse buffer (Life Technologies) and incubated for 5 min at 4° C. on a rotatory wheel followed by subsequent supernatant removal on a magnetic separator. Beads were resuspended in 40 μl of DNAse mix (1× DNAse buffer, 0.1 u/μl Turbo DNAse, 0.1 u/μl SUPERasin (Life Technologies), 100-fold diluted EDTA-free protease inhibitors mix (Sigma), 0.4 u/μl Protector RNAse inhibitor) and incubated at 37° C. for 30 min. After a brief spin down, beads were placed on a magnetic rack and supernatant was removed. One wash with 0.5 ml low-salt washing buffer was performed for 5 min at 4° C. placed on a rotatory wheel. Supernatant was removed on a magnetic separator. For phosphorylation of 5′ ends, supernatant was removed on a magnetic separator and beads were washed once in 0.5 ml of PNK buffer (50 mM Tris pH 7.4, 10 mM MgCl₂, 5 mM DTT, 0.5% Nonidet-P-40) for 5 min at 4° C. placed on a rotatory wheel. Beads were then resuspended in 20 μl of PNK mix (per sample, 20 μl PNK buffer, 1 μl ³²P-gamma-ATP, 0.5 μl (5 U) T4 Polynucleotide Kinase (PNK) (NEB)) and incubated at 37° C. for 20 min. Beads were captured and supernatant was removed on a magnetic separator. Beads were instantly washed 3 times with 0.5 ml of ice-cold PNK washing buffer (50 mM Tris pH 7.4, 150 mM NaCl, 0.5% Nonidet-P-40, 10 mM EDTA). After the last wash, beads were resuspended in 40 μl of NuPage 3× LDS sample buffer (Life Technologies), or alternatively, in 40 μl of SDS sample buffer ×1 (80 mM Tris pH 6.8, 2% SDS, 100 mM DTT, 10% Glycerol). 3× LDS samples were incubated at 80° C. for 10 min. Samples in 1× SDS sample were incubated at 95° C. for 5 min. Samples were then loaded on lmm NuPage 4%-12% Bis-Tris gradient gel (Life Technologies), and electrophoresis on 200V was performed using NuPage MOPS/SDS running buffer (50 mM Tris base, 50 mM MOPS, 0.1% SDS, 1 mM EDTA, pH 7.7). Transfer into nitrocellulose membranes was performed for 1 hr under 140 mA at 4° C., soaking in NuPage 1× transfer buffer (25 mM Bicine, 25 mM Bis-Tris (free base) 1 mM EDTA pH 7.2) supplemented with 12% methanol. Membrane was wrapped in saran plastic wrap and exposed to phosphoimager screen.

Denaturing CLIP Method—Large Scale for Library Preparation.

For large-scale denaturing CLIP method, UV treatment of cells was performed as described above for small scale dCLIP method. Two kinds of magnetic beads were used for the experiment: Protein G Dynabeads (for Pre-clearing) and Dynabeads® MyOne™ Streptavidin C1 (for a Pull-down of biotinylated protein). Beads were thoroughly resuspended and volume of beads corresponding to 80 μl beads×number of cell pellets+5 μl was transferred into clean 2 ml tubes. Beads were captured on magnetic separator. Pre-clearing beads were washed 3 times with 1 ml lysis buffer (PBS+1 mM MgCl₂+0.1 mM CaCl₂+0.5% Nonidet-P-40+0.5% Sodium Deoxycholate). Streptavidin beads were washed 3 times with 1 ml lysis buffer+0.5% Bovine serum albumin (BSA) Beads were resuspended in 150 μl lysis buffer per 80 μl beads and 150 μl portions transferred into 2 ml tubes. Lysate was prepared the following way. Cell pellets (2 pellets for each cell type) were resuspended each in 1.25 ml of ice-cold lysis buffer supplemented with 1 tablet of Complete-mini EDTA-free tablet (Roche)+40 u/ml Protector RNAse inhibitor (Roche)+1 mM Dithiothreitol (DTT), delivered into 2 ml tubes and incubated for 25 min at 4° C. on rotatory wheel. After brief spin down, 25 μl (50 u) of TurboDNAse (Life Technologies) were added to every tube. The entire content of the tube was transferred to the new 2 ml tube in order to estimate the volume. 2 dilutions of RNAse I (Life Technologies) in lysis buffer+additives were prepared: 100-fold (1 u/ml) and 500-fold (0.2 u/ml) For each cell type, one sample received 100-fold and one sample received 500-fold diluted RNAse I. Volume of RNAse I solution corresponded to 1/100th of total estimated sample. Samples were mixed well and incubated 15 min in 37° C. water bath with mixing up-and-down every 5 min. After brief spin-down, each sample received 24 μl (48 u) of SuperRNAseIN (Life Technologies) diluted 10-fold in lysis buffer. In addition, the sodium dodecyl sulfate (SDS) concentration in each sample was brought up to 0.1% following addition of + 1/100th volume of 10% SDS. After 10 min 21,130×G 4° C. centrifugation, sup was delivered into clean 2 ml tubes and samples centrifuged another 10 min 21,130×G 4° C. to remove remaining cell debris. 2 ml tubes with 150 μl of pre-clearing beads were placed on magnetic separator and lysis buffer removed. The entire sup from the previous step was placed on the beads and samples incubated 1 hr 4° C. on rotatory wheel. 2 ml tubes with Streptavidin beads were placed on magnetic separator and excess lysis buffer removed. After capturing pre-clearing beads on magnetic separator, pre-cleared lysate was transferred into 2 ml tubes with Streptavidin beads and incubated 2 hrs 4° C. on rotatory wheel. Samples were placed on magnetic separator and sup removed. Samples were washed 2 times with 1.2 ml Urea wash buffer (PBS+8M Urea+0.1% SDS) for 5 min on room temperature using rotatory wheel. Sup was removed on magnetic separator every time. Samples were washed 2 times with 1.2 ml SDS wash buffer (PBS+2% SDS) for 5 min on room temperature using rotatory wheel. Sup was removed on magnetic separator every time. One wash was performed with 1.2 ml high-salt buffer (PBS+750 mM NaCl+1% Nonidet-P-40+0.5% NaDeoxycholate+0.1% SDS) and one wash with low-salt buffer (PBS+150 mM NaCl+1% Nonidet-P-40+0.5% NaDeoxycholate+0.1% SDS), 5 min 4° C. on rotatory wheel for every wash with subsequent sup removal on magnetic separator. Beads were resuspended in 800 μl 1× DNAse buffer, transferred into 1.5 ml tubes and incubated for 5 min 4° C. on rotatory wheel with subsequent sup removal on magnetic separator. Beads were resuspended in 160 μl of DNAse mix (1× DNAse buffer, 0.1 u/μl Turbo DNAse, 0.1 u/μl SUPERasin (Life Technologies), 100-fold diluted EDTA-free protease inhibitors mix (Sigma), 0.4 u/μl Protector RNAse inhibitor) and incubated at 37° C. on rotatory wheel for 30 min. After brief spin down, beads were placed on magnetic rack and sup removed. One wash with 1 ml low-salt wash buffer was performed 5 min 4° C. on rotatory wheel. Sup removed on magnetic separator. For 3′ends dephosphorylation, beads were washed once in 1 ml of Low_pH_PNK buffer (70 mM Tris pH 6.5, 10 mM MgCl₂, 5 mM DTT) 5 min 4° C. on rotatory wheel. Low-pH-PNK mix (per sample, 80 μl Low_pH_PNK buffer, 2 μl (20 u) T4 polynucleotide kinase (T4 PNK) (NEB), 2 μl (80 u) Protector RNAse inhibitor) was prepared. Beads resuspended in 80 μl of Low-pH-PNK mix and incubated at 37° C. for 20 min on Thermomixer, vortexing on 1,000RPM for 15 sec every 2 min. For subsequent phosphorylation of 5′ ends, sup was removed on magnetic separator and beads washed once in 1 ml of PNK buffer (50 mM Tris pH 7.4, 10 mM MgCl₂, 5 mM DTT, 0.5% Nonidet-P-40) 5 min 4° C. on rotatory wheel. Beads were resuspended in 80 μl of PNK mix (per sample, 80 μl PNK buffer, 4 μl ³²P-gamma-ATP, 3 μl (30 u) T4 PNK, 2 μl (80 u) Protector RNAse inhibitor) and incubated at 37° C. for 10 min. After adding 8 μl 10 mM ATP, samples were incubated additional 20 min at 37° C. Beads were captured and sup removed on magnetic separator. Beads were instantly washed 3 times with 1 ml of ice-cold PNK wash buffer (50 mM Tris pH 7.4, 150 mM NaCl, 0.5% Nonidet-P-40, 10 mM EDTA). After last wash, beads were resuspended in 85 μl of 1× SDS sample buffer (80 mM Tris pH 6.8, 2% SDS, 100 mM DTT, 10% Glycerol) and incubated at 95° C. for 5 min. Samples were loaded on 1.5 mm NuPage 4%-12% Bis-Tris gradient gels (Life Technologies)—40 μl per lane, and electrophoresis on 200V was performed using NuPage MOPS/SDS running buffer (50 mM Tris base, 50 mM MOPS, 0.1% SDS, 1 mM EDTA, pH 7.7). Transfer into nitrocellulose membranes was performed for 1.5 hrs 140 mA on 4° C. in NuPage 1× transfer buffer (25 mM Bicine, 25 mM Bis-Tris (free base) 1 mM EDTA pH 7.2)+12% methanol. Membrane was wrapped in Saran plastic wrap and briefly exposed to phosphoimager screen.

Samples that were subjected for beads elution protocol, were treated with PNK mix supplemented with 8 μl 10 mM ATP and incubated at 37° C. for 30 min. Samples were washed twice with 1 ml Urea wash buffer (PBS+8M Urea+0.1% SDS) and once with Proteinase K buffer (100 mM Tris pH 8.0, 200 mM NaCl, 5 mM EDTA, 0.1% SDS) for 5 min on room temperature using rotatory wheel. Beads were resuspended in 200 μl Proteinase K mix (100 mM Tris pH 8.0, 200 mM NaCl, 5 mM EDTA, 0.1% SDS, lmg/ml Proteinase K (PCR grade, Roche, 20 mg/ml)) and incubated 30 min at 55° C. using rotatory wheel. After brief spin-down and beads capture on magnetic separator, eluted RNA from 2 combined samples (400 μl total) was transferred into phase-lock gel 2 ml tubes (5 Prime) (pre-centrifuged 30 sec 16,000×G to pellet gel), and 400 μl acidic phenol-chloroform (Life Technologies) were added. After rigorous up-and-down shaking to mix the phases, samples were centrifuged 5 min 16,000×G on room temperature. Another 400 μl of acidic phenol-chloroform were added to the upper aqueous phase with subsequent rigorous up-and-down shaking to mix the phases and 5 min 16,000×G centrifugation on room temperature. Upper phase was transferred into clean 1.5 ml tubes with 40 μl 3M Sodium Acetate. After addition of 1 μl Glycoblue (Life Technologies) and 1 ml 100% ethanol, samples were mixed by up-and-down shaking and incubated at least 16 hrs on −20° C.

Elution of Protein-Bound RNAs from Membrane

For membrane elution, PK solution (100 mM Tris pH 7.4, 50 mM NaCl, 10 mM EDTA, 4 mg/ml Proteinase K (Roche)) was prepared and pre-incubated for 10-20 min at room temperature to eliminate possible RNAse contamination. All the solutions were filtered through 0.22 μm membrane filter before adding Proteinase K. Membrane pieces were excised using a sterile scalpel starting from protein of interest size+10 kda (corresponding to approximately 30 bases of RNA fragments covalently linked to the protein of interest) up to the end of visible radioactive signal specific to the protein of interest. Addition of 10 kDa to the original protein size allows for binding of roughly 30 bases of RNA in complex with the protein of interest. The goal was to avoid purifying CBX7 crosslinked to RNAs shorter than 30 bases, as the shorter RNAs would be more difficult to sequence and align to the genome with high confidence level. Membrane pieces were further cut into smaller pieces and placed into low-binding 1.5 ml tubes. After addition of 200 μl PK buffer, membrane pieces were incubated for 20 min at 55° C. in Thermomixer with constant vortexing on 1,200 RPM. Meanwhile, PK-urea solution was prepared (100 mM Tris pH 7.4, 50 mM NaCl, 10 mM EDTA, 7M urea). All the solutions except for urea were filtered through 0.22 μM membrane filter before preparation. 200 μl of PK-urea solution was further added to membrane pieces. Samples were incubated for 20 min at 55° C. in Thermomixer with constant vortexing on 1,200 RPM. After brief spin-down, the eluates were transferred into phase-lock gel 2 ml tubes (5 Prime) (pre-centrifuged 30 sec 16,000×G to pellet gel), and 400 μl acidic phenol-chloroform (Life Technologies) were added. After rigorous up-and-down shaking to mix the phases, samples were centrifuged 5 min 16,000×G on room temperature. Another 400 μl of acidic phenol-chloroform were added to the upper aqueous phase with subsequent rigorous up-and-down shaking to mix the phases and 5 min 16,000×G centrifugation on room temperature. Upper phase was transferred into clean 1.5 ml tubes with 40 μl 3M Sodium Acetate. After addition of 1 μl Glycoblue (Life Technologies) and lml 100% ethanol, samples were mixed by up-and-down shaking and incubated at least 16 hrs on −20° C.

Library Preparation from Membrane-Eluted and Beads-Eluted Samples

Membrane-eluted or beads-eluted samples were centrifuged for 30 min 13,523×G on 4° C. and sup removed. Pellets were washed once with 1 ml 75% ethanol in DEPC-treated water with subsequent 10 min 13,523×G centrifugation on 4° C. After sup removal and short spin down, the remaining ethanol solution was carefully removed and pellets incubated 5-10 min with open cup on room temperature inside a PCR workstation under constant airflow. Pellets were eluted in 25 μl DNAse mix and 2 samples that belonged to the same cell type with different RNAse concentrations combined into one sample (DNAse mix: per combined sample, 43 μl nuclease-free DDW, 5 μl 10× DNAse buffer, 1 μl (40 u) Protector RNAse inhibitor, 1 μl (2 u) of Turbo-DNAse (Life Technologies). Samples were incubated 30 min on 37° C. RNA was extracted using 950 μl Trizol reagent (Life Technologies) according to the manufacturer instructions. 0.5 μl Glycoblue were used for precipitation during Trizol extraction. RNA pellets were eluted with 8 μl nuclease-free DDW. Samples were incubated for 2 min on 70° C. to reduce the secondary structure and then kept on ice. Multiplex Compatible NEBNext Small RNA Library Prep Set for Illumina (NEB) was used for library preparation according to manufacturer instructions with the following modifications. 7 μl of eluted RNA were used for library preparation. All the adapters and primers used throughout a procedure were diluted 12-fold. SuperScript III Reverse Transcriptase (Life Technologies) and Protector RNAse inhibitor (Roche) replaced M-MuLV reverse transcriptase and Murine RNAse inhibitor respectively. 25 PCR amplification cycles were performed on the resulting cDNA using multiplexed primers with Illumina barcodes—distinct barcode for every cell type. Amplification was performed with LongAmp™ Taq 2× Master Mix (NEB). Amplified PCR products were subjected to PAGE electrophoresis on 6% TBE-acrylamide gel. The area between 160 bp and 520 bp was excised, gel pieces crushed into slurry with 1 ml syringe plunger and PCR products eluted by overnight incubation on room temperature in 400 μl Gel elution buffer (NEB) inside a 1.5 ml low-binding tubes. One glass filter (Whatman, 1823010) was placed into Costar SpinX column (Cornig, 8161). The suspension from the previous step was placed on the column and centrifuged on 15,871×G on room temperature for 1 min. Eluates were subjected to ethanol precipitation following addition of 40 μl 3M Sodium Acetate, 1 μl Glycoblue and lml 100% ethanol. After incubating for at least 30 min on −20° C., samples were centrifuged for 30 min 13,523×G on 4° C. and sup removed. Pellets were washed once with 1 ml 70% ethanol with subsequent 10 min 13,523×G centrifugation on 4° C. After sup removal and short spin down, the remaining ethanol solution was carefully removed and pellets incubated 5-10 min with open cap on room temperature inside a PCR workstation under constant airflow. Pellets were eluted in 12 μl nuclease-free water. Size distribution of PCR products was determined by Bioanalyzer run with 1 μl of each sample loaded on High Sensitivity DNA chip (Agillent). Quantification of PCR products was performed by Illumina Library Quantification kit (Kapa Biosystems). Equivalent amounts of multiplexed samples were pooled into final library—1.5 nM-2 nM per multiplexed sample. Total of 3 to 5 samples were pooled into one library.

Gene Expression

RNA was extracted from cells with Trizol reagent (Life Technologies) according to manufacturer instructions. cDNA libraries were constructed using Superscript III reverse-transcriptase (Life Technologies) and qPCR was performed with primers spanning exon-exon junctions. For studies involving intronic primers (FIG. 6D,E), contaminating DNA was removed from RNA prior to reverse transcription by Turbo DNA-free kit (Life Technologies). Primer sequences are given in Table a.

TABLE a
LNA ASO oligomers, primers, and RNA-EMSA probes used in this study.
LNA Oligomers

| Target Gene | LNA I.D | LNA sequence | SEQ ID NO. |
|---|---|---|---|
| Dusp9 | Dusp9-1-a | CCTACAGTTCCAAGAAGTCTAA | 36405 |
| | Dusp9-1-b | GAAGCAGGAAGGAGTCTACACG | 36406 |
| | Dusp9-2-a | CAGTTTGACCACCCTCAGTCAC | 36407 |
| | Dusp9-2-b | AAAGAAACAGTCAGGGCACCAG | 36408 |
| | Dusp9-3-a | CACAGGTATTGCCAGCTCCAGG | 36409 |
| | Dusp9-3-b | CACACACACAGAGTCTACAACG | 36410 |
| Dcaf1211 | Dcaf12I1-1 | CCTGTCTGCCATACATTCTACA | 36411 |
| | Dcaf12I1-2 | GCTCAGACTTCTTCCTTTGCAC | 36412 |
| | Dcaf12I1-3 | GTAACAGATCTATTCTACTTGA | 36413 |
| | Dcaf12I1-4-a | CATTATCTCTATTTATCTGAAC | 36414 |
| | Dcaf12I1-4-b | GGAGAAAACCAATCTATCCGCA | 36415 |
| Calm2 | Calm2-1-a | GCCAGAGTAAGCCACATGCAAC | 36416 |
| | Calm2-1-b | TTAGATGTGCAGACGGGCTTAG | 36417 |
| | Calm2-2-a | TTACAGCTCCACACTTCAACAAC | 36418 |
| | Calm2-2-b | ACATGCTGACAGTTCCTAAAAG | 36419 |
| Control LNAs | LNA-Scr | GTGTAACACGTCTATACGCCCA | 36420 |
| | Negative control | TAACACGTCTATACGCCCA | 36421 |
| | A | | |

Native RIP-qPCR primers

| | | |
|---|---|---|
| Tug1_F | CAG GTC TGT AGG CTG ATG GAG | SEQ ID NO. 36422 |
| Tug1_R | AAG TGA ACT ACG TCC CGT GC | SEQ ID NO. 36423 |
| Dusp9_4F | TCA CAC AGC CAC TGT TGG TT | SEQ ID NO. 36424 |
| Dusp9_4R | GTC CTG CTG CCA CAG GTA TT | SEQ ID NO. 36425 |
| Calm2_F | GCA GAA CTG CAG GAC ATG AT | SEQ ID NO. 36426 |
| Calm2_R | CAA ACA CAC GGA ATG CTT CT | SEQ ID NO. 36427 |
| U1-F | GGAAATCATACTTACCTGGC | SEQ ID NO. 36428 |
| U1-R | AAACGCAGTCCCCCACTACC | SEQ ID NO. 36429 |

Gene Expression primers

| | | |
|---|---|---|
| Dusp9_F | GGG GAT CCG TCT CCA TGA AC | SEQ ID NO. 36430 |
| Dusp9_ChIP_R2 | TGA CCG ACT CAG ACT CTC CA | SEQ ID NO. 36431 |
| Calm2_F | GCA GAA CTG CAG GAC ATG AT | SEQ ID NO. 36432 |
| Calm2_R | CAA ACA CAC GGA ATG CTT CT | SEQ ID NO. 36433 |
| Dcaf12I1-1F | CCC AAT GCG CTC TAC ACT CA | SEQ ID NO. 36434 |
| Dcaf12I1-1R | ACT GGA TAC TCT GGG GCA GT | SEQ ID NO. 36435 |

Intronic primers

| | | |
|---|---|---|
| Calm2_int_F | GCC AAG CAA ACT TGA CTC CG | SEQ ID NO. 36436 |
| Calm2_int_R | GAC CAC ACT GCC ATG GAT CA | SEQ ID NO. 36437 |
| Dcaf12I1-1R | ACT GGA TAC TCT GGG GCA GT | SEQ ID NO. 36438 |
| Dcaf12I1-int-R | TGT AAT TCA TGT TGT GCA TGC TGT | SEQ ID NO. 36439 |

ChIP-qPCR primers

| | | |
|---|---|---|
| Calm2_ChIP_1F | AGC TAT ATG CAC CCA CTC GG | SEQ ID NO. 36440 |
| Calm2_ChIP_1R | TGG GCA TTC GTT CGA AAG GG | SEQ ID NO. 36441 |
| Dcafl2I1_ChIP F | CCA GAG TGG GCA ACT GGT AG | SEQ ID NO. 36442 |
| Dcaf12I1_ChIP R | GAC CAC ATC ATG CGC ATT CC | SEQ ID NO. 36443 |

FAIRE-qPCR primers

| | | |
|---|---|---|
| Calm2_ChIP_1F | AGC TAT ATG CAC CCA CTC GG | SEQ ID NO. 36444 |
| Calm2_ChIP_1R | TGG GCA TTC GTT CGA AAG GG | SEQ ID NO. 36445 |
| Calm2_DNAse_1A_F | GGG GAC GGA TGA CGT AAG TG | SEQ ID NO. 36446 |
| Calm2_DNAse_1A_R | AAT CAG CAG CAA GCT CAA CG | SEQ ID NO. 36447 |
| Dcaf12I1_ChIP_F | CCA GAG TGG GCA ACT GGT AG | SEQ ID NO. 36448 |
| Dcaf12I1_ChIP_R | GAC CAC ATC ATG CGC ATT CC | SEQ ID NO. 36449 |
| Dcaf12I1_DNAse_72F | GTC GGC CTG ACG CAT GAT A | SEQ ID NO. 36450 |
| Dcaf12I1_DNAse_72R | GCT GAT CGG TTG ATC GCT CT | SEQ ID NO. 36451 |

qPCR primers-human genes

| | | |
|---|---|---|
| PES1-F | GAG GAG AAG TGA CTC TGG TCC AT | SEQ ID NO. 36452 |
| PES1-R | AGA AGC GGA AAG CCC ACG AT | SEQ ID NO. 36453 |
| IRAK1-F | CAC ATT AGG CCA GCT CGC AG | SEQ ID NO. 36454 |
| IRAK1-R | TGG CTG TAA GTC TCA TGG TTC A | SEQ ID NO. 36455 |

RNA-EMSA Probes

| | | |
|---|---|---|
| Dusp9-EMSA probe | GGCCACTTTGACTCGTGTAGACTCCTTCCT | SEQ ID NO. 36456 |
| | GCTTCTCTCACTAGGG | |
| | CTTAGACTTCTTGGAACTGTAGGGTGTGA | |
| | ACCCAGAGAC | |
| Dcaf12I1-EMSA probe | TCAAATAGAGGAGCTGGGGATTAAAAAG | SEQ ID NO. 36457 |
| | ATAGGTCTGATTAAAG | |
| | GACTGTGCAGTTCAGATAAATAGAGATAA | |
| | TGGGATGCCGTGCGG | |
| | ATAGATTGGTTTTCTCC | |
| Calm2-EMSA probe | GTAGCTTTTAGGAACTGTCAGCATGTTGTT | SEQ ID NO. 36458 |
| | GTTGAAGTGTGGAGC | |
| | TGTAACTCTGCGTGGACTGTGGACAGTCA | |
| | ACAATATGTACTTAAAA | |
| | GTTGCACTATTGCAA | |
| Larp1-FAM1-WT | GGGAGGTATATGTGGACATAGAG | SEQ ID NO. 36459 |
| Larp1-FAM1-Mut | GGGAGGTATATTCCACCATAGAG | SEQ ID NO. 36460 |
| Nucks1-FAM3-WT | GGGtgtgcggacggaggtcagaaa | SEQ ID NO. 36461 |
| Nucks1-FAM3-Mut | GGGtgtgcggaccctcctcagaaa | SEQ ID NO. 36462 |

Native RNA Immunoprecipitation (Native-RIP)

EL16.7 cells were grown on T75 flask until ˜80% confluency. Cells were trypsinized and, after adding fresh growth media, counted and pelleted by 5 min 200×G centrifugation. Cell pellets were resuspended in PBS and divided into 1×10⁷ cells aliquots. After another round of centrifugation, sup was removed and cells shock-frozen in LN2. After thawing, cells from single cell pellet were incubated in 1 ml of ice-cold hypothonic buffer A (10 mM HEPES pH 7.9, 1.5 mM MgCl₂, 10 mM KCl)+1 mM AEBSF. Cells were incubated on ice for 20 min and nuclei were pelleted by 15 min 2,500×G centrifugation on 4° C. Sup was removed and pellet resuspended in 1 ml of Polysomal lysis buffer (10 mM HEPES pH 7.0, 100 mM KCL, 5 mM MgCl₂, 0.5% NP-40)+1 mM DTT+EDTA-free PI cocktail 1:100+100 u/ml RNAseIN (Promega). After adding 20 μl (40 u) of TurboDNAse (Life Technologies), cell nuclei were incubated 30 min 4° C. on orbital shaker. After 10 min 16,000×G 4° C. centrifugation, supernatant was transferred into 16 ml tube with 9 ml NT2 buffer (50 mM Tris pH 7.4, 150 mM NaCl, 1 mM MgCl₂, 0.05% NP-40)+10 μl 1M DTT+10 μl RNAseIN (Promega)+1 tablet of Complete-mini EDTA-free protease inhibitors mix. On the same time, Protein G Dynabeads were prepared—20 μl per sample for pre-clearing×number of samples+20 μl per sample for immunoprecipitation×number of samples. Beads were thoroughly resuspended, captured on magnetic separator and washed 3×1 ml NT2 buffer (50 mM Tris pH 7.4, 150 mM NaCl, 1 mM MgCl₂, 0.05% NP-40). After final resuspension—20 μl beads/100 μl NT2 volume, beads intended for immunoprecipitation were incubated with 5 μg of specific antibody (anti-CBX7 (P-15), Santa Cruz Biotechnologies) or Rabbit IgG control (Abcam) in total volume of 500 μl NT2 buffer. To prepare pre-cleared lysates, 1 ml aliquots of lysate were transferred into 1.5 ml tubes with 100 μl beads suspension and incubated 1 hr 4° C. on rotatory shaker. Input samples were prepared by taking 100 μl aliquots from lysate+900 μl Trizol reagent. After capturing beads on magnetic separator, pre-cleared lysates were transferred into 1.5 ml tubes with beads-antibody complex (unbound antibody fraction was removed on magnetic separator). After 3 hrs 4° C. incubation on rotatory shaker, sup was removed and beads washed 5×1 ml NT2 buffer. After the last wash solution was removed on magnetic separator, beads were resuspended in 1 ml Trizol reagent. RNA was extracted according to manufacturer protocol, eluted in 20 μl nuclease-free water and 2 μl of eluted RNA was subjected to reverse transcription using SuperScript III (Life technologies) according to manufacturer instructions. qPCR assays were performed on CFX96 real-time PCR system (Bio-Rad). Specific primers are listed in Table a. Threshold cycle values were translated into initial template amount for each sample based on the standard curve prepared from known quantities of EL16.7 cDNA. Enrichment of specific RNA species was expressed as percentage of total input RNA for each reaction.

Chromatin Immunoprecipitation

Before the experiment, cells were grown on 15 cm feeder plates up to 80-90% confluence. Medium was removed and cells washed once with 20 ml PBS. After 10 min incubation on 37° C. in 3 ml trypsin-EDTA (Gibco), cells were passed twice through 200 μl tip in 9 ml growth media using 13 ml pipette, transferred into 50 ml tubes with 18 ml growth media and counted. Cells were centrifuged 5 min 200×G on room temperature. Sup was removed and cells resuspended in 40 ml fresh growth media and split into 2×15 cm tissue culture dishes—20 ml per dish. Cells were incubated 45 min on 37° C. for feeder removal. Floating cells were collected into 50 ml tube and counted. Then, 1/10th volume of cross-linking solution (50 mM HEPES-KOH pH. 7.5, 100 mM NaCl, 1 mM EDTA, 0.5 mM EGTA, 11% Formaldehyde) were added and cells incubated 20 min room temperature on rotatory shaker followed by quenching with 1/20th volume of 2.5M Glycine solution. After 5 min 700×G centrifugation on 4° C., cells were washed twice with 30 ml of ice-cold PBS and pellet resuspended in volume of ice-cold PBS according to 3 ml PBS/5×10⁶ cells ratio. Cells were divided into 3 ml portions in 16 ml tubes and centrifuged 5 min 700×G 4° C. Sup was aspirated and pellets shock-frozen in liquid nitrogen and stored on −80° C. On the day of immunoprecipitation, cell pellets were pre-thawed on 4° C. and re-suspended in 1 ml Buffer#1 (50 mM HEPES-KOH pH 7.5, 140 mM NaCl, 1 mM EDTA, 10% Glycerol, 0.5% NP-40, 0.25% Triton-X-100) supplemented with protease inhibitors mix (Sigma). After 10 min 4° C. incubation on rotatory shaker, cells were spun 5 min 1400×G 4° C. Sup was aspirated and pellets resuspended in 1 ml Buffer#2 (10 mM Tris pH 8.0, 200 mM NaCl, 1 mM EDTA, 0.5 mM EGTA) supplemented with protease inhibitors mix (Sigma). After 10 min 4° C. incubation on rotatory shaker, cells were spun 5 min 1,400×G 4° C. Sup was aspirated and pellets resuspended in 1.2 ml of Buffer#3 (10 mM Tris pH 8.0, 1 mM EDTA, 0.5 mM EGTA) supplemented with protease inhibitors mix (Sigma). After 10 min 4° C. incubation on rotatory shaker, 70 μl 10% N-lauroyl-sarcosine were added, cell nuclei suspension transferred into screw-cap 15 mm×19 mm Covaris tubes and sonicated in Covaris E220 system with the following conditions: Duty—10%, Peak Incident Power—175, Cycles per burst—200, Duration—2400 sec (40 min). After sonication, cells were transferred into 1.5 ml tubes and centrifuged for 10 min 14,000×G on 4° C. Sup was transferred into 1.5 ml tubes, 20 μg of RNAse A (Roche) were added and samples incubated for 30 min on 37° C. After incubation, 55 μl aliquots were taken from each sample for input control and the rest divided into 2×550 μl portions in 1.5 ml tubes. Input samples were stored on −20° C. 275 μl of freshly prepared solution (3% Triton-X-100, 0.3% NaDeoxycholate, 3 mM EDTA)+protease inhibitors mix were added to 550 μl samples along with specific antibody or matched isotype controls and samples incubated 16 hrs 4° C. on rotatory shaker. For recombinant CBX7-Flag-HA, 5 μl of rabbit polyclonal anti-hemagglutinin tag antibody (H6908, Sigma) were used per reaction. For endogenous CBX7, 5 μg of rabbit polyclonal anti-CBX7 antibody (ab21873, Abcam) were used per reaction. 5 μg of Ubiquityl-Histone H2A (Lys119) (D27C4) #8240 antibody (Cell Signaling) were used for pull-down of ubiquitynated histone H2A. Meanwhile, magnetic protein G dynabeads (Life technologies)—40 μl per reaction, were washed twice with Buffer#1 using magnetic stand and blocked for 1 hr on 4° C. with 250 μg/ml salmon sperm DNA (Life technologies). After two washes with Buffer#1, beads were resuspended in buffer#1 according to 40 μl beads/100 μl buffer ratio and divided into 1.5 ml tubes. After removal of buffer, immunoprecipitated samples were transferred to 1.5 ml tubes with protein G dynabeads for additional 2-3 hrs 4° C. incubation on rotatory shaker. Then, sup was removed and beads washed 3×0.5 ml RIPA-1 buffer (50 mM HEPES-KOH, pH 7.5, 0.5 m LiCl, 0.7% NaDeoxycholate, 1% NP-40, 10 mM EDTA) and 3×0.5 ml RIPA-2 buffer (50 mM HEPES-KOH, pH 7.5, 0.25 m LiCl, 0.7% NaDeoxycholate, 1% NP-40, 10 mM EDTA). Beads were resuspended manually on each step. After one wash with 0.5 ml TEN buffer (10 mM Tris pH 8.0, 50 mM NaCl, 1 mM EDTA), beads were resuspended in 0.2 ml TES buffer (50 mM Tris pH8.0, 10 mM EDTA, 1% SDS) and incubated 15 min 65° C. with occasional vortexing and subsequent spin down. 145 μl of TES buffer were added to input samples. 40 μg of Proteinase K (Roche) were then added to all samples and samples incubated 16 hrs on 65° C. Then, after addition of 0.2 ml of TE buffer, the entire volume was transferred to Phase-Lock Gel Heavy 2 ml tubes (5 Prime GmbH) and extracted with 0.4 ml of phenol:chlorophorm:isoamyl alcohol solution (25:24:1, USB) according to manufacturer protocol. The aqueous phase was collected and ethanol precipitated by adding 40 μl of 3M NaAcetate, 25 μg GlycoBlue reagent (Life technologies) and 2.5 volumes of 100% ethanol. Elution was performed with 50 μl TE buffer pH 8.0. qPCR assays were performed on CFX96 real-time PCR system (Bio-Rad). Specific primers are listed in Table a. Threshold cycle values were translated into initial template amount for each sample based on the standard curve prepared from known quantities of genomic DNA. Enrichment of specific PCR amplicons was expressed as percentage of total input DNA for each reaction. ChIP-seq libraries were constructed using the NEBNext ChIP-Seq Library Prep Master Mix Set (NEB). Libraries were subjected to high-throughput sequencing using Illumina HiSeq 2000 apparatus according to manufacturer instructions. Approximately 40 million paired-end 50 bp reads were generated for every ChIP-seq sample.

LNA Nucleofection

LNA mixmers (Exiqon) were designed specifically against a CBX7 binding regions of selected genes (See Table a for the list of LNA oligomers) A total of 2×10⁶ EL16.7 cells, after feeder removal, were resuspended in 100 μL of ES cell nucleofector solution (Lonza). LNA oligos were added to a final concentration of 2 μM. The cells were transfected using the A-013 program on Amaxa Nucleofector II. 0.5 mL of culture medium were added and cell suspension was divided equally between two wells in gelatinized 6-well dish. For RT-qPCR, cells were harvested in 1 ml Trizol reagent 24 hrs after nucleofection, RNA extraction was performed according to manufacturer instruction. For Western Blotting, cells were scraped in 300 μl of SDS sample buffer (50 mM Tris pH 6.8, 100 mM DTT, 2% SDS, 0.1% bromophenol blue, 10% glycerol) and resulting extracts boiled on 95° C. for 5 min. For ChIP and Formaldehyde-assisted Isolation of Regulatory Elements (FAIRE) assays, three nucleofection reactions were pooled into one gelatinized 10 cm dish and cells harvested for cross-linking according to the ChIP or FAIRE protocol.

Formaldehyde-assisted Isolation of Regulatory Elements (FAIRE) analysis FAIRE analysis of nucleofected cells was performed as described in Simon et al (Simon et al., 2012) with following modifications. 24 hours after nucleofection, cells growing on gelatin-coated 10 cm tissue culture dishes were trypsinized with 1 ml Trypsin-EDTA solution. After most of the cells detached from the surface, 9 ml growth media were added to the plate and cells passed 2 times through 200 μl pipette tip. Then, 1/10^{th} volume of cross-linking solution (50 mM HEPES-KOH pH. 7.5, 100 mM NaCl, 1 mM EDTA, 0.5 mM EGTA, 11% Formaldehyde) were added and cells subjected to 5 min incubation on room temperature with constant rotation followed by 5 min quenching with 1/20^{th} volume of 2.5M Glycine solution. After 5 min 700×G centrifugations on 4° C., cells were washed 3 times with 10 ml of ice-cold PBS. Sup was aspirated and pellets shock-frozen in liquid nitrogen and stored on −80° C. On the day of the assay, cell pellets were pre-thawed on 4° C. and re-suspended in 1 ml lysis buffer Buffer A (10 mM HEPES-KOH pH 7.5, 100 mM NaCl, 1 mM EDTA, 1% SDS, 2% TX-100) After 10 min 4° C. incubation on rotatory shaker, cells were delivered into screw-capped 1.3 ml Covaris 15 mm×19 mm tubes and sonicated in Covaris E220 system in the following conditions: Duty—10%, Peak Incident Power—175, Cycles per burst—200, Duration—600 sec (10 min). After sonication, cells were transferred into 1.5 ml tubes and centrifuged for 5 min 20,000×G on 4° C. to pellet cell debris. Sup was transferred into 1.5 ml tubes. 100 μl aliquots from each sample were taken as input controls. Then, 2 aliquots of 300 μl from each lysate were transferred to Phase-Lock Gel Heavy 1.5 ml tubes (pre-centrifuged for 30 sec on 16,000×G), and extracted twice with 300 μl of phenol:chlorophorm:isoamyl alcohol solution (25:24:1, USB) after vigorous shaking and 5 min 16,000×G centrifugation on room temperature. The remaining phenol was removed by adding 150 μl of 24:1 chloroform:isoamyl alcohol solution, 5 min 16,000×G. The upper aqueous phase was transferred to 1.5 ml tube. Another 100 μl of EB buffer (Qiagen) were added to Phase-Lock Gel Heavy 1.5 ml tube to collect the remaining upper phase and transferred to the same 1.5 ml tube with the rest of the upper phase. After adding 40 μl of 3M Sodium Acetate, 1.5 μl of GlycoBlue reagent (Life technologies) and 800 μl ethanol, samples were incubated on −80° C. for at least 30 min. Samples were centrifuged 15 min 12,000×G 4° C. Sup removed and pellets washed 1×0.5 ml 70% ethanol, 5 min 12,000×G. Samples were eluted with 50 μl EB buffer (Qiagen). 1 μl of DNAse-free RNAse A (Sigma, 37 mg/ml) was added to every sample including input samples, 30 min 37° C. Then, 1 μl (20 μg) Proteinase K (Roche) were added and samples incubated for 1 hr on 55° C. and 16 hrs on 65° C. to reverse cross-linking. Then, samples were supplemented to 300 μl with EB buffer. Phenol:chloroform:isoamyl alcohol extraction and ethanol precipitation were repeated exactly the same way it was performed in the first step. Samples eluted with 50 μl EB buffer (100 μl EB buffer for input samples). qPCR assays were performed on CFX96 real-time PCR system (Bio-Rad). Specific primers are listed in Table a. Threshold cycle values were translated into initial template amount for each sample based on the standard curve prepared from known quantities of genomic DNA. Enrichment of specific PCR amplicons was expressed as percentage of total input DNA for each reaction.

Protein Expression and Purification

Mouse CBX7 carrying Flag and HA tag on C-terminus was expressed in Sf9 insect cells using the bac-to-bac system (Invitrogen). Protein extract was prepared by resuspending cell pellet in lysis buffer F (20 mM HEPES-KOH [pH 7.9], 300 mM NaCl, 4 mM MgCl₂, 1 mM DTT, 20% glycerol, and protease inhibitors mix [Sigma]). After 15 strikes with tight pestle on 15 ml Dounce homogenizer, cell suspension was supplemented with 0.1% Nonidet-P-40, 0.2% Triton-X-100, 5 u/ml TurboDNAse (Invitrogen) and 12.5 μg/ml Heparin. After 30 min 4° C. incubation on orbital shaker, cell lysate was subjected to 2 rounds of 15 min 30,000×G centrifugation on 4° C. Supernatant was collectedand snap-frozen in liquid nitrogen. M2 anti-FLAG beads (Sigma) were used for all purifications. Proteins were bound to M2 beads in lysis buffer for 2 hr at 4° C. Beads were washed twice with buffer F (500 mM NaCl), twice with buffer F (300 mM NaCl) and twice with elution buffer (50 mM Tris pH 7.4, 100 mM NaCl). Proteins were eluted twice by 1 hr incubations with 0.2 μg/ml 3×-FLAG peptide (Sigma). Protein concentrations were determined by SDS-page and Bradford assay using bovine serum albumin as a standard.

Electrophoretic Mobility Shift Assays

RNA-EMSA assays with CBX7 protein were performed as follows. Labeled RNAs were produced with MEGAscript® T7 Transcription Kit (Life Technologies) and purified from 6% acrylamide TBE-urea gels. Labeled RNAs were prefolded in buffer TE+300 mM NaCl by incubating for 2 min at 95° C., followed by 20 min incubation on ice. Binding reactions were assembled with 20 μl of binding mix (13 mM Tris pH 8.0, 0.2 mM EDTA, 68.8 mM NaCl, 20% Glycerol, 0.2 mg/ml Yeast tRNA, 4 mM DTT, 4 μl 2500 cpm/μl RNA probe). LNA oligonucleotides were added to binding mix at final concentration of 8 μM and samples were pre-incubated on ice for 10 min. After pre-incubation, binding mix samples were combined with 60 μl of purified protein in dialysis buffer (50 mM Tris pH 7.4, 5 mM MgCl₂, 50 mM NaCl, 1 mM DTT, 10% Glycerol, 4 u/μl Protector RNAse inhibitor (Roche)). Control experiments were performed with dialysis buffer only or control proteins—Flag-GFP or GST-Flag-HA, dissolved in dialysis buffer at the highest protein concentration in the particular experiment. After 30 min on ice, the sample was loaded onto a 5% 37:1 acrylamide (Bio-Rad) gel in 0.5× TBE buffer (45 mM Tris-Borate, 1 mM disodium EDTA) and run for 90 min at 250 V at 4° C. Gels were exposed to phosphorimager screens. For validation of motif sequences, labeled RNA probes were produced with MEGAshortscript™ T7 Transcription Kit (Life Technologies) and gel purified using 15% TBE-urea gels (Life technologies) Similar RNA-EMSA conditions were applied except 8% 37:1 acrylamide (Bio-Rad) gels replaced 5% gels. Sequences of RNA probes are given in Table a.

Western Blotting

20 μl of protein extracts were resolved on 4%-20% gradient SDS-PAGE gels (Bio-Rad) and proteins were transferred for 1 hr on 100V in transfer buffer (48 mM Tris, 39 mM Glycine, 20% methanol) to Immobilon-P 0.45 μm PVDF membrane (Millipore) using Mini Protean Tetra transfer unit (Bio-Rad)). To detect CBX7 protein expression, Western blotting was performed with mouse monoclonal CBX7 Antibody (G-3) (Santa Cruz Biotechnologies, sc-376274) as primary antibody and goat-anti-mouse-HRP (Promega) as a secondary antibody. For quantitative Western blotting of DCAF12l1 protein, anti-WDR40B (Dcaf12l1) rabbit polyclonal antibody (Biorbit, orb155395) was used as a primary antibody along with anti-Ctcf rabbit polyclonal antibody (Cell Signaling Technologies, #2899) as a loading control. Goat-anti-rabbit-HRP (Promega) was employed as a secondary antibody. Protein bands were developed using Western Lightening Plus-ECL Kit (Perkin-Elmer) and the signal intensity was analyzed using Chemidoc MP Imaging System (Bio-Rad) and ImageLab Ver. 5.2.1 software (Bio-Rad). Exposures were captured on different times using ChemiDoc cumulative signal option to avoid signal saturation. Standard curves were prepared using increasing amounts of cell extract (FIG. 6G), to confirm a signal intensity staying in a dynamic linear range.

Quantification and Statistical Analysis of qPCR Data

Data represents the average±standard deviation for at least 3 biological replicates as stated in the figure legends. P values were determined by unpaired two-tailed student t-test unless otherwise stated.

Quantitative Analysis of RNA-EMSA

Gels were exposed to phosphoimager screens and scanned using Typhoon laser scanner (GE Healthcare). Radioactive signal intensity was quantified by Image Quant 5.2 software (GE Healthcare). Fraction of bound RNA (signal intensity of the shifted bands divided by the total signal intensity in the particular lane) was computed for every protein concentration and plotted against corresponding protein concentration. To determine dissociation constant (Kd), the resulting binding curves were fitted to sigmoidal plots by non-linear regression using “Prism” software (Graphpad Software inc).

Analysis of CLIP-Seq Data

Libraries were subjected to high-throughput sequencing using Illumina HiSeq 2000 apparatus according to manufacturer instructions. Approximately 40 million paired-end 50 bp reads were generated per every CLIP-seq sample. Adaptor sequences were trimmed with either Trim Galore! V0.3.3 (http://www.bioinformatics.babraham.ac.uk/projects/trim_galore/) (for CLIP-seq; stringency 15 and allowed error rate 0.2), or cutadapt (v1.0) (https://pypi.python.org/pypi/cutadapt/) Identical genomic sequences (PCR duplicates) were removed by custom program prior to alignment. To account for the M. mus (mus)/M. castaneus (cas) hybrid character of mouse EL 16.7 ES cell line that was employed in a CLIP-seq studies, reads were first aligned to custom mus/129 and cas genomes, and then mapped back to the reference mm9 genome (Pinter et al., 2012). All alignments were performed by utilizing Tophat (v2.0.11) (Trapnell et al., 2012). Post-processing of alignments was performed with custom scripts using SAMtools (Li et al., 2009), and BEDtools v2.17.0 (Quinlan and Hall, 2010). These included accounting, alignment file-type conversion, extracting and reads sorting (SAMtools), and obtaining wig coverage files (SAMtools depth).

Fragment per million (fpm) wig files were then created by scaling uniquely aligned wig files to total number of fragments per million in each library (determined by SAMtools flagstat combining reads “with itself and mate mapped” and “singletons”). CLIP-seq enriched tag density wig files were viewed in UCSC genome browser (Kent et al., 2002) or Integrated Genome Browser (IGB) (Nicol et al., 2009). Then consecutive wig entries of equal coverage were merged forming bed files that were used for peak calling. The peak caller software PeakRanger (v.16) (Feng et al., 2011) was used. The software requires an even distribution of watson/crick entries, thus, prior to peak calling, strand specific bed file entries were randomized per each strand. PeakRanger was called with arguments ranger -p 0.01-format bed -gene_annot_file (mm9), -d experiment and -c mock-transfected control, to identify narrow peaks with p-value 0.01 or less.

For assessing dCLIP fragments footprints PeakRanger-enriched CLIP fragments from 3 libraries were pooled and merged in a strand-specific manner to create continuous CLIP fragments (in case of overlapping peaks). Length-frequency histogram of enriched CLIP fragments was obtained, along with mean, median, and SD.

RNA-Seq Analysis

For RNA-seq, RNA was extracted from cells used for dCLIP experiments. Starting amount of Total RNA was 4 μg. RNA was depleted of ribosomal RNA using Ribominus kit (Life technologies). Strand-specific cDNA libraries were constructed using Superscript III reverse-transcriptase for first-strand synthesis, NEBNext mRNA Second Strand Synthesis Module supplemented with dUTP (NEB) for second-strand synthesis, and NEBNext ChIP-Seq Library Prep Master Mix Set for library preparation. Libraries were subjected to high-throughput sequencing using Illumina HiSeq 2000 apparatus according to manufacturer instructions. Approximately 40 million single-end 50 nt reads were generated for every RNA-seq sample. Data processing was performed essentially as described previously (Kung et al., 2015). Adaptor sequences were trimmed from libraries with Trim Galore! v0.3.3 (for dCLIP-seq and RNA-seq; stringency 15). PCR duplicates were removed by custom programs prior to alignment. To account for the M. mus (mus)/M. castaneus (cas) hybrid character of the ES cell lines, reads were first aligned to custom mus/129 and cas genomes, and then mapped back to the reference mm9 genome. RNA was aligned with Tophat (v2.0.8 or greater). Post-processing of mm9 alignments was performed with custom C and Perl programs and bash shell scripts, SAMtools v0.1.18, and BEDtools v2.17.0.

RNA-Seq vs CLIP-Seq Analysis

For RNA-seq analysis two CBX7 libraries were used and for CLIP-seq analysis three CBX7 libraries were used. We have performed the following analysis for both RNA-seq and CLIP libraries: By applying the Homer (http://homer.salk.edu/homer/motif/) suit's makeTagDirectory and makeUCSCfile algorithms we converted aligned SAM files into strand-specific bedGraph files. Reads were further filtered to eliminate mappable reads assigned to ribosomal DNA and mitochondrial DNA, and per each library read counts values were normalized to the corresponding 3^{rd} quartile read counts value. Strand-specific wig files were then binned to 100 bp windows and subjected to Piranha peak analysis (http://smithlabresearch.org/software/piranha/) resulting in significantly (p<0.01) enriched peaks. Piranha CLIP peaks were further filtered to include only peaks that were considered enriched also based on the PeakRanger algorithm (see “PeakRanger” peak calling under STAR Methods' “Analysis of CLIP-Seq data” section). To compare between the resultant enriched CLIP signals and their corresponding enriched RNAseq signals, piranha peaks files (of both, CLIP libraries and RNAseq libraries) were processed by the Homer's makeTagDirectory algorithm, and subsequently parsed to genomic features by the Homer's analyzeRNA algorithm, generating a matrix of total read counts per gene normalized to the length of each gene. Next, a matrix holding only genes that manifested CLIP signals higher than zero in at least two out of three datasets was created. The log 2-transformed normalized read values of each of 2 or 3 CLIP libraries that had enriched signals were averaged to reflect an averaged CLIP signal per gene. The normalized values (per gene) of corresponding RNAseq libraries, similarly analyzed in parallel, were averaged in the same manner to reflect an average RNAseq signal per gene. This analysis resulted in a matrix containing 1,333 genes with positive CBX7 CLIP signal.

To focus on a gene cohort that represents genes with high CLIP and RNAseq signals we selected 10% of the CLIP'ed genes (135 genes circled by green ellipse, as shown in FIG. 1G. List of 135 genes is provided in Table d). We also identified all genes (6,671 genes) that manifested no piranha CLIP peaks in all three datasets, and that showed no PeakRanger CLIP peaks in at least two out of three datasets. This group of CLIP-devoid genes was further filtered to include only genes (2,078) that their average RNAseq signal corresponded to the same RNAseq signal range manifested by the 135 highly CLIP'ed genes (this gene group is indicated by red-colored dots on the scatter-plot as shown in FIG. 1G. Note that all genes within this group are devoid of CLIP signal (CLIP signal equals to zero) and their plotting at the bottom of the scatter plot (after replacement of zero values by a “dummy” value) was generated merely for representation purpose (FIG. 1G).

TABLE d
Related to FIG. 1G. List of 135 CBX7 high-binding transcripts.

| GeneID | GeneSymbol |
|---|---|
| NR_028540 | Snord12 |
| NR_002142 | Rpphl |
| NR_002865 | Rnu11 |
| NR_028572 | Snora43 |
| NR_031758 | Snora26 |
| NR_030451 | Mir682 |
| NR_037683 | Snord42b |
| NM_010106 | Eef1a1 |
| NR_030762 | Snord17 |
| NM_011401 | Slc2a3 |
| NM_007907 | Eef2 |
| NR_027885 | Vaultrc5 |
| NM_012053 | Rpl8 |
| NM_177099 | Lefty2 |
| NR_015531 | Dancr |
| NM_010240 | Ftl1 |
| NR_038063 | Rplp2-ps1 |
| NR_045289 | Rab26os |
| NM_018860 | Rpl41 |
| NM_001145804 | Nucks1 |
| NR_110499 | Rpl14-ps1 |
| NM_008774 | Pabpc1 |
| NM_024212 | Rpl4 |
| NM_029352 | Dusp9 |
| NM_012052 | Rps3 |
| NM_001078167 | Srsf1 |
| NM_152806 | Ddx17 |
| NR_003363 | Gm6548 |
| NM_009128 | Scd2 |
| NM_010202 | Fgf4 |
| NM_018796 | Eef1b2 |
| NM_008143 | Gnb2l1 |
| NM_029872 | Hnrnpa0 |
| NM_026147 | Rps20 |
| NM_001289828 | Nanog |
| NM_026242 | Mrfap1 |
| NM_010094 | Lefty1 |
| NM_018853 | Rplp1 |
| NM_021278 | Tmsb4x |
| NM_011562 | Tdgf1 |
| NM_016906 | Sec61a1 |
| NM_029767 | Rps9 |
| NM_010239 | Fth1 |
| NM_026055 | Rpl39 |
| NM_029701 | Spcs3 |
| NM_007451 | Slc25a5 |
| NM_007475 | Rplp0 |
| NR_027901 | 2900060B14Rik |
| NM_011712 | Wbp5 |
| NM_019419 | Arl6ip1 |
| NM_009127 | Scd1 |
| NM_001204875 | Set |
| NM_010480 | Hsp90aa1 |
| NM_026155 | Ssr3 |
| NM_181401 | Tmem64 |
| NM_198006 | Coa5 |
| NM_172665 | Pdk1 |
| NM_001081164 | Otud4 |
| NM_009786 | Cacybp |
| NM_001039129 | Hnrnpa1 |
| NM_008468 | Kpna6 |
| NM_001190800 | Ddx19b |
| NM_026036 | Cmtm6 |
| NM_026521 | Zfp706 |
| NM_024166 | Chchd2 |
| NM_008019 | Fkbp1a |
| NM_008972 | Ptma |
| NM_001252260 | Npm1 |
| NM_001033474 | Atxn7l3b |
| NM_001081005 | 1500012F01Rik |
| NM_001253857 | Tet1 |
| NM_001285412 | Calu |
| NM_025586 | Rpl15 |
| NM_145625 | Eif4b |
| NM_013725 | Rps11 |
| NM_001171035 | Tmbim6 |
| NM_011400 | Slc2a1 |
| NM_175403 | Mlec |
| NM_007984 | Fscn1 |
| NM_023755 | Tfcp2l1 |
| NM_001136069 | Ldha |
| NM_008568 | Mcm7 |
| NM_146012 | Ctdsp2 |
| NM_011296 | Rps18 |
| NM_013765 | Rps26 |
| NM_001134427 | Cdv3 |
| NR_027907 | AI414108 |
| NM_007478 | Arf3 |
| NM_020600 | Rps14 |
| NM_008251 | Hmgn1 |
| NM_026517 | Rpl22l1 |
| NM_009546 | Trim25 |
| NM_001142809 | S1c6a8 |
| NM_001159375 | Eif4a1 |
| NM_001293559 | Cox4i1 |
| NM_007748 | Cox6a1 |
| NM_009391 | Ran |
| NM_001030307 | Dkc1 |
| NM_028451 | Larp1 |
| NM_019647 | Rpl21 |
| NM_033561 | Eif4h |
| NM_008810 | Pdha1 |
| NR_002883 | Gm5643 |
| NM_009536 | Ywhae |
| NM_010193 | Fem1b |
| NM_033617 | Atp6v0b |
| NM_011404 | Slc7a5 |
| NM_024214 | Tomm20 |
| NM_009951 | Igf2bp1 |
| NM_009320 | Slc6a6 |
| NM_001253757 | Anp32e |
| NM_001164806 | Bend4 |
| NM_025881 | Luc7l |
| NM_178627 | Poldip3 |
| NM_011292 | Rpl9 |
| NM_001252292 | Mest |
| NM_011291 | Rpl7 |
| NM_001110499 | Canx |
| NM_144866 | Etf1 |
| NM_001004153 | AU018091 |
| NM_145833 | Lin28a |
| NM_016898 | Cd164 |
| NM_172467 | Zc3havl1 |
| NM_001142732 | Ttll3 |
| NM_133815 | Lbr |
| NM_001190718 | Dcaf12l1 |
| NM_001289599 | Txndc5 |
| NM_178111 | Trp53inp2 |
| NM_007589 | Calm2 |
| NM_011462 | Spin1 |
| NM_028261 | Rian |
| NM_153592 | Erlin2 |
| NM_045170 | Gm10336 |
| NM_021383 | Rqcd1 |
| NM_001276481 | Dag1 |

To determine reproducibility among dCLIP peaks, we utilized deepTools (Ramirez et al., 2014) analysis, we averaged the significance values (−log(p-value)) of strand-specific peaks enriched in at least two out of three replicates, per bin. 1-kb bin size was applied. Pairwise-Pearson correlation (PPC) analysis was performed for the 3 replicates. Scatter plots were generated (FIG. 11B) and Pearson correlation coefficient was calculated per pair. Overall positive correlation was observed with Pearson correlation coefficients ranging from 0.44 to 0.67 (FIG. 11B).

We further utilized a matrix of total read counts per gene normalized to the length of each gene (as described above), and per all genes that manifest CLIP signal in at least two replicates, conducted three paired comparisons as follows: Replicate #1 vs. Replicate #2; Replicate #1 vs. Replicate #3; Replicate #2 vs. Replicate #3. Normalized data points were plotted and correlative patterns are presented as three scatterplots (FIG. 11B). Spearman's and Pearson's correlation coefficients were calculated per each comparison, indicating a high concordance between three CLIP replicates, with average Spearman's correlation coefficient of 0.87, and Pearson's correlation coefficient of 0.89.

To characterized and summarize whole-genome occupancy pattern of peaks, we pooled and merged all piranha peaks (overlapping PeakRanger peaks) from three libraries into one continuous track (containing 8,578 peaks), and employed CEAS analysis (Ji et al., 2006; Shin et al., 2009), using the mm9 KnownGenes database, and as background dataset we used a merged transcriptome coverage track obtained from two RNA-seq experiments conducted in the same cell line that was employed for conducting CLIP experiments.

Chip-Seq Analysis

Data processing was performed as described previously (Kung et al., 2015). Normalization to input libraries was performed with window size 500 and step size 100. To obtain highly significant ChIP peaks, we used the software macs2 (version 2.1.0.20150603) (Zhang et al., 2008) with highly stringent constraints (p 0.01) to identified ChIP peaks verses input for the IP. Additionally, we compared the IP verses a control IP using PeakSeq (version 1.3) (Rozowsky et al., 2009) with stringent constraints (Enrichment_fragment_length 200, Enrichment_mapped_fragment_length 200, Background_model Simulated, max_Qvalue 0.1, target_FDR 0.05, N_Simulations 10, Minimum_interpeak_distance 200). Then we constrained the macs called ChIP peaks to only those that were also called against the control experiment. Finally, we limited the resulting peaks to those that intersect with IP enriched regions six-fold over the input. IP and input regions are obtained using smoothed coverage data using a 500 nucleotide window with 100 nucleotide steps. To assess the relationship between CBX7s binding to RNA vs. DNA we counted the number highly significant ChIP peaks that overlapped dCLIP-bound transcripts versus all expressed transcripts using non-parametric techniques (1,000 random selections with replacement). Overlap was counted if ChIP peak was located inside an open reading frame or a promoter region (2,000 nt upstream to transcription start site) of the corresponding transcript.

Motif Analysis of CLIP-Seq Data

Basing on three separate CLIP experiments that were performed, three mouse CBX7 CLIP-seq datasets were raised, containing the following numbers of PeakRanger enriched CLIP regions aligned to positive and negative strands, respectively (after excluding rDNA and mtDNA sequences): #1: (4,262, 4,225), #2: (4,254, 3,979), #3: (5,021, 5,009). To thoroughly analyze the biological information embedded within the three independent CBX7 CLIP-seq libraries of mouse we have defined three grouping categories on the basis of regions redundancy (overlapping) existing between the three independent libraries. We dubbed these categories: (1) “Individuals”: a category containing original, unfiltered, enriched CLIP regions; (2) “OneOL”: a category containing enriched CLIP regions that their span intersects with the span of at least another enriched CLIP region that was raised from another independent library; (3) “TwoOL”: a category composed of enriched CLIP regions that their span intersects with the span of enriched CLIP regions raised from two independent libraries. This approach was based on the presumption that CBX7 could have more than one consensus motif and that each library may not have sufficient depth to capture all CBX7-binding sites. Basing on these three categories we opted to take a parallel branched approach by classifying the enriched regions raised from the three independent CBX7 CLIP-seq libraries into nine datasets, namely: Individuals #1, Individuals #2, Individuals #3, OneOL #1, OneOL #2, OneOL #3, and TwoOL #1, TwoOL #2, TwoOL #3. In addition to identifying the boundaries of enriched CLIP regions, PeakRanger algorithm determines the summit position of each region (harboring the topmost CLIP signal), which manifests the strongest binding affinity towards CBX7. Thus, in order to pinpoint the most significant CBX7-RNA binding events we referred to the summit point of each enriched region as an anchoring position and stretched a 100 bp region around it (±50 bp) (Ma et al., 2014). Per each of the nine datasets, summit-based 100 bp CLIP-enriched regions of positive and negative strands were combined into a single batch, resulting the following (number of enriched CLIP regions is indicated in parenthesis): (1) Individuals #1 (8,492); (2) Individuals #2 (8,237); (3) Individuals #3 (10,031); (4) OneOL #1 (3,422); (5) OneOL #2 (2,499); (6) OneOL #3 (3,182); (7) TwoOL #1 (1,125); (8) TwoOL #2 (1,083); (9) TwoOL #3 (1,088). In each of the nine datasets, enriched CLIP regions were sorted based on their FDR significance values as defined by PeakRanger. In order to discover novel binding motifs that may be enriched in each of the nine CLIP-seq datasets we employed MEME-ChIP tool that employs both, the MEME and DREME algorithms for identifying de-novo binding motifs (Bailey et al., 2009; Ma et al., 2014). Since the MEME-ChIP tool functions most efficiently when introduced with datasets containing up to 600 sequences (Ma et al., 2014), and due to the fact that 6 of our 9 datasets were 4-17 fold larger, we created a pipeline that receives a large-sized dataset of enriched CLIP regions, splits it into equal-sized batches (typically of 600 sequences per batch), and then, in parallel per each of the batches, fetched with Bedtools (Quinlan and Hall, 2010), the strand-specific FASTA sequences (100 bp around the summit point of each enriched region), and executes the MEME-ChIP tool in strand-specific mode (“-norc”). Given that enriched CLIP regions within each of the CLIP-seq datasets manifest a wide range of significance (FDR) values, each of the large-sized datasets was split based on equal-sized intervals across the FDR-sorted dataset, allowing an overall balanced representation of significance values of CLIP regions throughout all batches. Thus, the three “Individuals” CLIP-seq datasets (#1, #2, #3) were processed as 14, 13, and 16 batches, respectively, whereas the three “OneOL” CLIP-seq datasets (#1, #2, #3) were processed as 5, 4, and 5 batches, respectively. Each of the three “TwoOL” CLIP-seq datasets (containing 1,100 enriched regions) was processed as one batch.

Per each of the analyzed CLIP region batches MEME-ChIP tool determined the enrichment of several binding motifs. All novel motifs identified under each of the three categorical groups, namely, “Individuals”, “OneOL”, or “TwoOL”, were pooled together, yielding motif pools of 158, 48, and 19 motifs, respectively. Next, all de-novo motifs identified under each categorical group were subjected to multiple motif alignment analysis employed by the similarity-clustering tool, STAMP (Mahony and Benos, 2007). In each case, STAMP analysis, employed in strand-specific mode (“-forwardonly”), generated a phylogenetic newick tree that was constructed by comparing strand-specific similarity of binding motifs. Phylogenetic Newick trees were then depicted by employing the Molecular Evolutionary Genetics Analysis (MEGA) software (Tamura et al., 2013). In addition, our pipeline employed “SeqLogo” Bioconductor package (Bembom O. seqLogo: Sequence logos for DNA sequence alignments. R package version 1.34.0.) for generating a sequence logo for each of the enriched binding motifs. Next, we viewed the Newick tree of each of the categorical groups, and based on its branch structure grouped together neighboring motifs that share pattern similarity. We then re-subjected each of the groups containing similar motifs to STAMP analysis that generated a unique generalized FBP (Familial Binding Profile) model reflecting the general profile of all binding motifs within each group. FBP analysis was performed redundantly for each group—“Individuals”, “OneOL”, and “TwoOL”. The “individuals” FBPs were seen to fall within FBPs identified by the “OneOL” and “TwoOL” groups, strongly suggesting that the motifs from “Individuals” datasets (obtained from a single library) resembled those arising from the OneOL and TwoOL (more inclusive) datasets. Altogether, STAMP analysis of the three categorical groups yielded 24 FBPs, namely, 10 “Individuals” FBPs, 7 “OneOL” FBPs, and 7 “TwoOL” FBPs (FIG. 3A,B). Indeed, the results of this computational survey, performed separately in each of the categorical datasets, suggested that enriched motifs of all three datasets—including the “individuals”—were highly redundant and shared high sequence similarity. Using STAMP, the FBPs could be clustered into 4 higher-order motif families (hereafter dubbed “FAMs” for “FBP Association Module”), each being distinct and representing a consensus for each family

To statistically analyze over-representation of 24 mouse CBX7 FBPs in each of the three original mouse CBX7 libraries, we first assembled a motif library of 24 position weight matrices (PWMs) by combining the all FBPs from three datasets. To enable further downstream the tracking of dataset that originally yielded each of the de-novo FBPs, in addition to being labeled by a serial number, FBPs were labeled as either “Indiv.”, “OneOL”, or “TwoOL”.

By utilizing Bedtools, we fetched, per each of the three CLIP libraries, the FASTA sequences of the enriched CLIP region. Next, we used CLOVER (Frith et al., 2004) at a strand-specific mode (−z=1) to detect binding motifs that were enriched in mouse CBX7 CLIP regions. Per each of the three CLIP libraries CLOVER determined the statistical enrichment of each mouse FBPs relative to two background sets that were constructed from the entire transcriptome coverage obtained from two separate RNA-seq experiments conducted in the same cell line that was employed for conducting CLIP experiments. Each of the reported binding motifs was given a score (“raw score”) based on its predicted binding energy, and two p-value significance scores, each corresponding to the one background file (Frith et al., 2004). FBPs with raw scores higher than 6 and two significant enrichment score (p≤0.05) were selected for further analysis.

In addition, we assembled a library of 1,179 Known PWMs, by combining the RNA-binding motifs in the compendium of RBP recognition motifs (Ray et al., 2013), together with DNA binding motifs in the JASPAR database (Sandelin et al., 2004), and those in recently reported sets of PWMs for mouse transcription factors (Badis et al., 2009) (Wei et al., 2010; Xie et al., 2010). By applying the same parameters used for detecting enrichment of FBPs, we employed CLOVER for identifying enrichment of known motifs within the CBX7 CLIP regions. Per each of the enriched FBPs and Known motifs we summarized the number of binding sites hits identified within each library, and by dividing this number by the total number of library's CLIP regions, we obtained a “prevalence score” for each of the FBPs and Known motifs. We combined the output parameters obtained from CLOVER analysis of three CLIP libraries into one database, and sorted FBPs and Known motifs based on four scoring criteria: (1) Number of libraries in which a motif was significantly enriched (p≤0.05); (2) Average significance score (p-value); (3) Average prevalence score; (4) Average raw score. We discard all FBPs and Known motifs that manifested an inverse significance relative to background datasets (p≥0.95) in at least one dataset, and all motifs that their average prevalence score was under 5%. Based on this sorting procedure all qualified motifs were ranked (with motif #1 represent the motif with the best scores). Altogether, 11 FBPs (out of the originally introduced 24 mouse FBPs), and 80 Known motifs were met our criteria and found significantly enriched in at least one of the mouse CBX7 CLIP datasets (for known motifs a literature survey that determined their previously suggested role in RNA metabolism and function was additionally implemented as part of the filtration procedure). Among the 11 qualified mouse FBPs, 8 were significantly enriched in 3 CLIP libraries, while 3 were enriched in 2 libraries. Among 80 known motifs, 29 (36%), 29 (36%), and 22 (27%) motifs were enriched in three, two, and single CLIP libraries, respectively. Interestingly, 53 of these enriched Known motifs were RNA-binding motifs that previously reported as part of the compendium of RBP recognition motifs (Ray et al., 2013).

In order to determine whether specific FBPs could be grouped together into a higher-ordered motif family, also dubbed as “FAM” (FBPs Association Module), by employing STAMP analysis over the 11 qualified mouse FBPs we obtained a phylogenetic tree that identified the presence of four highly-ordered FAMs, which we named: FAM1 (composed of FBP2_Indiv., FBP5_TwoOL, FBP7_OneOL, FBP2_TwoOL); FAM2 (composed of FBP4_TwoOL, FBP3_TwoOL); FAM3 (composed of FBP5_OneOL, FBP9_Indiv.); FAM4 (composed of FBP7_TwoOL, FBP10_Indiv., FBP6_OneOL).

Basing on two separate CLIP experiments that were performed, two human CBX7 CLIP-seq datasets were raised, containing the following numbers of PeakRanger enriched CLIP regions aligned to positive and negative strands, respectively (after excluding rDNA and mtDNA sequences): #1: (2,552, 2,125), #2: (399, 490). As described above, Peak summits were used as anchoring positions for stretching 100 bp strand-specific regions around them (±50 bp). By applying identical computational tools and similar analytic steps as these described above for mouse CBX7-CLIP, we carried out identification of de novo binding motifs of Human-CBX7. Altogether, this analysis yielded 122 de novo binding motifs that were subsequently subjected to STAMP similarity-clustering analysis (see above), generating 27 human FBPs. We utilized CLOVER for determining the statistical enrichment of each of 27 human FBPs, in addition to 1,179 Known PWMs (see above) relative to two background sets that were constructed from the transcriptome of human HEK-293 cells. After filtering out motifs that were insignificantly enriched (p>0.05), or manifested presence lower than 5%, we obtained 9 human FBPs, and 50 Known motifs that met our criteria. Next, we utilized STAMP analysis (see above) for identifying motif similarities among 9 enriched Human FBPs and 11 enriched mouse FBPs. In parallel, we carried out STAMP matching analysis between 9 enriched Human FBPs and 50 Known binding motifs (Ray et al., 2013).

To define the global distribution of each of the four FAMs we extracted from CLOVER output data files of each of the three CLIP libraries, the genomic coordinates of all 11 qualified FBPs, grouped by FAMs. We then employed CEAS analysis (Ji et al., 2006; Shin et al., 2009), using the mm9 KnownGenes database, and as background dataset we used a merged transcriptome coverage track obtained from two RNA-seq experiments conducted in the same cell line that was employed for conducting CLIP experiments.

FAM-Occupancy in CLIP Regions Compare to Their Corresponding Full-Span Genomic Features

In order to determine the potential contribution of each of the four FAMs to transcripts binding to CBX7, we first pooled together the CLOVER output data of all three CLIP libraries and grouped them by FAM. By employing R packages (“GenomicFeatures”, “Bsgenome.Mmusculus.UCSC.mm9”, and NCBI37/mm9 knownGenes genome assembly), we extracted coordinates of genomic features (3′UTR, 5′UTR, coding sequences (CDs), and introns). We then annotated all FBPs (FAMs) overlapping with mouse genes to their corresponding genomic features. Next, we calculate per each CLIP transcript its “FAM occupancy score”. To this end, we aggregated per each gene, and per a given genomic feature, all FAM-hits that were detected within each of the transcripts that were obtained by CLIP. In case that the genomic feature was composed of multiple frames per a single gene (such in the case of introns that composed of multiple frames per a single gene), FBP-hits were aggregated from all frames that were retrieved by CLIP. Then, by dividing the total number of FAM-hits identified at a given CLIP fragment by the length of the genomic feature that CLIP FAM resides in, we generated per each gene a “CLIP-associated FAM-occupancy score”. Next, we calculated a “full-length genomic feature-associated FAM-occupancy score”. To this end, per each of the transcripts retrieved by CLIP, we mapped all putative FAM-hits across the entire span of the genomic feature. Thus, per a given genomic feature we mapped all real FAM-hits (overlapping with regions obtained by CLIP) in addition to predicted FAM-hits (excluded from regions obtained by CLIP) that reside within the full span of a genomic feature. Finally, we calculate “FAM-occupancy Ratio” per each CLIP transcripts by dividing CLIP FAM-occupancy score by genomic feature-associated FAM-occupancy score.

We summarized the results of this analysis by as a series of boxplots that describe the distribution of the FAM-occupancy ratio within the four FAM groups across each of the tested genomic features. The analysis indicated that FAM2, when integrated within CLIP transcripts provides in general higher potency for transcripts to bind CBX7, as compare to than all other FAMs. This potency of FAM2 was observed across all tested genomic features.

Analysis of FAMs that Reside within the Same CLIP Fragment

We noticed that some CLIP transcripts harbor more than one FAM per a fragment (as indicated by count histogram of number of FAMs residing adjacently on the same dCLIP fiber; FIG. 3C). For analyzing these events, CLIP fragments from 3 libraries were pooled and merged to create continuous CLIP fragments. Then, by utilizing bedtools, we identified all paired FAMs residing on the same CLIP fragment, and calculated the distance between their centers. We identified all permutations of FBP pairs residing on the same CLIP fragment, and in all cases of hetero-pair (such as “FAM1-FAM2”) we distinguished between the two possible orientations, referring to all cases in which FAM2 was located downstream to FAM1 as “FAM1-FAM2_Dnstr”, and to all cases in which FAM2 was located upstream to FAM1 as “FAM1-FAM2_UpStr”. In all cases in which homo-pair were identified, for example as in the case of FAM1 residing in proximity to FAM1, the pair was reported as “FAM1-FAM1_DnStr”. We consider all FAM pairs separated by a distance of less than 6 bp as a single site, thus all reported FAM pairs resulted from this analysis were above 6 bp. We then annotated each FAM pair to the genomic feature and reported all non-annotated pairs as “No Feature”. We plotted all FAM-pairs distances as boxplots and designated by yellow dots their corresponding average in each dataset. In addition, per each of the FAM pairs, we reported in a barplot, all FAM-pair counts, grouped according to their genomic feature annotation. Since the different FAMs were represented in the entire CLIP dataset in different ratios, for example, FAM1 had much more hits at CLIP fragments compare to all other FAMs, we also reported in a barplot all the relative ratios of each FAM-pair relative to the total abundance of the FAMs that are composing it. Thus, for example In the case of the pair FAM1-FAM2 in 3UTR, we calculated the following equation in order to obtain the relative FAM-pair likelihood percentage: (count of FAM1-FAM2 in 3UTR)/(count of FAM1 in 3UTR)*(count of FAM1-FAM2 in 3UTR)/(count of FAM2 in 3UTR)*100. The analysis indicated that FAM1-FAM1 pairs have the highest likelihood to create a pair within the same CLIP fragment.

In the scope of a separated analysis we counted per all four FAMs that number of appearances within each genomic feature or outside any genomic feature (“No Feature”). We plotted these counts as a barplot, grouped by FAM type, and according to genomic features.

To assess the contribution of FAMs co-clustering on the same dCLIP transcript we split CLIP fragments into two batches, namely: Single FAMs per CLIP fiber (FAMs with zero adjacent FAMs on the same CLIP fiber), and Multiple FAMs per CLIP fiber (FAMs with one or more adjacent FAMs on the same CLIP fiber). Then, we analyzed separately the FAM-likelihood ratios per each of these two batches, for each of the four FAMs, within each genomic feature.

Metagene Analysis of FAM Pairs

To determine whether FAMs have a tendency to reside next to each other as pairs in preferential manner, we plotted per each of the four FAMs its distribution of distances from its center to the center of its paired FAM. We conducted this analysis on a single bp resolution, across a window of ±200 bp (X-axis), presenting the count number for each FAM-pair on the Y-axis.

Metagene Analysis of FAM Sites for Profiling icSHAPE Signals

To determine whether CBX7 CLIP transcripts may adopt specific secondary RNA structures, we took advantage of the publically available RNA structural signatures established via in vivo and in vitro click selective 2′-hydroxyl acetylation and profiling experiments (icSHAPE). The GRCm38/mm10 bigwig data files corresponding to in vivo and in vitro icSHAPE structural profiles were obtained from GEO database (record GSE60034) (Spitale et al., 2015), and converted to the NCBI37/mm9 assembly by employing UCSC tools, bigWigToBedGraph following by liftOver. Using in house codes, we calculated separate metagene structure profiles around an anchor position that was defined as the center of each of four FAM binding motifs. Individual structural profiles of in vivo and in vitro icSHAPE scores were generated, at single nucleotide resolution, by accumulating all icSHAPE scores detected within the limited scope of 25 nucleotides upstream and downstream. The average icSHAPE profile was then generated by division of the accumulative icSHAPE score per single nucleotide by the total number of ±25 bp FAM regions containing a total icSHAPE score higher than zero (>0). Thus, 50 bp regions that harbor no icSHAPE signal around the center of FAM motif were excluded from this analysis. For contrasting the profiles of FAM motifs that were identified in CLIP regions (“Real FAMs”), against a control cohort of FAM motifs that were not identified in CLIP regions (“Predicted FAMs”), we took advantage of the previously established motif binding sites database that contains both real and predicted motifs. As described above, per each of the enriched CLIP regions that were found by our analysis to harbor FBP binding site, we scanned for predicted FBPs throughout the entire span of the genomic feature in which the real CLIP FBP was reside in. Thus, by employing the database of predicted FAM binding sites we matched per each of the ±25 bp “Real FAM” regions an equivalent number of “Predicted FAM” regions (±25 bp) that proved to harbor icSHAPE signal within the 50 bp detection window. By employing these analytic criteria, we contrasted icSHAPE profile of “Real FBPs” cohort against icSHAPE profile of equal-size “Predicted FBPs” cohort (FIG. 7).

To further determine the contribution of co-clustering of FAMs within the same dCLIP transcript we split CLIP fragments into two batches, namely: Single FAMs per CLIP fiber (FAMs with zero adjacent FAMs on the same CLIP fiber), and Multiple FAMs per CLIP fiber (FAMs with one or more adjacent FAMs on the same CLIP fiber), and performed the distribution analysis of icSHAPE reactivity per each of these batches as depicted in FIG. 14.

The Denaturing CLIP (dCLIP) Methodology

Our original goal was to identify RNA interactomes for both canonical and non-canonical PRC1. We therefore initially used both CBX7 (canonical) and RYBP (non-canonical) as bait using conventional CLIP methodologies and CBX7-specific or RYBP-specific antibodies for the pulldown. However, all initial attempts failed due to high background, as evidenced by multiple bands that span the length of SDS PAGE gel (transferred to a CLIP membrane; FIG. 8A). Increasing washing stringency to 1 M NaCl and tagging CBX7 and RYBP with hemagglutinin (HA), for which strong antibodies have been developed over the years, did not significantly improve the outcome (FIG. 8B). The high background precluded efforts to purify specific bands corresponding to CBX7- and RYBP-RNA interactions. We concluded that higher stringency washes were necessary. To enable purification under maximal stringency, we took advantage of an existing in vivo biotin tagging system (Kim et al., 2009) and adapted the components to develop a new CLIP method that would enable purification under denaturing conditions. Indeed, biotin-streptavidin interactions have among the highest affinities and greatest specificity of any non-covalent biological interactions (Kd=10⁻¹⁵M), contrasting with K_{d}'s of 10⁻⁸-10⁻⁹ M for antigen-antibody interactions.

We introduced bio-tagged CBX7 and RYBP into ES cells stably expressing BirA biotinylase and performed “denaturing CLIP” or “dCLIP” with these features (FIG. 1A): (i) A biotin-tagged protein, (ii) in vivo UV-crosslinking to identify physiologically relevant interactions, (iii) an “RNAse protection step” to preserve the “footprint” in the RNA and trim away exposed RNA regions, (iv) a stringent denaturing purification method using streptavidin beads in the presence of 8M urea, 2% SDS, and 1M NaCl in order to eliminate RNA interactions not covalently photo-crosslinked to the protein of interest, (v) size selection in a denaturing SDS-PAGE gel with membrane transfer, and (vi) preparation of deep sequencing libraries after RNA extraction from membranes. The resulting pulldowns showed dramatically improved specificity, as evidenced by predominance of a single band for CBX7 and RYBP on the CLIP membranes and corresponding Western blots (FIG. 1B, FIG. 9A). CBX7 yielded a stronger band relative to RYBP on multiple biological replicates (FIG. 9B), potentially reflecting the evolutionary impact on RYBP's RNA binding domain (Tavares et al., 2012). Visualization of the RNA eluted from the CLIP membrane confirmed the presence of a population of CBX7-binding RNAs of heterogeneous size, reflecting the different degrees of RNase digestion (FIG. 9C). The eluted RNA was sensitive to RNase but not DNase treatment (FIG. 9C), consistent with specific elution of interacting transcripts, rather than chromatin.

Because of highly stringent denaturing conditions made possible with dCLIP, we asked if it were possible to skip the SDS-PAGE and membrane transfer steps entirely, as these steps partially served to eliminate RNA-protein interactions sensitive to denaturing SDS conditions as well. Furthermore, in principle, the exclusion of the additional steps could improve recovery of extremely limited quantities of RNA that are typically associated with epigenetic complexes. To test the possibility, we eluted RNAs directly from streptavidin beads using proteinase K treatment. However, we found that the purification by SDS-PAGE and membrane transfer was absolutely necessary in the dO ES cell samples, as direct elution from beads resulted in high background for some cellular samples (FIG. 10A). On the other hand, D7 ES cell samples fared better, with both elution methods demonstrated comparable enrichment of CBX7-binding RNAs as determined by specific sequencing tags density (FIG. 10B). On-bead elution was therefore more finicky and subject to cellular differences. We therefore prefer dCLIP performed in conjunction with SDS-PAGE purification followed by nitrocellulose membrane transfer to achieve the greatest specificity.

Elution of RYBP-interacting RNAs also produced a heterogeneous population, but lower levels of RNA were eluted overall (FIGS. 2A-C). Biotag-RYBP and -CBX7 were both expressed at physiological levels in independent clonal ES cells. For CBX7, we analyzed two clones (3E, 6F). The FPKM RNA-seq values were similar for control ES cells (FPKM=44.38) versus Biotag-CBX7-expressing cells (e.g., FPKM=46.03). Expression of Bio-tag-CBX7 also resulted in no major changes in the transcriptomic profile compared to control cells (FIG. 11A). Because of the strong enrichment of RNA for CBX7, our subsequent work focused strictly on CBX7-RNA interactions.

### Example 1

### dCLIP Defines RNA Footprints for CBX7

Peak-calling using PeakRanger Software (Feng et al., 2011) revealed 8,000-10,000 statistically significant peaks in three biological replicates (FIG. 1C, FIG. 12), among which these CBX7 binding peaks were concordant and reproducible, both at the level of comparing RefSeq transcript targets (FIG. 1D) and at the level of comparing genome-wide binding footprints (FIG. 11B). Only those appearing in at least two of three biological replicates were considered true positives. Concordant peaks mapped to 1,333 distinct transcripts, with many transcripts having multiple peaks/binding sites. Because the peaks corresponded to an RNase-protected fragment, each peak represented a CBX7 “footprint” or “binding site” in the associated RNA. The median binding site was 171 nt in length, with >90% of binding fragments falling in the range of 30-600 nt (FIG. 1E). Cis-regulatory element analysis (CEAS) indicated that a relatively small number of peaks (6.9%) mapped to intergenic transcripts (FIG. 13A). Intriguingly, although the BMI1 subunit of PRC1 was recently shown to be associated primarily with noncoding chromatin ((Ray et al., 2013; Ray et al., 2016), our CBX7 library was enriched for protein-coding messenger RNAs (mRNA). Indeed, >80% of peaks occurred within protein-coding transcripts, with the 3′ untranslated region (3′UTR) accounting for 56.7% of all dCLIP peaks and 64.6% of all peaks within coding transcripts (FIG. 13A,C). Consistent with this, metagene analysis showed major enrichment at the 3′ end of transcripts (FIG. 1F). Thus, the binding pattern for the canonical form of PRC1, as viewed through CBX7, is distinct from those of PRC2 and YY1, which tend to concentrate at the 5′ end of coding genes (Beltran et al., 2016; Kaneko et al., 2014a; Sigova et al., 2015; Zhao et al., 2010).

We compared the dCLIP tags to expression level of the respective RefSeq transcripts (input RNA-seq). Among transcripts without reproducible dCLIP binding, we identified a cohort of 2,078 transcripts that possessed similar expression levels as 1,333 transcripts with reproducible CLIP tags (green and black dots; FIG. 1G, 12), yet still lacked CBX7-binding footprints (red dots, FIG. 1G), thereby arguing against the CLIP profile being a random sampling of the ES transcriptome. Myl6, for example, was highly expressed, but no significant CBX7-binding sites were called within the transcript (FIG. 12). Conversely, the consistent enrichment of transcripts with low RNA-seq FPKM values provided strong evidence for specific enrichment of CBX7 dCLIP tags. Among the 1,333 CBX7 target transcripts, 135 were called “high binders”, due to highly enriched CLIP signals (FIG. 1G, green dots). For example, Dusp9 and Dcaf12l1 RNAs were highly enriched for CBX7 binding within its 3′UTR in three biological replicates, whereas the rest of the transcript was largely devoid of dCLIP tags (FIG. 1C). This pattern held for other CBX7-interacting transcripts, such as Calm2, for which the CBX7 binding sites were concordant within the 3′UTR among three biological replicates (FIG. 12). LncRNAs such as Tug1 were also targeted, but the binding pattern was distinct from that of coding RNAs: For lncRNAs, CBX7 interaction sites were typically observed all along the transcript, rather than concentrated at the 3′ end (FIG. 12). The reproducibility between biological replicates provides a first validation for our dCLIP methodology.

We next examined how CBX7-binding sites in the RNA (dCLIP-seq) relate to CBX7's chromatin binding sites (ChIP-seq). Previous work demonstrated that CBX7 tends to to bind large number of genomic loci in mouse ES cells {Morey, 2012 #1198}. Therefore, CBX7-RNA interactions identified by dCLIP method might theoretically arise from non-specific cross-linking between chromatin-bound CBX7 and RNAs transcribed in the vicinity. To rule out this possibility, we performed CBX7 ChIP-seq using the same ES cells (FIG. 13B,C). Among the 1,333 transcripts with CBX7 binding sites, only 12% were associated with a CBX7 ChIP peak in the same RefSeq locus (inclusive of promoter region) (FIG. 1C, blue tracks; FIG. 12, blue tracks; FIG. 13D, Table b).

TABLE b
Related to FIGS. 1E,F and FIG. 2. List of all genes for which CBX7 binds
both the transcript (dCLIP) and the locus (ChIP).
1110038B12Rik; Acaca; Acsl4; Adad1; Ado; Aebp2; Agrn; Akap11; Alg11; Amfr; Arf6;
Atp11a; Aurka; Bend4; Bmpr2; Bnc2; Calm2; Camk2b; Ccdc50; Ccng1; Cdc14b; Cfdp1;
Chsy1; Clstn1; Col4a1; Cpne3; Dag1; Dcaf12l1; Dcakd; Ddah1; Dennd1b; Dlg3; Dnmt3a;
Dpysl3; Dst; Dusp16; Dusp7; Dusp9; Egln1; Eif4e2; Esrrb; Exoc6b; Fam172a; Fam20b;
Fbxl5; Fstl1; Gab1; Git1; Gm13152; Gm37013; Grik3; Gtpbp1; Hmgn1; Hsd17b12;
Igf2bp3; Igf2r; Iqsec1; Ist1; Kars; Kcnq5; Kdm7a; Larp4; Lonrf1; Lrig1; Lrrc58; Macf1;
Man2a1; Mapk4; Mcl1; Med13l; Meg3; Mest; Mief1; Mllt10; Ndrg1; Nkain1; Nmnat2;
Nova2; Npcd; Nptx1; Nr6a1; Nudt4; Ostc; Pam; Pcdhga1; Pcdhga10; Pcdhga11; Pcdhga12;
Pcdhga2; Pcdhga3; Pcdhga4; Pcdhga5; Pcdhga6; Pcdhga7; Pcdhga8; Pcdhga9; Pcdhgb1;
Pcdhgb2; Pcdhgb4; Pcdhgb5; Pcdhgb6; Pcdhgb7; Pcdhgb8; Pcdhgc3; Pcdhgc4; Pcdhgc5;
Pde4d; Peg10; Plec; Plekha2; Podxl; Ppp2r2c; Prkaa2; Prkaca; Prkar2a; Prkd3; Ptbp3;
Pvrl1; Qk; Rac1; Rbm38; Reep1; Rere; Rimklb; Rnf187; Rsrc1; Scamp1; Sdc4; Sfmbt2;
Sfrp1; Sh3pxd2a; Slc30a1; Slc38a1; Smg1; Snd1; Snn; Socs7; Sord; Sox2ot; Spen; Spock2;
Spop; Ssh2; Stag2; Stox2; Stxbp5; Tbc1d16; Tfdp1; Tmem164; Tnfrsf21; Trim44;
Trp53inp2; Tub; Ugcg; Uggt1; Usp31; Vat1; Xist; Yes1; Zfp318; Zic2; Zmat3;

This percentage was significantly lower than that for bulk expressed transcripts in the ES cells (FIG. 13D). In fact, ChIP peaks did not generally overlap dCLIP peaks (FIG. 1C, F, 12, 13C). Whereas CBX7 dCLIP tags were enriched in the 3′UTRs of mRNAs, the CBX7 ChIP reads were enriched at the 5′ end around the transcription start site (TSS; FIG. 1F). Thus, the dCLIP profiles were strikingly different from ChIP-seq profiles arguing strongly in favor of specific CBX7-RNA interactions detected by dCLIP method.

### Example 2

### Consensus Motifs Deduced from RNA Footprints

With a median footprint of 171 nt, the short and reproducible binding sites for CBX7 raised the possibility of defining consensus motifs for CBX7-containing PRC1 complexes. To deduce consensus motifs in the RNA, we performed comparative sequence analysis of CBX7-binding peaks from three dCLIP biological replicates (FIG. 2). In brief, we developed a pipeline for de novo motif identification in which we independently searched each dCLIP library (Lib.1, 2, 3; FIG. 2A) for motifs within CBX7 peaks (see STAR Methods for details). The resulting motifs were clustered based on sequence similarity into 4 distinct Familial binding profiles associated modules (FAMs) (FIG. 2B).

If the deduced motifs represented a true CBX7 RNA-binding consensus, we should expect to see enrichment of the FAM motifs in the 3′UTR. Indeed, consistent with CEAS analysis of the dCLIP peaks (FIG. 1E, FIG. 13A,C), motifs in all four FAMs were enriched in the 3′UTR (FIG. 2C). However, not all consensus sites were necessarily occupied by CBX7 in ES cells, as determined by dCLIP (FIG. 2D). For instance, only 30% of FAM1-bearing sequences in 3′UTR regions bound CBX7. The FAM-occupancy ratio—defined as the ratio of consensus sites bound by CBX7 to all consensus sites—was typically <1.0 for the 3′UTR and introns and was, interestingly, higher for the 5′UTR and coding regions (CDs). Thus, presence of a single consensus motif was not deterministic for CBX7 binding to target transcript, as is often the case for other RNA binding proteins (Taliaferro et al., 2016; Van Nostrand et al., 2016). Additional parameters, such as presence of various protein factors, binding site accessibility, and/or other CBX7 FAM motifs, could all play a role in enabling CBX7 interactions with predicted binding sites.

Another consideration is that CBX7 could have multiple contact points within one transcript, potentially contacting different faces of the RNA via different motifs. To test the latter possibility, we asked whether the motifs have a tendency to congregate on the same CLIP fragment. Analysis of all pairwise combinations of the FAMs revealed that they co-clustered, creating motif-pairs separated by ≤50 nt (FIG. 3A,B). The FAM1-FAM1 motif pair was found to be the most prevalent pair, followed by the FAM1-FAM2 pair. Other FAM couplings were also found at relatively high frequencies (FIG. 3A,B). These findings indicate that CBX7 motifs have a tendency to cluster and make possible the idea that more than one family of binding motifs might be necessary to constitute a recognition site within a given CBX7-binding transcript. Indeed, a majority of dCLIP fibers (CBX7 footprints) contained more than one FAM (FIG. 3C). We compared the FAM occupancy ratios of fibers/footprints with one FAM versus those of fibers/footprints containing multiple FAMs (FIG. 3D). Interestingly, for the 3′UTR, CBX7 footprints harboring clustered motifs demonstrated higher FAM occupancy ratios than those harboring only a single motif. Thus, to CBX7 is more likely to bind to the 3′UTR in vivo when FAM motifs are clustered, hinting at the possibility of cooperative interactions between CBX7 and RNA.

Given that both 5′ and 3′ UTRs are typically bound by large number of proteins (Glisovic et al., 2008), we asked how the CBX7 motifs might be related to binding motifs of known RNA-binding proteins. A similarity matching analysis of the 4 FAMs against a panel of >1,000 known binding motifs uncovered significant overlap (FIG. 4A). For example, FAM1 showed significant similarity to the motif for PUMILIO, a family of proteins involved in RNA degradation and inhibition of RNA translation (Spassov and Jurecic, 2003). Also demonstrating significant similarities are motifs for the RNA splicing regulator, epithelial splicing regulatory protein 1 (ESRP1; also known as RBM35a) (Warzecha et al., 2009); the cytoplasmic poly(A) binding protein (PABPC), which mediates ribosome recruitment and translation initiation of target transcripts (Bag and Bhattacharjee, 2010)); and the serine/arginine-rich splicing factor 1 (SRSF1), another regulator of RNA splicing. Thus, CBX7 motifs appear to possess prominent characteristics that overlap those of known RNA binding proteins.

We also asked whether the binding sites possess structural features by taking advantage of structural profiles established in mouse ES cells via click selective 2′-hydroxyl acetylation and profiling experiments (icSHAPE) (Spitale et al., 2015). icSHAPE-seq allows probing of RNA secondary structure both in vivo and in vitro and favors single-stranded or flexible RNA regions. icSHAPE-seq also offers advantages over DMS-seq and Cirs-seq (Incarnato et al., 2014; Rouskin et al., 2014), as it is reactive to all four nucleotides, thereby enabling the capture of RNA secondary structures at a transcriptome-wide level at higher resolution (Spitale et al., 2015). For each of the four FAMs, icSHAPE profiles were markedly different from one another (FIG. 4B), suggesting that each FAM possesses a unique RNA secondary structure. In proximity to their center, FAM1 and FAM4 had a clear preference for more unfolded structures, whereas FAM2 preferred a folded configuration. Notably, albeit being similar by pattern, the average icSHAPE profiles of all real FAMs were found to have a higher icSHAPE signal than those of their control counterparts. Because icSHAPE signals positively correlate with unfolded (more open or reactive) conformations, greater icSHAPE signals over real FAMs in comparison to their control counterparts suggest that CBX7 gravitates towards RNAs with an unfolded conformation. Similar icSHAPE profiles were observed both in vivo versus in vitro, arguing against marked interference from RNA binding proteins in vivo.

We then repeated the analysis for dCLIP fibers with clustered FAM motifs (FIG. 14). In the case of FAM1 and FAM4 in vivo, the clustering on the dCLIP fibers correlated with higher icSHAPE reactivity in comparison to dCLIP fibers with a single FAM. FAM clustering might therefore predispose to an open conformation in vivo. Differences between in vivo and in vitro profiles (FIG. 14) likely reflect inherent folding differences dependent on multiplicity of FAMs, in addition to cellular factors/binding-proteins that are only available in vivo. Collectively, our data support the idea of CBX7-binding sites being embedded in the conformationally accessible regions of associated RNAs, of secondary structures that may be governed by linear sequence motifs, and of congregated motifs that facilitate in vivo binding of CBX7 to RNA.

### Example 3

### Biochemical Validation of CBX7-Binding Sites

Next we turned to experimental systems to validate and understand the nature of the CBX7-3′UTR interactions. First, we sought to confirm select interactions using a different method of in vivo RNA pulldown and using antibodies to a different epitope of the tagged protein (as opposed to using the biotin tag to pull down CBX7). Native RIP with qPCR confirmed the enrichment of Dusp9, Calm2, and Tug1 RNAs in multiple independent biological replicates (FIG. 5A). The negative control U1 RNA was not enriched in spite of the high abundance of this RNA used for splicing.

Second, to confirm direct interactions between CBX7 and various 3′UTRs, we performed RNA electrophoretic mobility shift assays (EMSA) using CBX7 protein purified from baculovirus and purified in vitro transcribed RNAs corresponding to dCLIP peaks. We tested three representative transcripts, Calm2, Dusp9, and Dcaf12l1 (FIG. 1C, FIG. 12). RNA probes were generated from the 3′UTR binding sites Dcaf12l1, with Calm2 harboring clustered FAM1+FAM2 motifs, Dusp9 harboring clustered FAM3+FAM4 motifs, and Dcaf12l1 harboring only FAM4 motifs. Consistent with icSHAPE and the potential for secondary structures, native gels for purified unbound Dcaf12l1, Calm2, and Dusp9 probes yielded multiple bands (green arrows, FIG. 5B), suggesting conformationally complex RNAs. Addition of CBX7 protein resulted in mobility shifts for all three RNAs (asterisk, FIG. 5B), whereas addition of a control protein GFP did not. The shift for Dcaf12l1 was especially robust (red arrow, FIG. 5B, left lanes; FIG. 5C), and the interaction was competed away by excess cold Dcaf12l1 oligos (FIG. 5D). CBX7's dissociation constants (Kd) for Dcaf12l1, Calm2, and Dusp9 3′UTRs suggested affinities in the low micromolar range (FIG. 5E), consistent with previous assessments of CBX7-RNA interactions (Bernstein et al., 2006; Yap et al., 2010). Interestingly, however, its Hill coefficients suggested the potential for positive cooperativity at 2-3 binding sites per fragment in each CBX7-3′UTR interaction in vitro (FIG. 5E). Positive cooperativity binding mode was concurrent with the gradual increase in distance between shifted and non-shifted fragments following increase in CBX7 concentration (FIG. 5C)—pattern consistent with cooperative binding mode (Wang and Bell, 1994). Notably, our FAM motif analysis suggested that co-clustering is also correlated with higher FAM occupancy rates in vivo (FIG. 3D). Thus, while the overall binding affinity of CBX7 is relatively low (Kd in the micromolar range), the potential for positive cooperativity may considerably change the dynamics in the cellular setting.

We next tested the relevance of the bioinformatically predicted FAM motifs. We turned to footprints with single FAMs in order to simplify the analysis. For the FAM3 motif in the 3′UTR of Nucks1 mRNA, CBX7 shifted the RNA fragment and the shift was reduced by Nucks1 cold competitors (FIG. 5F,G). The shift was weaker for the single motif than for the 3′UTRs of Dcaf12l1, Calm2, and Dusp9, each of which contained multiple motifs—again consistent with the idea of positive cooperativity in CBX7-RNA interactions. Nevertheless, mutating the FAM3 site reduced CBX7 binding. Similar results were obtained for a single FAM1 site in the 3′UTR of Larp1 mRNA (FIG. 5F, right lanes). Taken together these data demonstrate that CBX7 directly binds the 3′UTR domains identified by dCLIP, thereby validating dCLIP as one method of identifying RNA footprints and consensus motifs.

### Example 4

### Targeting CBX7-Binding Sites In Vivo Results in Gene Upregulation

We explored potential functions of the CBX7-3′UTR interactions. Given that PRC1 is generally involved in gene repression (Simon and Kingston, 2013), we asked whether the RNA-binding activity of CBX7 may be involved in recruiting PRC1 to silence genes. To test this idea, we attempted to block the CBX7-3′UTR interactions and designed antisense oligonucleotides (ASO) comprising interspersed DNA bases and locked nucleic acids (LNA) bases to create “LNA mixmers” that are not subject to RNaseH-mediated target degradation and can therefore stably associate with target sequences (Sarma et al., 2010). For each transcript, we designed a pool of LNA mixmers to the corresponding 3′UTR peaks (FIG. 1C, FIG. 12, orange boxes). We administered the pooled LNAs to ES cells and measured effects on gene-specific expression after 24 hours. Intriguingly, targeting the CBX7-binding sites in the 3′UTR of Calm2 and Dcaf12l1 transcripts resulted in a significant 3.67- and 2.68-fold upregulation of both transcripts, respectively (FIG. 6A). Because Calm2 and Dcaf12l1 were already highly expressed transcripts in ES cells, this degree of upregulation was substantial. The upregulation was gene-specific, as Calm2 LNAs had no effect on either Dcaf12l1 or Dusp9 expression, and Dcaf12l1 LNAs had no significant effect on Calm2 or Dusp9 expression. Moreover, a negative control LNA also resulted in no changes in gene expression. These data demonstrate that gene-specific LNAs directed at the CBX7-3′UTR interactions lead to a specific mRNA upregulation of the target gene.

The repressive activity of PRC1 has been linked to both the H2AK119 ubiquitylation function and to chromatin compaction (Simon and Kingston, 2013). To understand how LNA treatment enhanced gene activity, we performed ChIP-qPCR to ask whether there were locus-specific changes to CBX7 recruitment and H2AK119Ub. Interestingly, we observed no changes in CBX7 recruitment and H2AK119 ubiquitylation at either Calm2 or Dcaf12l1 after treatment with corresponding gene-specific LNAs (FIG. 6B). To determine whether chromatin compaction was affected, we performed Formaldehyde-assisted Isolation of Regulatory Elements (FAIRE) analysis (Giresi et al., 2007) but also found no evident differences in chromatin accessibility when measured at two sites, one corresponding to a DHS and the other to a DNaseI-resistant site (FIG. 1C, FIG. 12), within Calm2 and Dcaf12l1 (FIG. 6C). This is consistent with studies indicating that CBX7 complexes do not play a role in nucleosome compaction (Grau et al., 2011). These data suggest that the upregulation observed after LNA treatment is not a consequence of chromatin changes relating to either PRC1's chromatin compaction function or its H2AK119Ub function. LNA-mediated gene upregulation could result from either co-transcriptional (e.g., elongation, splicing) or post-transcriptional mechanisms (e.g., RNA processing, stabilization). To test this idea, we examined changes in the levels of nascent transcripts (pre-mRNA) by performing RT-qPCR using intronic primer pairs. For Calm2, nascent transcript levels did not change upon treatment with Calm2-specific LNAs, but Calm2 processed mRNA levels increased (FIG. 6D), consistent with the idea of a post-transcriptional mechanism, such as RNA stabilization. For Dcaf12l1, both nascent and processed RNA levels increased (FIG. 6D), suggesting that there could be contributions from co-transcriptional and/or post-transcriptional mechanisms.

Next, we examined the effect of LNA oligomers on CBX7 binding to target RNAs in vitro. Intriguingly, while RNA EMSA showed that pre-incubating RNA with gene-specific LNAs resulted in an upward shift of the transcripts, as expected (blue arrows, FIG. 5B), the LNA did not block or displace CBX7 binding. Rather, it supershifted the CBX7-RNA complex and substantially enhanced CBX7 binding to RNA (red arrowheads, FIG. 5B). This CBX7-3′UTR supershift occurred only when incubated with the gene-specific LNA and not with control LNA. This specificity was observed in all three cases (Dcaf12l1, Calm2, and Dusp9). Thus, LNA binding appears to stabilize CBX7 interaction with the 3′UTR, producing much more robust gel shifts between CBX7 and the 3′UTR motifs in the presence of the LNAs in vitro.

To determine whether the LNA-mediated gene upregulation depended on CBX7 in vivo, we introduced the LNAs into wildtype versus Cbx7−/− ES cells (Cheng et al., 2014; Zhen et al., 2016) (FIG. 6, 15). Using Dcaf12l1 as the test case, we observed that upregulation of nascent and processed RNA by gene-specific LNAs occurred only in the presence of CBX7, and was significantly blunted when CBX7 was deleted (FIG. 6E). No effects were seen with negative control LNAs (LNA-Calm2, LNA-CtrlA). Thus, LNA-mediated gene upregulation is indeed a CBX7-dependent process. It is known that CBX8 is upregulated in ES cells when CBX7 is depleted, in order to maintain stem cell self-renewal (Morey et al., 2012; O'Loghlen et al., 2012) (FIG. 15B). The functional compensation by CBX8 is consistent with the lack of Dcaf12l1 downregulation in Cbx7−/− cells. Interestingly, however, the gene upregulation effect by the LNA was specific to CBX7. Taken together, these data demonstrate that the LNA-mediated gene upregulation is a CBX7-dependent process (FIG. 6E, 15), most likely involving both co-transcriptional and post-transcriptional mechanisms (FIG. 6D). It may also involve enhanced binding of CBX7 to the 3′UTR (FIG. 6B). Thus, CBX7—when bound to the 3′UTR—may paradoxically enhance expression of the target transcript. Consistent with this idea, analysis of probability density function revealed that transcripts bound by CBX7 (dCLIP) have a higher likelihood of expression (FPKM) than transcripts not targeted by CBX7 (FIG. 6F).

The localization of CBX7 to the 3′UTR (FIG. 1, 12) in close proximity to motifs for regulators of transcript stability (PUM) and nuclear-cytoplasmic RNA localization (PolyA-binding protein (PABPC)) (FIG. 4A) might suggest a post-transcriptional component in the LNA-mediated gene upregulation. To determine whether there was a concomitant increase at the protein level, we developed a quantitative Western blot analysis for DCAF12L1 protein and measured protein upregulation in the linear range of the assay (FIG. 6G). When Dcaf12l1-specific LNAs were administered to ES cells, we observed a 50-100% upregulation of DCAF12L1 protein in multiple biological replicates (FIG. 6H,I). Thus, although no chromatin changes were evident, the increase in mRNA levels was mirrored by an increase in protein expression. These observations are consistent with a co-transcriptional and/or post-transcriptional mechanism of gene regulation by CBX7, with enhancement of upregulation following administration LNAs targeting FAM motifs.

### Example 5

### dCLIP Analysis of Human CBX7 (hCBX7) Identifies Shared Consensus Motifs

Next, we applied dCLIP methodology to human CBX7 protein to assess whether the human orthologue shares RNA binding potential and to determine whether consensus motifs can be independently deduced from the human RNA-protein interactions. Although hCBX7 and mouse CBX7 (mCBX7) share CD and PC boxes, hCBX7 is 58 amino acids longer than mCBX7 and is therefore epitopically different (FIG. 16A,B). Nevertheless, the dCLIP methodology could be applied because the biotag rendered the baits equivalent. We performed dCLIP in human embryonic kidney cells (HEK293) and followed the same analysis pipeline developed for mCBX7 (FIG. 2A). hCBX7 indeed also bound a family of RNAs. We identified 4,772 binding peaks total, corresponding to 3,729 RefSeq transcripts. The average hCBX7 footprint size was 183 nt (FIG. 7A). CEAS analysis showed that hCBX7 also preferentially bound 3′UTRs of mRNAs (FIG. 7B, 16C,D). The representative gene, IRAK1, illustrated the 3′ preference of hCBX7 for mRNA (FIG. 7C). Because the hCBX7 footprints were also small (FIG. 7A), application of our bioinformatic pipeline enabled deduction of 9 families of consensus motifs (FIG. 7D). Intriguingly, out of 9 hCBX7 FBPs, 4 FBPs co-clustered with (bore similarity to) mouse FBPs, whereas 5 FBPs were hCBX7-specific. We confirmed select transcripts for binding to hCBX7 by UV-RIP-qPCR and observed concordant results (FIG. 7E). As was the case for mCBX7 (FIG. 4A), enriched RNA motifs for hCBX7 shared similarities with motifs for PUMILIO, and SRSF1 (FIG. 7F). Thus, hCBX7 and mCBX7 share consensus motifs for RNA binding. Notably, these motifs were independently deduced by separate dCLIP and bioinformatic analyses. Nonetheless, 5 FBPs were hCBX7-specific, consistent with its having an extra 58 amino acids that could in principle confer additional binding activities.

Finally, we examined the relationship between mCBX7/hCBX7 transcripts as defined by dCLIP and BMI1 transcripts as defined by gradient RNA immunoprecipitation (GRIP) in human HeLa cells (Ray et al., 2016). GRIP method involves formaldehyde cross-linking and gradient purification of chromatin fraction with subsequent immunoprecipitation of chromatin-bound RNAs using antibodies against the BMI1 subunit of PRC1. Despite substantial differences in methodology, there was considerable overlap, with 1,777 transcripts shared between hCBX7 and hBMI1 (Table C). This represented to nearly half of hCBX7-interacting transcripts—the 3′UTR of IRAK1 being one example (FIG. 7C). Taken together, these data validate the dCLIP methodology and pipeline and provide proof-of-concept that the technique can be applied to different epitopes in different species.

TABLE c
Related to FIG. 7. List of genes that produced specific dCLIP CBX7
binding in human HEK293 cells and GRIP BMI1 binding in human
HeLa cells (GRIP data adopted from (Ray et al., 2016).

| Refseq | Ensembl | Gene Name |
|---|---|---|
| NM_005885 | ENSG00000145495 | membrane associated ring finger 6 |
| NM_006640 | ENSG00000184640 | septin 9 |
| NM_004996 | ENSG00000103222 | ABCC1 |
| NM_001171 | ENSG00000091262 | ABCC6 |
| NM_018358 | ENSG00000161204 | ABCF3 |
| NM_022437 | ENSG00000143921 | ABCG8 |
| NM_198147 | ENSG00000168792 | ABHD15 |
| NM_021214 | ENSG00000136379 | ABHD17C |
| NM_025097 | ENSG00000164074 | ABHD18 |
| NM_005470 | ENSG00000136754 | ABIl |
| NM_005157 | ENSG00000097007 | ABL1 |
| NM_005158 | ENSG00000143322 | ABL2 |
| NM_002313 | ENSG00000099204 | ABLIM1 |
| NM_001092 | ENSG00000159842 | ABR |
| NM_145804 | ENSG00000166016 | ABTB2 |
| NM_001093 | ENSG00000076555 | ACACB |
| NM_000019 | ENSG00000075239 | ACAT1 |
| NM_022735 | ENSG00000182827 | ACBD3 |
| NM_014977 | ENSG00000100813 | ACIN1 |
| NM_004457 | | ACSL3 |
| NM_001101 | ENSG00000075624 | ACTB |
| NM_024855 | ENSG00000101442 | ACTR5 |
| NM_006988 | ENSG00000154734 | ADAMTS1 |
| NM_012091 | | ADAT1 |
| NM_182503 | ENSG00000189007 | ADAT2 |
| NM_020247 | ENSG00000163050 | ADCK3 |
| NR_110007 | ENSG00000259456 | ADNP-AS1 |
| NR_040107 | ENSG00000260898 | ADPGK-AS1 |
| NM_032550 | ENSG00000169129 | AFAP1L2 |
| NM_005935 | ENSG00000172493 | AFF1 |
| NM_012154 | ENSG00000123908 | AGO2 |
| NM_017629 | ENSG00000134698 | AGO4 |
| NM_020132 | ENSG00000160216 | AGPAT3 |
| NM_024929 | ENSG00000279355 | AGPAT4-IT1 |
| NM_015239 | ENSG00000135049 | AGTPBP1 |
| NM_015328 | ENSG00000158467 | AHCYL2 |
| NM_017651 | ENSG00000135541 | AHI1 |
| NM_005858 | ENSG00000105127 | AKAP8 |
| NM_014371 | ENSG00000011243 | AKAP8L |
| NM_024595 | ENSG00000174574 | AKIRIN1 |
| NR_002796 | | AKR7A2P1 |
| NM_000034 | ENSG00000149925 | ALDOA |
| NM_006982 | ENSG00000180318 | ALX1 |
| NM_000479 | ENSG00000104899 | AMH |
| NM_030943 | ENSG00000166126 | AMN |
| NM_016238 | ENSG00000196510 | ANAPC7 |
| NM_000037 | ENSG00000029534 | ANK1 |
| NM_015114 | ENSG00000176915 | ANKLE2 |
| NM_032217 | ENSG00000132466 | ANKRD17 |
| NM_144994 | ENSG00000163126 | ANKRD23 |
| NM_014915 | ENSG00000107890 | ANKRD26 |
| NM_015199 | ENSG00000206560 | ANKRD28 |
| NR_026844 | ENSG00000214262 | ANKRD36BP1 |
| NM_016466 | ENSG00000213337 | ANKRD39 |
| NM_152326 | ENSG00000156381 | ANKRD9 |
| NM_006401 | ENSG00000136938 | ANP32B |
| NM_030920 | ENSG00000143401 | ANP32E |
| NM_004039 | ENSG00000182718 | ANXA2 |
| NM_001153 | ENSG00000196975 | ANXA4 |
| NM_001154 | ENSG00000164111 | ANXA5 |
| NM_001158 | ENSG00000131480 | AOC2 |
| NM_004068 | ENSG00000161203 | AP2M1 |
| NM_003664 | ENSG00000132842 | AP3B1 |
| NM_001163 | ENSG00000107282 | APBA1 |
| NM_006051 | ENSG00000113108 | APBB3 |
| NM_030642 | ENSG00000128313 | APOL5 |
| NM_000484 | ENSG00000142192 | APP |
| NM_015242 | ENSG00000186635 | ARAP1 |
| NM_001658 | ENSG00000143761 | ARF1 |
| NM_004308 | ENSG00000175220 | ARHGAP1 |
| NM_021226 | ENSG00000128805 | ARHGAP22 |
| NR_046816 | ENSG00000230789 | ARHGAP26-IT1 |
| NM_004309 | ENSG00000141522 | ARHGDIA |
| NM_033415 | ENSG00000105676 | ARMC6 |
| NM_003976 | ENSG00000117407 | ARTN |
| NM_139058 | ENSG00000004848 | ARX |
| NM_019893 | ENSG00000188611 | ASAH2 |
| NR_002765 | | ASAP1-IT1 |
| NM_017873 | ENSG00000148331 | ASB6 |
| NM_001672 | ENSG00000101440 | ASIP |
| NM_004318 | ENSG00000198363 | ASPH |
| NM_015338 | ENSG00000171456 | ASXL1 |
| NM_032810 | ENSG00000138138 | ATAD1 |
| NM_007041 | ENSG00000107669 | ATE1 |
| NM_005171 | ENSG00000123268 | ATF1 |
| NM_018179 | ENSG00000171681 | ATF7IP |
| NM_033388 | ENSG00000168010 | ATG16L2 |
| NM_006395 | ENSG00000197548 | ATG7 |
| NM_001940 | ENSG00000111676 | ATN1 |
| NM_024524 | ENSG00000133657 | ATP13A3 |
| NM_000701 | ENSG00000163399 | ATP1A1 |
| NM_032766 | ENSG00000203865 | ATP1A1-AS1 |
| NM_001681 | ENSG00000174437 | ATP2A2 |
| NM_001682 | ENSG00000070961 | ATP2B1 |
| NM_014382 | ENSG00000017260 | ATP2C1 |
| NM_000705 | ENSG00000186009 | ATP4B |
| NM_001686 | ENSG00000110955 | ATP5B |
| NM_001688 | | ATP5F1 |
| NM_001685 | ENSG00000154723 | ATP5J |
| NM_004889 | ENSG00000241468 | ATP5J2 |
| NM_001697 | ENSG00000241837 | ATP5O |
| NM_001694 | ENSG00000185883 | ATP6V0C |
| NM_003945 | | ATP6V0E1 |
| NM_000489 | ENSG00000085224 | ATRX |
| NM_001698 | ENSG00000148090 | AUH |
| NM_015060 | ENSG00000105778 | AVL9 |
| NM_021732 | ENSG00000119986 | AVPI1 |
| NM_003502 | ENSG00000103126 | AXIN1 |
| NM_152490 | ENSG00000162885 | B3GALNT2 |
| NM_012200 | ENSG00000149541 | B3GAT3 |
| NM_004776 | ENSG00000158470 | B4GALT5 |
| NM_020064 | ENSG00000125492 | BARHL1 |
| NM_023005 | ENSG00000009954 | BAZ1B |
| NM_013449 | ENSG00000076108 | BAZ2A |
| NM_014567 | ENSG00000050820 | BCAR1 |
| NM_003567 | ENSG00000137936 | BCAR3 |
| NM_000633 | | BCL2 |
| NM_014739 | ENSG00000029363 | BCLAF1 |
| NM_004327 | ENSG00000186716 | BCR |
| NM_004459 | ENSG00000171634 | BPTF |
| NM_004333 | ENSG00000157764 | BRAF |
| NM_014577 | ENSG00000100425 | BRD1 |
| NM_023924 | ENSG00000028310 | BRD9 |
| NM_032043 | ENSG00000136492 | BRIP1 |
| NM_153252 | ENSG00000165288 | BRWD3 |
| NM_014962 | ENSG00000132640 | BTBD3 |
| NM_001207 | ENSG00000145741 | BTF3 |
| NM_003939 | ENSG00000166167 | BTRC |
| NM_004725 | ENSG00000154473 | BUB3 |
| NM_032024 | ENSG00000148655 | C10orf11 |
| NM_024541 | ENSG00000120029 | C10orf76 |
| NM_170746 | ENSG00000211450 | C11orf31 |
| NM_004894 | ENSG00000156411 | C14orf2 |
| NM_032366 | ENSG00000130731 | C16orf13 |
| NM_025108 | ENSG00000162062 | C16orf59 |
| NM_181655 | ENSG00000186665 | C17orf58 |
| NM_001085430 | ENSG00000214226 | C17orf67 |
| NM_031446 | ENSG00000141428 | C18orf21 |
| NM_024038 | ENSG00000123144 | C19orf43 |
| NM_178830 | ENSG00000160392 | C19orf47 |
| NM_138358 | ENSG00000142444 | C19orf52 |
| NM_001025495 | ENSG00000162913 | C1orf145 |
| NM_017891 | ENSG00000131591 | C1orf159 |
| NM_001010979 | | C1orf189 |
| NM_001212 | ENSG00000108561 | C1QBP |
| NM_030945 | | C1QTNF3 |
| NM_001014442 | | C1QTNF9B-AS1 |
| NM_080828 | ENSG00000125975 | C20orf173 |
| NM_018840 | ENSG00000101084 | C20orf24 |
| NM_017874 | ENSG00000101220 | C20orf27 |
| NM_058180 | ENSG00000160298 | C21orf58 |
| NM_032561 | ENSG00000128346 | C22orf23 |
| NM_017880 | ENSG00000115998 | C2orf42 |
| NM_173649 | ENSG00000239605 | C2orf61 |
| NM_023073 | ENSG00000197603 | C5orf42 |
| NM_001277348 | | C5orf66 |
| NM_178508 | ENSG00000186577 | C6orf1 |
| NM_001029863 | | C6orf120 |
| NM_030939 | | C6orf62 |
| NM_001130929 | ENSG00000243317 | C7orf73 |
| NM_001080482 | | C9orf172 |
| NM_018956 | ENSG00000165698 | C9orf9 |
| NM_138375 | ENSG00000134508 | CABLES1 |
| NM_020898 | ENSG00000012822 | CALCOCO1 |
| NM_004342 | ENSG00000122786 | CALD1 |
| NM_001743 | ENSG00000143933 | CALM2 |
| NM_005184 | ENSG00000160014 | CALM3 |
| NM_033429 | ENSG00000129007 | CALML4 |
| NM_001745 | ENSG00000164615 | CAMLG |
| NM_015447 | ENSG00000130559 | CAMSAP1 |
| NM_015215 | ENSG00000171735 | CAMTA1 |
| NM_018448 | | CAND1 |
| NM_001746 | ENSG00000127022 | CANX |
| NM_000070 | ENSG00000092529 | CAPN3 |
| NM_004291 | ENSG00000164326 | CARTPT |
| NM_020764 | ENSG00000167971 | CASKIN1 |
| NR_132322 | ENST00000428155 | CASP16P |
| NM_005189 | ENSG00000173894 | CBX2 |
| NM_014292 | ENSG00000183741 | CBX6 |
| NM_145045 | ENSG00000198003 | CCDC151 |
| NR_034089 | | CCDC18-AS1 |
| NM_001282544 | ENSG00000166329 | CCDC182 |
| NM_005436 | ENSG00000108091 | CCDC6 |
| NM_001144995 | | CCDC85C |
| NM_018318 | ENSG00000123106 | CCDC91 |
| NM_052848 | ENSG00000142039 | CCDC97 |
| NM_001243212 | ENSG00000262484 | CCER2 |
| NM_005190 | ENSG00000112237 | CCNC |
| NM_053056 | ENSG00000110092 | CCND1 |
| NM_006835 | ENSG00000118816 | CCNI |
| NM_003858 | | CCNK |
| NM_030937 | ENSG00000221978 | CCNL2 |
| NM_145012 | ENSG00000108100 | CCNY |
| NM_006430 | ENSG00000115484 | CCT4 |
| NM_012073 | ENSG00000150753 | CCT5 |
| NM_006429 | ENSG00000135624 | CCT7 |
| NM_006016 | ENSG00000135535 | CD164 |
| NM_139286 | ENSG00000176386 | CDC26 |
| NM_020240 | ENSG00000158985 | CDC42SE2 |
| NM_080668 | ENSG00000146670 | CDCA5 |
| NM_022124 | ENSG00000107736 | CDH23 |
| NM_006201 | ENSG00000102225 | CDK16 |
| NM_004642 | ENSG00000111328 | CDK2AP1 |
| NM_016082 | ENSG00000101391 | CDK5RAP1 |
| NM_001261 | ENSG00000136807 | CDK9 |
| NM_017774 | ENSG00000145996 | CDKAL1 |
| NM_003948 | | CDKL2 |
| NM_003818 | ENSG00000101290 | CDS2 |
| NM_004824 | ENSG00000153046 | CDYL |
| NM_005195 | | CEBPD |
| NM_001806 | ENSG00000153879 | CEBPG |
| NM_006560 | ENSG00000149187 | CELF1 |
| NM_018455 | ENSG00000166451 | CENPN |
| NM_024322 | ENSG00000138092 | CENPO |
| NM_018140 | ENSG00000112877 | CEP72 |
| NM_013384 | ENSG00000143418 | CERS2 |
| NM_013242 | ENSG00000070761 | CFAP20 |
| NM_005507 | ENSG00000172757 | CFL1 |
| NM_024111 | ENSG00000128965 | CHAC1 |
| NM_001273 | ENSG00000111642 | CHD4 |
| NM_015557 | ENSG00000116254 | CHD5 |
| NM_020920 | ENSG00000100888 | CHD8 |
| NM_001275 | ENSG00000100604 | CHGA |
| NM_000390 | ENSG00000188419 | CHM |
| NM_024591 | ENSG00000176108 | CHMP6 |
| NM_152272 | ENSG00000147457 | CHMP7 |
| NM_017444 | ENSG00000104472 | CHRAC1 |
| NM_012125 | | CHRM5 |
| NM_020402 | ENSG00000129749 | CHRNA10 |
| NM_000748 | ENSG00000160716 | CHRNB2 |
| NM_004273 | ENSG00000122863 | CHST3 |
| NM_004804 | | CIAO1 |
| NM_006384 | ENSG00000185043 | CIB1 |
| NM_015125 | ENSG00000079432 | CIC |
| NM_152480 | | CIRBP-AS1 |
| NM_004143 | ENSG00000125931 | CITED1 |
| NM_006825 | ENSG00000136026 | CKAP4 |
| NM_001827 | ENSG00000123975 | CKS2 |
| NM_015282 | ENSG00000074054 | CLASP1 |
| NM_015097 | ENSG00000163539 | CLASP2 |
| NM_005602 | | CLDN11 |
| NM_014343 | ENSG00000106404 | CLDN15 |
| NM_001111319 | ENSG00000177300 | CLDN22 |
| NM_015226 | ENSG00000038532 | CLEC16A |
| NM_001080511 | ENSG00000236279 | CLEC2L |
| NM_014666 | ENSG00000113282 | CLINT1 |
| NM_001291 | ENSG00000176444 | CLK2 |
| NM_024769 | | CLMP |
| NM_018941 | ENSG00000182372 | CLN8 |
| NM_001833 | ENSG00000122705 | CLTA |
| NM_001835 | ENSG00000070371 | CLTCL1 |
| NM_144601 | ENSG00000140931 | CMTM3 |
| NM_182553 | ENSG00000174871 | CNIH2 |
| NM_016284 | ENSG00000125107 | CNOT1 |
| NM_014515 | ENSG00000111596 | CNOT2 |
| NM_018224 | ENSG00000106603 | COA1 |
| NM_001008215 | ENSG00000183513 | COA5 |
| NM_015198 | ENSG00000106078 | COBL |
| NM_153603 | ENSG00000168434 | COG7 |
| NM_032518 | ENSG00000188517 | COL25A1 |
| NM_000495 | ENSG00000188153 | COL4A5 |
| NM_024656 | ENSG00000130309 | COLGALT1 |
| NM_000754 | ENSG00000093010 | COMT |
| NM_016128 | ENSG00000181789 | COPG1 |
| NM_144576 | ENSG00000135469 | COQ10A |
| NM_001302 | ENSG00000241563 | CORT |
| NM_016468 | ENSG00000133983 | COX16 |
| NM_001865 | ENSG00000112695 | COX7A2 |
| NM_014912 | ENSG00000107864 | CPEB3 |
| NM_003915 | ENSG00000214078 | CPNE1 |
| NR_002763 | ENSG00000280837 | CPS1-IT1 |
| NM_006693 | ENSG00000160917 | CPSF4 |
| NM_004380 | ENSG00000005339 | CREBBP |
| NM_021212 | | CREBZF |
| NM_016441 | ENSG00000150938 | CRIM1 |
| NM_001312 | ENSG00000182809 | CRIP2 |
| NM_015986 | ENSG00000176390 | CRLF3 |
| NM_006371 | ENSG00000170275 | CRTAP |
| NM_001316 | ENSG00000124207 | CSE1L |
| NR_027320 | | CSNK1A1P1 |
| NM_001893 | ENSG00000141551 | CSNK1D |
| NM_001894 | ENSG00000213923 | CSNK1E |
| NM_004384 | ENSG00000151292 | CSNK1G3 |
| NM_006574 | ENSG00000114646 | CSPG5 |
| NM_030809 | ENSG00000110925 | CSRNP2 |
| NM_000100 | | CSTB |
| NM_001326 | ENSG00000176102 | CSTF3 |
| NM_001329 | | CTBP2 |
| NM_003798 | ENSG00000119326 | CTNNAL1 |
| NM_001904 | ENSG00000168036 | CTNNB1 |
| NM_001331 | ENSG00000198561 | CTNND1 |
| NM_005231 | ENSG00000085733 | CTTN |
| NM_206833 | ENSG00000178531 | CTXN1 |
| NM_003588 | ENSG00000158290 | CUL4B |
| NM_015089 | ENSG00000112659 | CUL9 |
| NM_001913 | ENSG00000257923 | CUX1 |
| NM_018294 | ENSG00000095485 | CWF19L1 |
| NM_019885 | ENSG00000003137 | CYP26B1 |
| NM_000786 | ENSG00000001630 | CYP51A1 |
| NM_001554 | ENSG00000142871 | CYR61 |
| NM_004762 | ENSG00000108669 | CYTH1 |
| NM_004393 | ENSG00000173402 | DAG1 |
| NM_139179 | ENSG00000164535 | DAGLB |
| NM_018114 | ENSG00000178149 | DALRD3 |
| NR_130730 | ENSG00000235244 | DANT2 |
| NM_018959 | ENSG00000071626 | DAZAP1 |
| NR_027642 | | DCAF13P3 |
| NM_024819 | ENSG00000172992 | DCAKD |
| NM_152624 | ENSG00000172795 | DCP2 |
| NM_004082 | ENSG00000204843 | DCTN1 |
| NM_004398 | ENSG00000178105 | DDX10 |
| NM_006386 | ENSG00000100201 | DDX17 |
| NM_018332 | ENSG00000168872 | DDX19A |
| NM_004728 | ENSG00000165732 | DDX21 |
| NM_001356 | ENSG00000215301 | DDX3X |
| NM_014829 | ENSG00000145833 | DDX46 |
| NM_004396 | ENSG00000108654 | DDX5 |
| NM_020936 | ENSG00000111364 | DDX55 |
| NM_020664 | ENSG00000242612 | DECR2 |
| NM_003472 | ENSG00000124795 | DEK |
| NM_015213 | ENSG00000184014 | DENND5A |
| NR_046909 | ENSG00000255867 | DENND5B-AS1 |
| NM_024295 | ENSG00000136986 | DERL1 |
| NM_198512 | ENSG00000184210 | DGAT2L6 |
| NM_003648 | ENSG00000077044 | DGKD |
| NM_014762 | ENSG00000116133 | DHCR24 |
| NM_014681 | ENSG00000134815 | DHX34 |
| NM_020865 | ENSG00000174953 | DHX36 |
| NM_005219 | ENSG00000131504 | DIAPH1 |
| NR_046539 | ENSG00000227528 | DIAPH3-AS1 |
| NM_014388 | ENSG00000117597 | DIEXF |
| NM_015151 | ENSG00000160305 | DIP2A |
| NM_001931 | ENSG00000150768 | DLAT |
| NM_005887 | ENSG00000176124 | DLEU1 |
| NM_004087 | ENSG00000075711 | DLG1 |
| NM_001364 | ENSG00000150672 | DLG2 |
| NR_046586 | ENSG00000231651 | DLG3-AS1 |
| NR_024585 | | DLG5-AS1 |
| NM_001933 | ENSG00000119689 | DLST |
| NM_001373 | ENSG00000185842 | DNAH14 |
| NM_001539 | | DNAJA1 |
| NM_005494 | ENSG00000105993 | DNAJB6 |
| NM_003315 | ENSG00000168259 | DNAJC7 |
| NM_005223 | | DNASE1 |
| NM_032482 | ENSG00000104885 | DOT1L |
| NM_080750 | | DPH3P1 |
| NM_013379 | ENSG00000176978 | DPP7 |
| NM_145038 | ENSG00000157856 | DRC1 |
| NM_013235 | ENSG00000113360 | DROSHA |
| NM_024918 | ENSG00000149636 | DSN1 |
| NM_021907 | ENSG00000138101 | DTNB |
| NM_022156 | | DUS1L |
| NM_030640 | ENSG00000111266 | DUSP16 |
| NM_001394 | ENSG00000120875 | DUSP4 |
| NM_001376 | ENSG00000197102 | DYNC1H1 |
| NM_005225 | ENSG00000101412 | E2F1 |
| NM_001949 | ENSG00000112242 | E2F3 |
| NM_203394 | ENSG00000165891 | E2F7 |
| NM_018029 | ENSG00000255423 | EBLN2 |
| NM_003797 | ENSG00000074266 | EED |
| NM_001960 | ENSG00000104529 | EEF1D |
| NM_018100 | ENSG00000096093 | EFHC1 |
| NM_001962 | ENSG00000184349 | EFNA5 |
| NM_004429 | ENSG00000090776 | EFNB1 |
| NM_017555 | ENSG00000269858 | EGLN2 |
| NM_014601 | ENSG00000024422 | EHD2 |
| NM_001039765 | ENSG00000281796 | EHMT1-IT1 |
| NM_014335 | ENSG00000255302 | EID1 |
| NM_001008394 | ENSG00000255150 | EID3 |
| NM_003758 | ENSG00000104131 | EIF3J |
| NM_001417 | ENSG00000063046 | EIF4B |
| NM_004095 | ENSG00000187840 | EIF4EBP1 |
| NM_003760 | ENSG00000075151 | EIF4G3 |
| NM_024930 | ENSG00000164181 | ELOVL7 |
| NM_006067 | ENSG00000131148 | EMC8 |
| NM_000117 | ENSG00000102119 | EMD |
| NM_152463 | ENSG00000154920 | EME1 |
| NM_001423 | | EMP1 |
| NM_001424 | ENSG00000213853 | EMP2 |
| NM_020193 | ENSG00000158636 | EMSY |
| NM_001242699 | ENSG00000188316 | ENO4 |
| NM_017512 | ENSG00000132199 | ENOSF1 |
| NM_004436 | ENSG00000143420 | ENSA |
| NM_004437 | ENSG00000159023 | EPB41 |
| NM_013333 | ENSG00000063245 | EPN1 |
| NM_178039 | ENSG00000082805 | ERC1 |
| NM_000122 | ENSG00000163161 | ERCC3 |
| NM_015966 | ENSG00000125991 | ERGIC3 |
| NM_207332 | ENSG00000104714 | ERICH1 |
| NM_006459 | | ERLIN1 |
| NM_024896 | ENSG00000099219 | ERMP1 |
| NM_015292 | ENSG00000139641 | ESYT1 |
| NM_031279 | ENSG00000164089 | ETNPPL |
| NM_018166 | ENSG00000142694 | EVA1B |
| NM_015189 | ENSG00000144036 | EXOC6B |
| NM_014285 | ENSG00000130713 | EXOSC2 |
| NM_058219 | | EXOSC6 |
| NM_004456 | ENSG00000106462 | EZH2 |
| NR_102425 | | EZR-AS1 |
| NM_182705 | ENSG00000183688 | FAM101B |
| NM_019018 | | FAM105A |
| NM_144635 | ENSG00000175182 | FAM131A |
| NM_152789 | ENSG00000234545 | FAM133B |
| NM_014883 | ENSG00000138640 | FAM13A |
| NM_015159 | ENSG00000054965 | FAM168A |
| NM_001009993 | ENSG00000152102 | FAM168B |
| NM_001105282 | ENSG00000164556 | FAM183BP |
| NM_032130 | ENSG00000135436 | FAM186B |
| NM_003704 | ENSG00000125386 | FAM193A |
| NM_207368 | ENSG00000225663 | FAM195B |
| NM_001039762 | ENSG00000188916 | FAM196A |
| NM_207318 | ENSG00000123575 | FAM199X |
| NM_015224 | ENSG00000163946 | FAM208A |
| NM_017782 | ENSG00000108021 | FAM208B |
| NM_021806 | ENSG00000071889 | FAM3A |
| NM_001013622 | ENSG00000174137 | FAM53A |
| NR_120630 | | FAM53B-AS1 |
| NM_016255 | ENSG00000137414 | FAM8A1 |
| NM_000135 | ENSG00000187741 | FANCA |
| NM_152633 | ENSG00000181544 | FANCB |
| NM_018062 | ENSG00000115392 | FANCL |
| NM_014808 | ENSG00000006607 | FARP2 |
| NM_004104 | ENSG00000169710 | FASN |
| NM_005245 | ENSG00000083857 | FAT1 |
| NM_022452 | ENSG00000156860 | FBRS |
| NM_012158 | ENSG00000005812 | FBXL3 |
| NM_032807 | ENSG00000134452 | FBXO18 |
| NR_003136 | | FBXO22-AS1 |
| NM_012176 | ENSG00000151876 | FBXO4 |
| NM_012347 | ENSG00000112146 | FBXO9 |
| NM_022039 | ENSG00000107829 | FBXW4 |
| NM_138782 | ENSG00000157107 | FCHO2 |
| NM_004111 | ENSG00000168496 | EEN1 |
| NM_002005 | ENSG00000182511 | FES |
| NM_004113 | ENSG00000114279 | FGF12 |
| NM_004114 | ENSG00000129682 | FGF13 |
| NM_000142 | ENSG00000068078 | FGFR3 |
| NM_001449 | ENSG00000022267 | FHL1 |
| NM_007076 | ENSG00000198855 | FICD |
| NR_026975 | ENSG00000213468 | FIRRE |
| NM_021939 | ENSG00000141756 | FKBP10 |
| NM_004470 | ENSG00000173486 | FKBP2 |
| NM_002014 | ENSG00000004478 | FKBP4 |
| NM_024301 | ENSG00000181027 | FKRP |
| NM_001456 | ENSG00000196924 | FLNA |
| NM_001457 | ENSG00000136068 | FLNB |
| NM_052905 | ENSG00000157827 | FMNL2 |
| NM_002024 | ENSG00000102081 | FMR1 |
| NM_014923 | ENSG00000102531 | FNDC3A |
| NM_004514 | ENSG00000141568 | FOXK2 |
| NM_005197 | ENSG00000053254 | FOXN3 |
| NM_002015 | ENSG00000150907 | FOXO1 |
| NM_020875 | ENSG00000138759 | FRAS1 |
| NM_174938 | ENSG00000172159 | FRMD3 |
| NM_032135 | ENSG00000189139 | FSCB |
| NM_002032 | ENSG00000167996 | FTH1 |
| NM_003902 | ENSG00000162613 | FUBP1 |
| NM_032664 | ENSG00000172728 | FUT10 |
| NM_005087 | ENSG00000114416 | FXR1 |
| NM_002040 | ENSG00000154727 | GABPA |
| NM_015973 | ENSG00000069482 | GAL |
| NM_022087 | ENSG00000178234 | GALNT11 |
| NM_052917 | ENSG00000144278 | GALNT13 |
| NM_002046 | ENSG00000111640 | GAPDH |
| NM_006478 | ENSG00000185340 | GAS2L1 |
| NM_032638 | ENSG00000179348 | GATA2 |
| NM_017660 | ENSG00000167491 | GATAD2A |
| NM_004564 | ENSG00000059691 | GATB |
| NM_176818 | ENSG00000257218 | GATC |
| NM_020944 | ENSG00000070610 | GBA2 |
| NM_001485 | ENSG00000168505 | GBX2 |
| NM_005811 | ENSG00000135414 | GDF11 |
| NM_000514 | ENSG00000168621 | GDNF |
| NM_015044 | ENSG00000103365 | GGA2 |
| NR_130107 | ENSG00000281189 | GHET1 |
| NM_021081 | ENSG00000118702 | GHRH |
| NR_004431 | ENSG00000240288 | GHRLOS |
| NM_006541 | ENSG00000108010 | GLRX3 |
| NM_006877 | ENSG00000137198 | GMPR |
| NM_007353 | ENSG00000146535 | GNA12 |
| NM_004297 | ENSG00000156049 | GNA14 |
| NM_002072 | ENSG00000156052 | GNAQ |
| NM_000516 | ENSG00000087460 | GNAS |
| NM_006098 | ENSG00000204628 | GNB2L1 |
| NM_019067 | | GNL3L |
| NM_017600 | ENSG00000238105 | GOLGA2P5 |
| NM_005895 | ENSG00000090615 | GOLGA3 |
| NM_014498 | ENSG00000173905 | GOLIM4 |
| NM_022130 | ENSG00000113384 | GOLPH3 |
| NM_015530 | ENSG00000115806 | GORASP2 |
| NM_004871 | ENSG00000108587 | GOSR1 |
| NM_002079 | ENSG00000120053 | GOT1 |
| NM_004488 | | GP5 |
| NM_016363 | ENSG00000088053 | GP6 |
| NM_174931 | ENSG00000152133 | GPATCH11 |
| NM_018040 | ENSG00000092978 | GPATCH2 |
| NM_017926 | ENSG00000089916 | GPATCH2L |
| NM_001002909 | | GPATCH8 |
| NM_170699 | ENSG00000179921 | GPBAR1 |
| NM_022913 | ENSG00000062194 | GPBP1 |
| NM_004466 | ENSG00000179399 | GPC5 |
| NM_001505 | ENSG00000164850 | GPER1 |
| NM_014373 | ENSG00000173890 | GPR160 |
| NM_000581 | ENSG00000233276 | GPX1 |
| NM_001012642 | ENSG00000175318 | GRAMD2 |
| NM_181711 | ENSG00000161835 | GRASP |
| NM_012203 | ENSG00000137106 | GRHPR |
| NM_017551 | ENSG00000182771 | GRID1 |
| NR_033368 | ENSG00000156273 | GRIK1-A52 |
| NM_014619 | ENSG00000149403 | GRIK4 |
| NM_002087 | ENSG00000030582 | GRN |
| NM_014615 | ENSG00000131149 | GSE1 |
| NM_144675 | ENSG00000169181 | GSG1L |
| NM_002093 | ENSG00000082701 | GSK3B |
| NM_001512 | ENSG00000170899 | GSTA4 |
| NM_001514 | ENSG00000137947 | GTF2B |
| NM_002095 | ENSG00000197265 | GTF2E2 |
| NM_002097 | ENSG00000122034 | GTF3A |
| NM_012341 | | GTPBP4 |
| NM_176791 | ENSG00000124196 | GTSF1L |
| NM_033553 | ENSG00000197273 | GUCA2A |
| NM_207331 | ENSG00000183666 | GUSBP1 |
| NM_002105 | ENSG00000188486 | H2AFX |
| NM_004893 | ENSG00000113648 | H2AFY |
| NR_002315 | | H3F3AP4 |
| NM_001010915 | | HACD4 |
| NM_021175 | | HAMP |
| NM_005333 | ENSG00000004961 | HCCS |
| NR_046608 | | HCFC1-AS1 |
| NM_001194 | ENSG00000099822 | HCN2 |
| NM_015401 | ENSG00000061273 | HDAC7 |
| NM_018486 | ENSG00000147099 | HDAC8 |
| NM_005336 | ENSG00000115677 | HDLBP |
| NM_018063 | ENSG00000119969 | HELLS |
| NM_004667 | ENSG00000128731 | HERC2 |
| NM_138820 | ENSG00000146066 | HIGD2A |
| NM_003325 | ENSG00000100084 | HIRA |
| NM_005319 | ENSG00000187837 | HIST1H1C |
| NM_005321 | ENSG00000168298 | HIST1H1E |
| NM_021063 | ENSG00000158373 | HIST1H2BD |
| NM_080593 | | HIST1H2BK |
| NM_003530 | | HIST1H3D |
| NM_003545 | ENSG00000276966 | HIST1H4E |
| NM_003543 | ENSG00000158406 | HIST1H4H |
| NM_002114 | ENSG00000095951 | HIVEP1 |
| NM_024567 | ENSG00000147421 | HMBOX1 |
| NM_144655 | ENSG00000148357 | HMCN2 |
| NM_002129 | | HMGB2 |
| NR_002944 | | HNRNPA1P10 |
| NM_002137 | ENSG00000122566 | HNRNPA2B1 |
| NM_004499 | ENSG00000197451 | HNRNPAB |
| NM_002138 | | HNRNPD |
| NM_005463 | ENSG00000152795 | HNRNPDL |
| NM_005520 | ENSG00000169045 | HNRNPH1 |
| NM_004501 | ENSG00000153187 | HNRNPU |
| NM_007040 | ENSG00000105323 | HNRNPUL1 |
| NR_037946 | ENSG00000234857 | HNRNPUL2-BSCL2 |
| NR_033201 | ENSG00000233101 | HOXB-AS3 |
| NM_016287 | ENSG00000127483 | HP1BP3 |
| NM_012262 | ENSG00000153936 | HS2ST1 |
| NM_005114 | | HS3ST1 |
| NM_147175 | ENSG00000171004 | HS6ST2 |
| NM_005348 | ENSG00000080824 | HSP90AA1 |
| NM_006597 | ENSG00000109971 | HSPA8 |
| NM_006644 | ENSG00000120694 | HSPH1 |
| NM_031407 | ENSG00000086758 | HUWE1 |
| NM_006389 | ENSG00000149428 | HYOU1 |
| NM_016400 | ENSG00000140264 | HYPK |
| NM_015325 | ENSG00000164151 | ICE1 |
| NM_012405 | ENSG00000116237 | ICMT |
| NM_002166 | ENSG00000115738 | ID2 |
| NM_004907 | ENSG00000160888 | IER2 |
| NM_001170820 | ENSG00000244242 | IFITM10 |
| NM_000629 | ENSG00000142166 | IFNAR1 |
| NM_001550 | ENSG00000006652 | IFRD1 |
| NM_016004 | ENSG00000101052 | IFT52 |
| NM_006546 | ENSG00000159217 | IGF2BP1 |
| NM_006547 | ENSG00000136231 | IGF2BP3 |
| NM_018725 | ENSG00000056736 | IL17RB |
| NM_144717 | ENSG00000174564 | IL20RB |
| NM_152899 | ENSG00000104951 | IL4I1 |
| NM_033416 | ENSG00000136718 | IMP4 |
| NM_032727 | ENSG00000148798 | INA |
| NM_020238 | ENSG00000149503 | INCENP |
| NM_016162 | ENSG00000111653 | ING4 |
| NM_017759 | ENSG00000114933 | INO80D |
| NM_019892 | ENSG00000148384 | INPP5E |
| NM_005542 | ENSG00000186480 | INSIG1 |
| NM_020748 | ENSG00000108506 | INTS2 |
| NM_016291 | ENSG00000068745 | IP6K2 |
| NM_002271 | ENSG00000065150 | IPO5 |
| NR_121669 | | IQCJ-SCHIP1-AS1 |
| NM_014869 | ENSG00000144711 | IQSEC1 |
| NM_001569 | ENSG00000184216 | IRAK1 |
| NM_182972 | ENSG00000168264 | IRF2BP2 |
| NM_032643 | ENSG00000128604 | IRF5 |
| NM_001572 | ENSG00000185507 | IRF7 |
| NM_003749 | ENSG00000185950 | IRS2 |
| NM_003604 | ENSG00000133124 | IRS4 |
| NM_024710 | ENSG00000063241 | ISOC2 |
| NM_000419 | ENSG00000005961 | ITGA2B |
| NM_012278 | ENSG00000147166 | ITGB1BP2 |
| NM_002223 | ENSG00000123104 | ITPR2 |
| NM_003024 | ENSG00000205726 | ITSN1 |
| NM_006469 | ENSG00000116679 | IVNS1ABP |
| NM_004973 | ENSG00000008083 | JARID2 |
| NR_034097 | | JAZF1-AS1 |
| NM_004241 | ENSG00000171988 | JMJD1C |
| NM_006694 | ENSG00000143543 | JTB |
| NM_005354 | ENSG00000130522 | JUND |
| NM_030929 | | KAZALD1 |
| NR_126346 | ENSG00000253696 | KBTBD11-OT1 |
| NM_016506 | ENSG00000123444 | KBTBD4 |
| NM_003636 | ENSG00000069424 | KCNAB2 |
| NM_012284 | ENSG00000135519 | KCNH3 |
| NM_002247 | ENSG00000156113 | KCNMA1 |
| NM_024076 | ENSG00000153885 | KCTD15 |
| NM_016121 | ENSG00000136636 | KCTD3 |
| NM_198404 | | KCTD4 |
| NM_006801 | ENSG00000105438 | KDELR1 |
| NM_006855 | ENSG00000100196 | KDELR3 |
| NM_014663 | ENSG00000066135 | KDM4A |
| NM_002035 | ENSG00000119537 | KDSR |
| NM_006559 | ENSG00000121774 | KHDRBS1 |
| NM_014686 | ENSG00000166398 | KIAA0355 |
| NM_001080398 | ENSG00000136813 | K1AA0368 |
| NM_020910 | ENSG00000122778 | KIAA1549 |
| NM_030650 | ENSG00000144320 | KIAA1715 |
| NM_032435 | ENSG00000143674 | KIAA1804 |
| NM_153369 | | KIAA1919 |
| NM_133465 | ENSG00000165185 | KIAA1958 |
| NM_015074 | ENSG00000054523 | KIF1B |
| NM_194313 | ENSG00000186638 | KIF24 |
| NM_018012 | ENSG00000162849 | KIF26B |
| NM_006845 | ENSG00000142945 | KIF2C |
| NM_012310 | ENSG00000090889 | KIF4A |
| NM_004521 | ENSG00000170759 | KIF5B |
| NM_005552 | ENSG00000126214 | KLC1 |
| NM_007249 | ENSG00000118922 | KLF12 |
| NM_016270 | ENSG00000127528 | KLF2 |
| NM_014997 | ENSG00000128607 | KLHDC10 |
| NM_014315 | ENSG00000165516 | KLHDC2 |
| NM_017566 | ENSG00000104731 | KLHDC4 |
| NM_018143 | | KLHL11 |
| NM_014851 | ENSG00000162413 | KLHL21 |
| NM_032775 | ENSG00000099910 | KLHL22 |
| NM_025067 | ENSG00000119771 | KLHL29 |
| NM_017415 | ENSG00000146021 | KLHL3 |
| NM_005933 | ENSG00000118058 | KMT2A |
| NM_014727 | ENSG00000272333 | KMT2B |
| NM_021230 | ENSG00000055609 | KMT2C |
| NM_003482 | ENSG00000167548 | KMT2D |
| NM_002265 | | KPNB1 |
| NM_015478 | ENSG00000185513 | L3MBTL1 |
| NM_002286 | ENSG00000089692 | LAG3 |
| NM_018407 | ENSG00000104341 | LAPTM4B |
| NM_004737 | ENSG00000133424 | LARGE |
| NM_015155 | ENSG00000107929 | LARP4B |
| NR_048543 | | LARS2-AS1 |
| NM_004690 | ENSG00000131023 | LATS1 |
| NM_002296 | ENSG00000143815 | LBR |
| NM_182551 | ENSG00000172954 | LCLAT1 |
| NM_003893 | ENSG00000198728 | LDB1 |
| NM_002300 | ENSG00000111716 | LDHB |
| NM_002301 | ENSG00000166796 | LDHC |
| NM_004338 | ENSG00000168675 | LDLRAD4 |
| NM_181336 | ENSG00000161904 | LEMD2 |
| NM_198988 | ENSG00000275183 | LENG9 |
| NM_005567 | ENSG00000108679 | LGALS3BP |
| NM_014564 | ENSG00000107187 | LHX3 |
| NR_037642 | ENSG00000230124 | LHX4-AS1 |
| NM_002311 | ENSG00000005156 | LIG3 |
| NR_033947 | | LIMD1-AS1 |
| NM_022165 | ENSG00000104863 | LIN7B |
| NR_033376 | ENSG00000203801 | LINC00222 |
| NR_103753 | | LINC00491 |
| NR_033876 | ENSG00000227036 | LINC00511 |
| NR_027103 | ENSG00000224514 | LINC00620 |
| NR_038970 | ENSG00000258441 | LINC00641 |
| NR_028138 | ENSG00000271614 | LINC00936 |
| NR_038292 | ENSG00000281706 | LINC01012 |
| NR_024423 | ENSG00000250056 | LINC01018 |
| NR_132375 | | LINC01078 |
| NM_178529 | ENSG00000279873 | LINC01126 |
| NR_103791 | | LINC01127 |
| NR_015360 | ENSG00000245937 | LINC01184 |
| NR_110616 | | LINC01355 |
| NR_109928 | | LINC01424 |
| NR_033917 | ENSG00000230176 | LINC01433 |
| NR_110218 | ENSG00000237877 | LINC01473 |
| NM_175616 | ENSG00000236882 | LINC01554 |
| NR_039999 | ENSG00000262468 | LINC01569 |
| NR_120371 | ENSG00000245479 | LINC01585 |
| NR_125410 | ENSG00000272138 | LINC01607 |
| NM_001256373 | ENSG00000257242 | LINC01619 |
| NM_032808 | ENSG00000169783 | LINGO1 |
| NM_004140 | ENSG00000131899 | LLGL1 |
| NR_110945 | ENSG00000260439 | LMF1-AS1 |
| NM_005572 | ENSG00000160789 | LMNA |
| NM_005573 | ENSG00000113368 | LMNB1 |
| NM_005358 | ENSG00000136153 | LMO7 |
| NR_027406 | | LOC100129034 |
| NR_045112 | | LOC100129617 |
| NM_001242698 | | LOC100130357 |
| NM_001272086 | | LOC100130370 |
| NR_046285 | | LOC100130744 |
| NM_001243523 | | LOC100130880 |
| NR_024594 | ENSG00000267882 | LOC100131496 |
| NR_027069 | ENSG00000231609 | LOC100132215 |
| NM_001242885 | | LOC100287036 |
| NR_033175 | | LOC100289673 |
| NR_038333 | ENSG00000246422 | LOC100505658 |
| NR_038982 | | LOC100507346 |
| NR_038244 | ENSG00000235652 | LOC100507557 |
| NM_001278082 | ENSG00000275765 | LOC100652758 |
| NR_110102 | ENSG00000242687 | LOC101927550 |
| NR_110808 | ENSG00000266100 | LOC101927557 |
| NR_110931 | | LOC101927817 |
| NR_125892 | | LOC101928279 |
| NR_125858 | | LOC101928461 |
| NR_110092 | ENSG00000258274 | LOC101928731 |
| NR_105012 | | LOC101929154 |
| NR_123739 | ENSG00000230550 | LOC101929441 |
| NR_120366 | | LOC101929679 |
| NR_120665 | ENSG00000227495 | LOC102724009 |
| NR_120674 | ENSG00000231964 | LOC102724323 |
| NR_120684 | ENSG00000260917 | LOC103344931 |
| NR_131227 | | LOC105616981 |
| NR_033921 | ENSG00000265533 | LOC643542 |
| NR_034179 | ENSG00000231305 | LOC653712 |
| NR_003671 | | LOC728024 |
| NM_004793 | ENSG00000196365 | LONP1 |
| NM_031490 | | LONP2 |
| NM_006726 | ENSG00000198589 | LRBA |
| NM_153377 | ENSG00000139263 | LRIG3 |
| NM_002335 | ENSG00000162337 | LRP5 |
| NM_002336 | ENSG00000070018 | LRP6 |
| NM_052888 | ENSG00000185158 | LRRC37B |
| NM_018103 | ENSG00000171492 | LRRC8D |
| NM_006309 | ENSG00000093167 | LRRFIP2 |
| NM_024652 | ENSG00000154237 | LRRK1 |
| NM_152344 | ENSG00000161654 | LSM12 |
| NM_012321 | ENSG00000130520 | LSM4 |
| NM_019839 | ENSG00000213906 | LTB4R2 |
| NM_000428 | ENSG00000119681 | LTBP2 |
| NM_021070 | ENSG00000168056 | LTBP3 |
| NM_032860 | ENSG00000135521 | LTV1 |
| NM_016019 | ENSG00000146963 | LUC7L2 |
| NM_005583 | ENSG00000104903 | LYL1 |
| NM_020466 | ENSG00000083099 | LYRM2 |
| NM_003550 | ENSG00000002822 | MAD1L1 |
| NR_002819 | ENSG00000251562 | MALAT1 |
| NM_014757 | ENSG00000161021 | MAML1 |
| NM_006699 | ENSG00000198162 | MAN1A2 |
| NM_022818 | ENSG00000140941 | MAP1LC3B |
| NM_030662 | ENSG00000126934 | MAP2K2 |
| NM_004721 | ENSG00000073803 | MAP3K13 |
| NM_003188 | ENSG00000135341 | MAP3K7 |
| NM_024871 | ENSG00000180834 | MAP6D1 |
| NM_004759 | ENSG00000162889 | MAPKAPK2 |
| NM_012325 | ENSG00000101367 | MAPRE1 |
| NM_023009 | ENSG00000175130 | MARCKSL1 |
| NM_002380 | ENSG00000132561 | MATN2 |
| NM_021038 | ENSG00000152601 | MBNL1 |
| NM_018388 | ENSG00000076770 | MBNL3 |
| NM_022132 | ENSG00000131844 | MCCC2 |
| NM_006739 | ENSG00000100297 | MCM5 |
| NM_005915 | ENSG00000076003 | MCM6 |
| NM_005916 | ENSG00000166508 | MCM7 |
| NM_005918 | ENSG00000146701 | MDH2 |
| NM_002393 | ENSG00000198625 | MDM4 |
| NM_004991 | ENSG00000085276 | MECOM |
| NM_004992 | ENSG00000169057 | MECP2 |
| NM_032286 | ENSG00000133398 | MED10 |
| NM_005121 | ENSG00000108510 | MED13 |
| NM_005481 | ENSG00000175221 | MED16 |
| NM_004269 | ENSG00000160563 | MED27 |
| NM_015955 | ENSG00000162959 | MEMO1 |
| NM_000244 | ENSG00000133895 | MEN1 |
| NM_006838 | ENSG00000111142 | METAP2 |
| NM_001010977 | ENSG00000139780 | METTL21C |
| NM_024109 | ENSG00000067365 | METTL22 |
| NM_019852 | ENSG00000165819 | METTL3 |
| NM_016626 | ENSG00000176624 | MEX3C |
| NM_203304 | ENSG00000181588 | MEX3D |
| NM_004225 | ENSG00000147324 | MFHAS1 |
| NM_001120 | ENSG00000109736 | MFSD10 |
| NM_033055 | ENSG00000156875 | MFSD14A |
| NM_002413 | ENSG00000085871 | MGST2 |
| NM_033386 | ENSG00000100139 | MICALL1 |
| NM_139162 | ENSG00000177427 | MIEF2 |
| NM_002415 | | MIF |
| NM_021933 | ENSG00000116691 | MIIP |
| NR_031611 | | MIR1206 |
| NR_031595 | ENSG00000221585 | MIR1226 |
| NR_031596 | ENSG00000221411 | MIR1227 |
| NR_036262 | | MIR1244-2 |
| NR_031658 | ENSG00000221417 | MIR1257 |
| NR_031692 | | MIR1279 |
| NR_029682 | ENSG00000207708 | MIR141 |
| NR_029525 | ENSG00000198987 | MIR16-2 |
| NR_038975 | ENSG00000224020 | MIR181A2HG |
| NR_031750 | ENSG00000253030 | MIR2116 |
| NR_036056 | ENSG00000276326 | MIR2909 |
| NR_036068 | ENSG00000264358 | MIR3122 |
| NR_036075 | ENSG00000265396 | MIR3128 |
| NR_036091 | ENSG00000265623 | MIR3139 |
| NR_036117 | ENSG00000265014 | MIR3160-1 |
| NR_036152 | ENSG00000266189 | MIR3186 |
| NR_130463 | ENSG00000265306 | MIR3195 |
| NR_039851 | ENSG00000265371 | MIR3198-2 |
| NR_029506 | ENSG00000207698 | MIR32 |
| NR_029896 | | MIR324 |
| NR_029507 | ENSG00000207932 | MIR33A |
| NR_037415 | ENSG00000264944 | MIR3620 |
| NR_037424 | ENSG00000281156 | MIR3651 |
| NR_037425 | ENSG00000265072 | MIR3652 |
| NR_037427 | | MIR3654 |
| NR_037430 | ENSG00000266370 | MIR3657 |
| NR_037431 | | MIR3658 |
| NR_037450 | ENSG00000263813 | MIR3679 |
| NR_037465 | ENSG00000264818 | MIR3714 |
| NR_039667 | ENSG00000263361 | MIR378H |
| NR_037486 | ENSG00000264897 | MIR3921 |
| NR_037498 | ENSG00000266509 | MIR3934 |
| NR_030398 | | MIR421 |
| NR_036177 | ENSG00000264763 | MIR4295 |
| NR_036197 | ENSG00000265195 | MIR4312 |
| NR_039624 | | MIR4426 |
| NR_039626 | ENSG00000266262 | MIR4428 |
| NR_039646 | ENSG00000263721 | MIR4444-1 |
| NR_039662 | ENSG00000263670 | MIR4457 |
| NR_039664 | ENSG00000265421 | MIR4459 |
| NR_039666 | ENSG00000263963 | MIR4461 |
| NR_039676 | ENSG00000271899 | MIR4466 |
| NR_039685 | ENSG00000264941 | MIR4474 |
| NR_039719 | ENSG00000266704 | MIR4498 |
| NR_030255 | ENSG00000207726 | MIR455 |
| NR_039787 | ENSG00000266245 | MIR4644 |
| NR_039790 | ENSG00000265700 | MIR4647 |
| NR_039814 | ENSG00000266315 | MIR4668 |
| NR_039819 | ENSG00000263979 | MIR4672 |
| NR_039849 | ENSG00000265455 | MIR4700 |
| NR_039902 | ENSG00000263409 | MIR4747 |
| NR_039903 | ENSG00000265879 | MIR4748 |
| NR_039915 | ENSG00000265329 | MIR4758 |
| NR_039964 | ENSG00000265080 | MIR4800 |
| NR_039967 | ENSG00000264099 | MIR4803 |
| NR_039968 | ENSG00000263593 | MIR4804 |
| NR_030166 | | MIR491 |
| NR_039912 | | MIR499B |
| NR_039969 | ENSG00000266241 | MIR5047 |
| NR_049816 | ENSG00000266307 | MIR5093 |
| NR_039973 | ENSG00000266270 | MIR5096 |
| NR_036088 | ENSG00000265981 | MIR544B |
| NR_030258 | ENSG00000207820 | MIR545 |
| NR_039621 | ENSG00000264419 | MIR548AC |
| NR_039629 | ENSG00000265301 | MIR548AD |
| NR_049853 | | MIR548AU |
| NR_031677 | ENSG00000221537 | MIR548H1 |
| NR_036071 | ENSG00000265056 | MIR548S |
| NR_036103 | ENSG00000265520 | MIR548V |
| NR_049846 | ENSG00000263540 | MIR5582 |
| NR_049851 | ENSG00000263629 | MIR5586 |
| NR_049866 | ENSG00000264056 | MIR5685 |
| NR_049880 | ENSG00000266721 | MIR5695 |
| NR_106713 | ENSG00000276162 | MIR5739 |
| NR_030305 | ENSG00000207956 | MIR579 |
| NR_030313 | ENSG00000207769 | MIR586 |
| NR_030318 | ENSG00000207973 | MIR589 |
| NR_030321 | ENSG00000207741 | MIR590 |
| NR_030324 | ENSG00000207588 | MIR593 |
| NR_030333 | ENSG00000207693 | MIR602 |
| NR_106718 | ENSG00000278433 | MIR6070 |
| NR_030343 | ENSG00000273834 | MIR612 |
| NR_106745 | ENSG00000273500 | MIR6129 |
| NR_106748 | ENSG00000275870 | MIR6132 |
| NR_030351 | ENSG00000207967 | MIR620 |
| NR_030356 | ENSG00000207766 | MIR626 |
| NR_030366 | ENSG00000207556 | MIR636 |
| NR_030374 | ENSG00000207997 | MIR644A |
| NR_106997 | ENSG00000281678 | MIR6516 |
| NR_106773 | ENSG00000275466 | MIR6716 |
| NR_106778 | ENSG00000275859 | MIR6720 |
| NR_106786 | ENSG00000274258 | MIR6728 |
| NR_106805 | ENSG00000276102 | MIR6747 |
| NR_106824 | ENSG00000275101 | MIR6766 |
| NR_106840 | ENSG00000275107 | MIR6782 |
| NR_106841 | ENSG00000278223 | MIR6783 |
| NR_106845 | ENSG00000275505 | MIR6787 |
| NR_106850 | ENSG00000273657 | MIR6792 |
| NR_106854 | ENSG00000275652 | MIR6796 |
| NR_106865 | ENSG00000275924 | MIR6807 |
| NR_106877 | ENSG00000278420 | MIR6819 |
| NR_106909 | ENSG00000274673 | MIR6850 |
| NR_106914 | ENSG00000276124 | MIR6855 |
| NR_106916 | ENSG00000278204 | MIR6857 |
| NR_106929 | ENSG00000276741 | MIR6869 |
| NR_106937 | ENSG00000273932 | MIR6877 |
| NR_106938 | | MIR6878 |
| NR_106940 | ENSG00000275967 | MIR6880 |
| NR_106946 | ENSG00000273892 | MIR6886 |
| NR_106948 | ENSG00000275141 | MIR6888 |
| NR_106949 | ENSG00000274552 | MIR6889 |
| NR_106960 | ENSG00000275891 | MIR7110 |
| NR_106981 | ENSG00000278571 | MIR7161 |
| NR_031757 | ENSG00000211524 | MIR718 |
| NR_106988 | | MIR7641-2 |
| NR_107030 | ENSG00000277202 | MIR8063 |
| NR_107035 | ENSG00000273912 | MIR8068 |
| NR_107042 | ENSG00000277942 | MIR8075 |
| NR_024391 | ENSG00000267374 | MIR924HG |
| NR_030760 | ENSG00000216083 | MIR936 |
| NR_030637 | | MIR941-1 |
| NR_030640 | ENSG00000215930 | MIR942 |
| NR_030641 | ENSG00000216105 | MIR943 |
| NR_029484 | ENSG00000208012 | MIRLET7F2 |
| NM_018353 | ENSG00000129534 | MIS18BP1 |
| NM_002417 | ENSG00000148773 | MKI67 |
| NM_020831 | ENSG00000196588 | MKL1 |
| NM_017572 | ENSG00000099875 | MKNK2 |
| NM_014160 | ENSG00000075975 | MKRN2 |
| NM_014730 | ENSG00000110917 | MLEC |
| NM_000249 | ENSG00000076242 | MLH1 |
| NM_014381 | ENSG00000119684 | MLH3 |
| NM_004641 | ENSG00000078403 | MLLT10 |
| NM_032951 | ENSG00000009950 | MLXIPL |
| NR_102705 | | MMP24-AS1 |
| NM_198468 | ENSG00000146263 | MMS22L |
| NM_002430 | ENSG00000169184 | MN1 |
| NM_006791 | ENSG00000185787 | MORF4L1 |
| NM_012286 | ENSG00000123562 | MORF4L2 |
| NM_020963 | ENSG00000155363 | MOV10 |
| NM_002434 | ENSG00000103152 | MPG |
| NM_005792 | ENSG00000135698 | MPHOSPH6 |
| NM_138701 | | MPLKIP |
| NM_001932 | ENSG00000161647 | MPP3 |
| NM_015134 | ENSG00000133030 | MPRIP |
| NM_033296 | ENSG00000179010 | MRFAP1 |
| NM_152301 | ENSG00000178988 | MRFAP1L1 |
| NM_018270 | | MRGBP |
| NM_014078 | ENSG00000172172 | MRPL13 |
| NM_032111 | ENSG00000180992 | MRPL14 |
| NM_024540 | ENSG00000143314 | MRPL24 |
| NR_002208 | | MRPL42P5 |
| NM_016640 | ENSG00000112996 | MRPS30 |
| NM_020662 | ENSG00000124532 | MRS2 |
| NM_001012982 | | MSANTD1 |
| NM_006745 | ENSG00000052802 | MSMO1 |
| NM_002444 | ENSG00000147065 | MSN |
| NR_024117 | | MSTO2P |
| NM_002451 | ENSG00000099810 | MTAP |
| NM_025198 | ENSG00000120832 | MTERF2 |
| NM_007358 | ENSG00000143033 | MTF2 |
| NM_138419 | ENSG00000146410 | MTFR2 |
| NM_015440 | ENSG00000120254 | MTHFD1L |
| NM_145808 | ENSG00000105887 | MTPN |
| NM_000254 | ENSG00000116984 | MTR |
| NM_138383 | ENSG00000132613 | MTSS1L |
| NM_020749 | ENSG00000129422 | MTUS1 |
| NR_046378 | | MTUS2-AS1 |
| NM_005961 | ENSG00000184956 | MUC6 |
| NM_005115 | ENSG00000013364 | MVP |
| NM_002466 | ENSG00000101057 | MYBL2 |
| NM_002467 | ENSG00000136997 | MYC |
| NR_046716 | ENSG00000236051 | MYCBP2-AS1 |
| NM_002474 | ENSG00000133392 | MYH11 |
| NM_021019 | ENSG00000092841 | MYL6 |
| NM_018657 | ENSG00000085274 | MYNN |
| NM_005379 | ENSG00000166866 | MYO1A |
| NM_004145 | ENSG00000099331 | MYO9B |
| NM_025146 | ENSG00000121579 | NAA50 |
| NM_005594 | ENSG00000196531 | NACA |
| NM_052876 | ENSG00000160877 | NACC1 |
| NM_199461 | ENSG00000188613 | NANOS1 |
| NM_004537 | ENSG00000187109 | NAP1L1 |
| NM_145201 | ENSG00000147813 | NAPRT |
| NM_024662 | ENSG00000135372 | NAT10 |
| NM_145117 | ENSG00000166833 | NAV2 |
| NM_198945 | ENSG00000144426 | NBEAL1 |
| NM_022346 | ENSG00000109805 | NCAPG |
| NM_017760 | ENSG00000146918 | NCAPG2 |
| NM_018553 | | NCBP3 |
| NM_016453 | ENSG00000213672 | NCKIPSD |
| NM_014071 | ENSG00000198646 | NCOA6 |
| NM_030808 | ENSG00000166579 | NDEL1 |
| NM_014434 | ENSG00000188566 | NDOR1 |
| NM_020465 | ENSG00000103034 | NDRG4 |
| NM_016013 | ENSG00000137806 | NDUFAF1 |
| NR_002802 | | NEAT1 |
| NM_018090 | ENSG00000157191 | NECAP2 |
| NM_133494 | ENSG00000151414 | NEK7 |
| NM_004713 | | NEMF |
| NM_018092 | ENSG00000171208 | NETO2 |
| NR_120675 | ENSG00000235470 | NEURL1-AS1 |
| NM_004555 | ENSG00000072736 | NFATC3 |
| NM_003204 | ENSG00000082641 | NFE2L1 |
| NR_104180 | ENSG00000237853 | NFIA-AS1 |
| NM_005597 | ENSG00000141905 | NFIC |
| NM_002501 | ENSG00000008441 | NFIX |
| NM_002504 | ENSG00000086102 | NFX1 |
| NM_015514 | ENSG00000129460 | NGDN |
| NM_014380 | ENSG00000166681 | NGFRAP1 |
| NM_016350 | ENSG00000100503 | NIN |
| NM_015384 | ENSG00000164190 | NIPBL |
| NM_020202 | | NIT2 |
| NM_173522 | ENSG00000233382 | NKAPP1 |
| NM_016231 | ENSG00000087095 | NLK |
| NM_002512 | ENSG00000011052 | NME2 |
| NM_022787 | ENSG00000173614 | NMNAT1 |
| NM_005386 | ENSG00000053438 | NNAT |
| NM_022451 | ENSG00000173145 | NOC3L |
| NM_016167 | | NOL7 |
| NM_004741 | ENSG00000166197 | NOLC1 |
| NM_003703 | ENSG00000087269 | NOP14 |
| NM_002517 | ENSG00000130751 | NPAS1 |
| NM_000271 | ENSG00000141458 | NPC1 |
| NM_015392 | ENSG00000107281 | NPDC1 |
| NM_017921 | ENSG00000182446 | NPLOC4 |
| NM_002520 | ENSG00000181163 | NPM1 |
| NM_002522 | ENSG00000171246 | NPTX1 |
| NM_021724 | ENSG00000126368 | NR1D1 |
| NM_005126 | ENSG00000174738 | NR1D2 |
| NM_003889 | ENSG00000144852 | NR1I2 |
| NR_024046 | | NRADDP |
| NM_002524 | ENSG00000213281 | NRAS |
| NM_002525 | ENSG00000078618 | NRDC |
| NM_005011 | ENSG00000106459 | NRF1 |
| NM_173685 | ENSG00000156831 | NSMCE2 |
| NM_014595 | ENSG00000125458 | NT5C |
| NM_020201 | ENSG00000205309 | NT5M |
| NM_173474 | ENSG00000157045 | NTAN1 |
| NM_014064 | ENSG00000148335 | NTMT1 |
| NM_030952 | ENSG00000163545 | NUAK2 |
| NR_046633 | ENSG00000235191 | NUCB1-AS1 |
| NM_022731 | ENSG00000069275 | NUCKS1 |
| NM_032869 | ENSG00000120526 | NUDCD1 |
| NM_015332 | | NUDCD3 |
| NM_020772 | ENSG00000108256 | NUFIP2 |
| NM_015231 | ENSG00000030066 | NUP160 |
| NM_024923 | ENSG00000132182 | NUP210 |
| NM_005085 | ENSG00000126883 | NUP214 |
| NM_007172 | ENSG00000093000 | NUP50 |
| NM_138459 | ENSG00000153989 | NUS1 |
| NM_006362 | ENSG00000162231 | NXF1 |
| NM_022463 | ENSG00000167693 | NXN |
| NM_004152 | ENSG00000104904 | OAZ1 |
| NM_015311 | ENSG00000124006 | OBSL1 |
| NM_152635 | ENSG00000138315 | OIT3 |
| NM_025136 | ENSG00000125741 | OPA3 |
| NM_001708 | ENSG00000128617 | OPN1SW |
| NM_000607 | ENSG00000229314 | ORM1 |
| NM_014182 | | ORMDL2 |
| NR_049771 | ENSG00000232490 | OSBPL10-AS1 |
| NM_017670 | ENSG00000167770 | OTUB1 |
| NM_002560 | ENSG00000135124 | P2RX4 |
| NM_002568 | ENSG00000070756 | PABPC1 |
| NM_030979 | | PABPC3 |
| NM_004643 | | PABPN1 |
| NM_145048 | ENSG00000163138 | PACRGL |
| NM_000430 | ENSG00000007168 | PAFAH1B1 |
| NM_016480 | ENSG00000120727 | PAIP2 |
| NM_000919 | ENSG00000145730 | PAM |
| NM_006999 | ENSG00000112941 | PAPD7 |
| NM_173462 | ENSG00000100767 | PAPLN |
| NM_019619 | ENSG00000148498 | PARD3 |
| NM_018622 | ENSG00000175193 | PARL |
| NM_001618 | ENSG00000143799 | PARP1 |
| NM_017851 | ENSG00000138617 | PARP16 |
| NM_013327 | ENSG00000188677 | PARVB |
| NM_002583 | | PAWR |
| NM_022129 | ENSG00000108187 | PBLD |
| NM_002585 | ENSG00000185630 | PBX1 |
| NM_006195 | ENSG00000167081 | PBX3 |
| NM_025245 | ENSG00000105717 | PBX4 |
| NR_109828 | | PCBP2-OT1 |
| NM_018929 | ENSG00000240764 | PCDHGC5 |
| NM_032373 | ENSG00000180628 | PCGF5 |
| NM_020357 | ENSG00000081154 | PCNP |
| NM_006031 | ENSG00000160299 | PCNT |
| NM_032346 | ENSG00000126249 | PDCD2L |
| NM_004708 | ENSG00000105185 | PDCD5 |
| NM_013374 | ENSG00000170248 | PDCD6IP |
| NM_002599 | ENSG00000186642 | PDE2A |
| NM_000921 | ENSG00000172572 | PDE3A |
| NM_002605 | ENSG00000073417 | PDE8A |
| NM_006849 | ENSG00000185615 | PDIA2 |
| NM_015200 | ENSG00000121892 | PDS5A |
| NM_003681 | ENSG00000160209 | PDXK |
| NM_173791 | | PDZD8 |
| NM_002567 | ENSG00000089220 | PEBP1 |
| NM_138575 | ENSG00000247077 | PGAM5 |
| NM_000291 | | PGK1 |
| NM_006667 | ENSG00000101856 | PGRMC1 |
| NM_024419 | ENSG00000087157 | PGS1 |
| NM_014660 | ENSG00000106443 | PHF14 |
| NM_015651 | ENSG00000119403 | PHF19 |
| NM_005392 | ENSG00000197724 | PHF2 |
| NM_016436 | ENSG00000025293 | PHF20 |
| NM_024297 | ENSG00000040633 | PHF23 |
| NM_006608 | ENSG00000116793 | PHTF1 |
| NM_174933 | ENSG00000175287 | PHYHD1 |
| NM_153370 | ENSG00000164530 | PI16 |
| NM_017933 | ENSG00000153823 | PID1 |
| NM_002645 | ENSG00000011405 | PIK3C2A |
| NR_126366 | ENSG00000231789 | PIK3CD-AS2 |
| NM_005027 | ENSG00000105647 | PIK3R2 |
| NM_014602 | ENSG00000196455 | PIK3R4 |
| NR_003571 | | PIN4P1 |
| NM_003559 | ENSG00000276293 | PIP4K2B |
| NM_012417 | ENSG00000154217 | PITPNC1 |
| NM_001199924 | ENSG00000260804 | PKI55 |
| NM_003706 | ENSG00000105499 | PLA2G4C |
| NM_021796 | ENSG00000170965 | PLAC1 |
| NM_001029869 | ENSG00000173261 | PLAC8L1 |
| NM_178836 | ENSG00000179598 | PLD6 |
| NM_000445 | ENSG00000178209 | PLEC |
| NM_019012 | ENSG00000052126 | PLEKHA5 |
| NM_015993 | ENSG00000102934 | PLLP |
| NM_022737 | ENSG00000105520 | PLPPR2 |
| NM_005032 | ENSG00000102024 | PLS3 |
| NM_032242 | ENSG00000114554 | PLXNA1 |
| NM_002673 | ENSG00000164050 | PLXNB1 |
| NM_002676 | ENSG00000100417 | PMM1 |
| NM_015160 | ENSG00000165688 | PMPCA |
| NM_002687 | ENSG00000100941 | PNN |
| NM_015720 | ENSG00000114631 | PODXL2 |
| NM_015227 | ENSG00000186866 | POFUT2 |
| NM_017542 | | POGK |
| NM_015100 | ENSG00000143442 | POGZ |
| NM_021173 | ENSG00000175482 | POLD4 |
| NM_002693 | ENSG00000140521 | POLG |
| NM_019014 | ENSG00000125630 | POLR1B |
| NM_006232 | ENSG00000163882 | POLR2H |
| NM_138338 | ENSG00000100413 | POLR3H |
| NM_017739 | ENSG00000085998 | POMGNT1 |
| NM_006237 | ENSG00000152192 | POU4F1 |
| NM_153216 | ENSG00000248483 | POU5F2 |
| NM_006903 | ENSG00000138777 | PPA2 |
| NM_133263 | ENSG00000155846 | PPARGC1B |
| NM_002706 | ENSG00000138032 | PPM1B |
| NM_020700 | ENSG00000111110 | PPM1H |
| NM_144641 | ENSG00000164088 | PPM1M |
| NM_002710 | ENSG00000186298 | PPP1CC |
| NM_002481 | ENSG00000077157 | PPP1R12B |
| NM_001007533 | ENSG00000182676 | PPP1R27 |
| NM_002716 | ENSG00000137713 | PPP2R1B |
| NM_021132 | ENSG00000107758 | PPP3CB |
| NM_005605 | ENSG00000120910 | PPP3CC |
| NM_005134 | ENSG00000154845 | PPP4R1 |
| NM_014678 | ENSG00000100239 | PPP6R2 |
| NM_018312 | ENSG00000110075 | PPP6R3 |
| NM_017765 | ENSG00000040487 | PQLC2 |
| NM_032152 | ENSG00000133246 | PRAM1 |
| NR_051984 | ENSG00000258725 | PRC1-AS1 |
| NM_013388 | ENSG00000138073 | PREB |
| NM_006553 | ENSG00000141391 | PRELID3A |
| NM_153026 | ENSG00000139174 | PRICKLE1 |
| NM_002733 | ENSG00000181929 | PRKAG1 |
| NM_002734 | ENSG00000108946 | PRKAR1A |
| NM_002735 | ENSG00000188191 | PRKAR1B |
| NR_110822 | | PRKCA-AS1 |
| NM_005400 | ENSG00000171132 | PRKCE |
| NM_003891 | ENSG00000126231 | PROZ |
| NM_018061 | ENSG00000134186 | PRPF38B |
| NM_017892 | ENSG00000196504 | PRPF40A |
| NM_012469 | ENSG00000101161 | PRPF6 |
| NM_020719 | ENSG00000126464 | PRR12 |
| NM_013318 | ENSG00000130723 | PRRC2B |
| NM_015172 | ENSG00000117523 | PRRC2C |
| NM_145239 | ENSG00000167371 | PRRT2 |
| NM_000021 | ENSG00000080815 | PSEN1 |
| NM_021144 | ENSG00000164985 | PSIP1 |
| NM_002788 | ENSG00000100567 | PSMA3 |
| NM_002789 | ENSG00000041357 | PSMA4 |
| NM_002795 | ENSG00000277791 | PSMB3 |
| NM_002796 | ENSG00000159377 | PSMB4 |
| NM_002799 | ENSG00000136930 | PSMB7 |
| NM_002805 | ENSG00000087191 | PSMC5 |
| NM_002815 | ENSG00000108671 | PSMD11 |
| NM_002816 | | PSMD12 |
| NM_002808 | ENSG00000175166 | PSMD2 |
| NM_003720 | ENSG00000183527 | PSMG1 |
| NM_001128591 | ENSG00000180822 | PSMG4 |
| NM_030664 | ENSG00000165983 | PTER |
| NM_020440 | ENSG00000134247 | PTGFRN |
| NM_005607 | ENSG00000169398 | PTK2 |
| NM_003463 | ENSG00000112245 | PTP4A1 |
| NM_003479 | ENSG00000184007 | PTP4A2 |
| NM_002834 | ENSG00000179295 | PTPN11 |
| NM_014369 | ENSG00000072135 | PTPN18 |
| NM_015466 | ENSG00000076201 | PTPN23 |
| NM_002850 | ENSG00000105426 | PTPRS |
| NM_004339 | ENSG00000183255 | PTTG1IP |
| NM_015317 | ENSG00000055917 | PUM2 |
| NM_013357 | ENSG00000172733 | PURG |
| NM_031292 | ENSG00000129317 | PUS7L |
| NM_012293 | ENSG00000130508 | PXDN |
| NM_002859 | ENSG00000089159 | PXN |
| NR_038924 | ENSG00000255857 | PXN-AS1 |
| NM_002863 | ENSG00000100504 | PYGL |
| NM_005609 | ENSG00000068976 | PYGM |
| NM_015617 | ENSG00000171016 | PYGO1 |
| NM_198180 | ENSG00000188710 | QRFP |
| NM_002826 | ENSG00000116260 | QSOX1 |
| NM_014925 | ENSG00000179912 | R3HDM2 |
| NM_025151 | ENSG00000156675 | RAB11FIP1 |
| NM_016322 | ENSG00000119396 | RAB14 |
| NM_014999 | | RAB21 |
| NM_004249 | ENSG00000157869 | RAB28 |
| NM_001031834 | | RAB40AL |
| NM_004637 | ENSG00000075785 | RAB7A |
| NM_005370 | | RAB8A |
| NM_006908 | ENSG00000136238 | RAC1 |
| NM_005053 | ENSG00000179262 | RAD23A |
| NM_002874 | ENSG00000119318 | RAD23B |
| NM_134422 | ENSG00000002016 | RAD52 |
| NM_006550 | ENSG00000197275 | RAD54B |
| NM_015106 | ENSG00000164080 | RAD54L2 |
| NR_130894 | ENSG00000237328 | RAI1-AS1 |
| NM_006266 | ENSG00000160271 | RALGDS |
| NM_002884 | ENSG00000116473 | RAP1A |
| NM_015646 | ENSG00000127314 | RAP1B |
| NM_016340 | ENSG00000158987 | RAPGEF6 |
| NM_016339 | ENSG00000108352 | RAPGEFL1 |
| NM_005055 | ENSG00000165917 | RAPSN |
| NM_020320 | ENSG00000146282 | RARS2 |
| NM_006506 | ENSG00000155903 | RASA2 |
| NM_018211 | ENSG00000162437 | RAVER2 |
| NM_006910 | ENSG00000122257 | RBBP6 |
| NM_014309 | ENSG00000100320 | RBFOX2 |
| NM_022768 | ENSG00000162775 | RBM15 |
| NM_018605 | ENSG00000139746 | RBM26 |
| NM_004902 | ENSG00000131051 | RBM39 |
| NM_002896 | ENSG00000173933 | RBM4 |
| NM_031492 | ENSG00000173914 | RBM4B |
| NM_014248 | | RBX1 |
| NM_018715 | ENSG00000179051 | RCC2 |
| NM_002902 | | RCN2 |
| NM_016606 | ENSG00000132563 | REEP2 |
| NM_001001330 | ENSG00000165476 | REEP3 |
| NM_032871 | ENSG00000054967 | RELT |
| NM_013400 | ENSG00000214022 | REPIN1 |
| NM_004726 | ENSG00000169891 | REPS2 |
| NM_020695 | ENSG00000079313 | REXO1 |
| NM_015523 | ENSG00000076043 | REXO2 |
| NM_002913 | ENSG00000035928 | RFC1 |
| NM_002915 | ENSG00000133119 | RFC3 |
| NM_002919 | ENSG00000080298 | RFX3 |
| NM_020211 | ENSG00000182175 | RGNM_ |
| NM_005614 | ENSG00000106615 | RHEB |
| NM_001252499 | ENSG00000171792 | RHNO1 |
| NM_004040 | ENSG00000143878 | RHOB |
| NM_152756 | ENSG00000164327 | RICTOR |
| NM_018151 | ENSG00000080345 | RIF1 |
| NM_012421 | ENSG00000117000 | RLF |
| NM_001013838 | ENSG00000159753 | RLTPR |
| NM_018145 | ENSG00000137824 | RMDN3 |
| NR_003051 | ENSG00000269900 | RMRP |
| NM_152470 | ENSG00000141622 | RNF165 |
| NM_001098638 | ENSG00000166439 | RNF169 |
| NR_046834 | ENSG00000237738 | RNF216-IT1 |
| NM_003958 | ENSG00000112130 | RNF8 |
| NR_023343 | ENSG00000264229 | RNU4ATAC |
| NR_125730 | ENSG00000207357 | RNU6-2 |
| NR_023344 | ENSG00000221676 | RNU6ATAC |
| NM_002941 | ENSG00000169855 | ROBO1 |
| NR_102746 | | ROPN1L-AS1 |
| NM_000975 | ENSG00000142676 | RPL11 |
| NM_002948 | ENSG00000174748 | RPL15 |
| NM_000983 | ENSG00000116251 | RPL22 |
| NM_000991 | ENSG00000108107 | RPL28 |
| NM_000992 | ENSG00000162244 | RPL29 |
| NM_000993 | ENSG00000071082 | RPL31 |
| NM_007209 | ENSG00000136942 | RPL35 |
| NM_015414 | ENSG00000130255 | RPL36 |
| NM_000998 | | RPL37A |
| NM_000999 | ENSG00000172809 | RPL38 |
| NM_021104 | ENSG00000229117 | RPL41 |
| NM_001003 | ENSG00000137818 | RPLP1 |
| NM_002950 | ENSG00000163902 | RPN1 |
| NR_002312 | ENSG00000277209 | RPPH1 |
| NM_015203 | ENSG00000163125 | RPRD2 |
| NM_005617 | ENSG00000164587 | RPS14 |
| NR_077246 | | RPS14P3 |
| NM_001019 | ENSG00000134419 | RPS15A |
| NM_001020 | ENSG00000105193 | RPS16 |
| NM_001022 | | RPS19 |
| NM_001023 | | RPS20 |
| NM_001025 | ENSG00000186468 | RPS23 |
| NM_001026 | ENSG00000138326 | RPS24 |
| NM_001032 | ENSG00000213741 | RPS29 |
| NM_001010 | ENSG00000137154 | RPS6 |
| NM_021135 | ENSG00000071242 | RPS6KA2 |
| NM_020761 | ENSG00000141564 | RPTOR |
| NM_015056 | ENSG00000160208 | RRP1B |
| NM_033112 | ENSG00000124541 | RRP36 |
| NM_007008 | ENSG00000115310 | RTN4 |
| NM_012234 | | RYBP |
| NM_002958 | ENSG00000163785 | RYK |
| NM_005979 | ENSG00000189171 | S100A13 |
| NM_014363 | ENSG00000151835 | SACS |
| NM_005500 | ENSG00000142230 | SAE1 |
| NM_174920 | ENSG00000167100 | SAMD14 |
| NM_015265 | ENSG00000119042 | SATB2 |
| NM_030962 | ENSG00000133812 | SBF2 |
| NM_014963 | ENSG00000064932 | SBNO2 |
| NM_004719 | ENSG00000139218 | SCAF11 |
| NM_020706 | ENSG00000156304 | SCAF4 |
| NM_173690 | ENSG00000173611 | SCAT |
| NM_005505 | ENSG00000073060 | SCARB1 |
| NR_004387 | ENSG00000239002 | SCARNA10 |
| NR_003012 | ENSG00000251898 | SCARNA11 |
| NR_003010 | ENSG00000238795 | SCARNA12 |
| NR_003002 | ENSG00000252481 | SCARNA13 |
| NR_004388 | ENSG00000252712 | SCARNA14 |
| NR_003023 | ENSG00000270066 | SCARNA2 |
| NR_003004 | ENSG00000249784 | SCARNA22 |
| NR_003007 | ENSG00000251869 | SCARNA23 |
| NR_132762 | | SCARNA26A |
| NR_132767 | | SCARNA26B |
| NR_003005 | ENSG00000280466 | SCARNA4 |
| NR_003008 | ENSG00000252010 | SCARNA5 |
| NR_003001 | ENSG00000238741 | SCARNA7 |
| NR_002569 | ENSG00000254911 | SCARNA9 |
| NM_016510 | ENSG00000132330 | SCLY |
| NM_014654 | | SDC3 |
| NM_033280 | ENSG00000166562 | SEC11C |
| NM_004892 | | SEC22B |
| NM_004206 | ENSG00000093183 | SEC22C |
| NM_003262 | ENSG00000008952 | SEC62 |
| NM_007214 | ENSG00000025796 | SEC63 |
| NM_031216 | ENSG00000085415 | SEH1L |
| NM_020858 | ENSG00000137872 | SEMA6D |
| NM_021627 | | SENP2 |
| NM_015640 | ENSG00000142864 | SERBP1 |
| NM_014509 | ENSG00000183569 | SERHL2 |
| NM_014445 | ENSG00000120742 | SERPI |
| NM_004568 | ENSG00000124570 | SERPINB6 |
| NM_003011 | ENSG00000119335 | SET |
| NM_012271 | ENSG00000181555 | SETD2 |
| NM_032233 | ENSG00000183576 | SETD3 |
| NM_018187 | ENSG00000168137 | SETD5 |
| NM_030648 | ENSG00000145391 | SETD7 |
| NM_015046 | ENSG00000107290 | SETX |
| NM_178860 | ENSG00000063015 | SEZ6 |
| NM_031287 | ENSG00000169976 | SF3B5 |
| NM_001018039 | ENSG00000198879 | SFMBT2 |
| NM_005066 | ENSG00000116560 | SFPQ |
| NM_144579 | ENSG00000144040 | SFXN5 |
| NM_015503 | ENSG00000178188 | SH2B1 |
| NM_020979 | ENSG00000160999 | SH2B2 |
| NM_001103160 | ENSG00000189410 | SH2D5 |
| NM_020145 | ENSG00000148341 | SH3GLB2 |
| NR_038940 | ENSG00000280693 | SH3PXD2A-AS1 |
| NM_020870 | ENSG00000154447 | SH3RF1 |
| NM_000193 | | SHH |
| NM_175908 | ENSG00000187902 | SHISA7 |
| NM_005866 | ENSG00000147955 | SIGMAR1 |
| NM_015073 | ENSG00000105738 | SIPA1L3 |
| NM_006427 | ENSG00000184990 | SIVA1 |
| NM_006930 | | SKP1 |
| NM_006527 | ENSG00000163950 | SLBP |
| NM_024628 | ENSG00000221955 | SLC12A8 |
| NR_103743 | ENSG00000226419 | SLC16A1-AS1 |
| NM_003054 | ENSG00000165646 | SLC18A2 |
| NM_005628 | ENSG00000105281 | SLC1A5 |
| NM_178526 | ENSG00000181035 | SLC25A42 |
| NM_030674 | ENSG00000111371 | SLC38A1 |
| NM_173514 | ENSG00000177058 | SLC38A9 |
| NM_173596 | ENSG00000139540 | SLC39A5 |
| NM_017836 | ENSG00000114544 | SLC41A3 |
| NM_033102 | ENSG00000158715 | SLC45A3 |
| NM_152672 | ENSG00000163959 | SLC51A |
| NM_016615 | ENSG00000010379 | SLC6A13 |
| NM_032290 | ENSG00000133302 | SLF1 |
| NM_014720 | ENSG00000065613 | SLK |
| NM_003070 | ENSG00000080503 | SMARCA2 |
| NM_003072 | ENSG00000127616 | SMARCA4 |
| NM_003075 | ENSG00000139613 | SMARCC2 |
| NM_014837 | ENSG00000116698 | SMG7 |
| NM_001136503 | | SMIM24 |
| NM_001124767 | | SMIM4 |
| NM_005871 | | SMNDC1 |
| NM_020197 | ENSG00000143499 | SMYD2 |
| NM_022743 | ENSG00000185420 | SMYD3 |
| NM_014390 | ENSG00000197157 | SND1 |
| NM_007241 | | SNF8 |
| NR_117096 | ENSG00000267322 | SNHG22 |
| NR_132782 | | SNORA100 |
| NR_002954 | ENSG00000212464 | SNORA12 |
| NR_002922 | ENSG00000238363 | SNORA13 |
| NR_002956 | ENSG00000207181 | SNORA14B |
| NR_002975 | ENSG00000276161 | SNORA17B |
| NR_002576 | ENSG00000199293 | SNORA21 |
| NR_002962 | ENSG00000201998 | SNORA23 |
| NR_002964 | ENSG00000272533 | SNORA28 |
| NR_002966 | ENSG00000206755 | SNORA30 |
| NR_002967 | ENSG00000199477 | SNORA31 |
| NR_002969 | ENSG00000206948 | SNORA36A |
| NR_002970 | ENSG00000207233 | SNORA37 |
| NR_002977 | ENSG00000212607 | SNORA3B |
| NR_002978 | ENSG00000207493 | SNORA46 |
| NR_003014 | ENSG00000238961 | SNORA47 |
| NR_002980 | ENSG00000206952 | SNORA50A |
| NR_003015 | ENSG00000212443 | SNORA53 |
| NR_002982 | ENSG00000207008 | SNORA54 |
| NR_002983 | ENSG00000201457 | SNORA55 |
| NR_002984 | ENSG00000206693 | SNORA56 |
| NR_004390 | ENSG00000206597 | SNORA57 |
| NR_002985 | | SNORA58 |
| NR_003025 | ENSG00000239149 | SNORA59A |
| NR_002919 | ENSG00000206838 | SNORA5A |
| NR_002325 | ENSG00000206760 | SNORA6 |
| NR_002326 | ENSG00000207405 | SNORA64 |
| NR_000012 | ENSG00000207166 | SNORA68 |
| NR_002910 | ENSG00000235408 | SNORA71B |
| NR_004404 | | SNORA73B |
| NR_002915 | ENSG00000200959 | SNORA74A |
| NR_002921 | ENSG00000206885 | SNORA75 |
| NR_002996 | ENSG00000200792 | SNORA80A |
| NR_028374 | ENSG00000206633 | SNORA80B |
| NR_132769 | | SNORA87 |
| NR_002952 | ENSG00000277184 | SNORA9 |
| NR_132772 | | SNORA90 |
| NR_132774 | | SNORA92 |
| NR_132778 | | SNORA98 |
| NR_003066 | | SNORD103C |
| NR_003079 | ENSG00000221066 | SNORD111 |
| NR_003030 | ENSG00000212304 | SNORD12 |
| NR_003685 | ENSG00000238886 | SNORD121A |
| NR_102369 | ENSG00000238793 | SNORD124 |
| NR_003693 | | SNORD126 |
| NR_132752 | | SNORD128 |
| NR_132972 | | SNORD129 |
| NR_132756 | | SNORD135 |
| NR_003045 | ENSG00000212232 | SNORD17 |
| NR_002441 | ENSG00000200623 | SNORD18A |
| NR_000008 | ENSG00000277194 | SNORD22 |
| NR_002602 | ENSG00000206775 | SNORD37 |
| NR_002751 | ENSG00000209702 | SNORD41 |
| NR_000013 | ENSG00000238423 | SNORD42B |
| NR_002439 | ENSG00000263764 | SNORD43 |
| NR_002741 | ENSG00000265145 | SNORD53 |
| NR_002738 | ENSG00000226572 | SNORD57 |
| NR_002736 | ENSG00000206630 | SNORD60 |
| NR_002913 | ENSG00000206989 | SNORD63 |
| NR_003054 | ENSG00000277512 | SNORD65 |
| NR_003055 | ENSG00000212158 | SNORD66 |
| NR_002450 | | SNORD68 |
| NR_003057 | ENSG00000212452 | SNORD69 |
| NR_000007 | ENSG00000208797 | SNORD73A |
| NR_002579 | | SNORD74 |
| NR_004398 | ENSG00000202400 | SNORD82 |
| NR_002598 | ENSG00000254341 | SNORD87 |
| NR_003073 | ENSG00000275084 | SNORD91B |
| NR_003074 | ENSG00000264994 | SNORD92 |
| NR_004378 | ENSG00000208772 | SNORD94 |
| NR_002592 | ENSG00000272296 | SNORD96A |
| NR_004379 | ENSG00000208883 | SNORD96B |
| NR_004403 | ENSG00000238622 | SNORD97 |
| NR_003076 | | SNORD98 |
| NR_003077 | ENSG00000221539 | SNORD99 |
| NM_014014 | ENSG00000144028 | SNRNP200 |
| NM_007020 | | SNRNP35 |
| NM_152551 | ENSG00000168566 | SNRNP48 |
| NM_003089 | ENSG00000104852 | SNRNP70 |
| NM_013322 | ENSG00000086300 | SNX10 |
| NM_020468 | ENSG00000135317 | SNX14 |
| NM_000454 | ENSG00000142168 | SOD1 |
| NM_080627 | ENSG00000149639 | SOGA1 |
| NM_006943 | ENSG00000177732 | SOX12 |
| NM_003111 | ENSG00000172845 | SP3 |
| NM_003116 | ENSG00000061656 | SPAG4 |
| NM_006461 | ENSG00000076382 | SPAG5 |
| NM_182513 | | SPC24 |
| NM_020675 | ENSG00000152253 | SPC25 |
| NM_012391 | ENSG00000124664 | SPDEF |
| NM_015001 | ENSG00000065526 | SPEN |
| NM_006542 | | SPHAR |
| NM_020126 | ENSG00000063176 | SPHK2 |
| NM_032566 | ENSG00000145879 | SPINK7 |
| NM_020148 | ENSG00000134278 | SPIRE1 |
| NM_139015 | ENSG00000157837 | SPPL3 |
| NM_181784 | ENSG00000198369 | SPRED2 |
| NM_025106 | ENSG00000171621 | SPSB1 |
| NM_003900 | ENSG00000161011 | SQSTM1 |
| NM_018079 | ENSG00000068784 | SRBD1 |
| NM_004599 | ENSG00000198911 | SREBF2 |
| NM_003131 | ENSG00000112658 | SRF |
| NM_003132 | ENSG00000116649 | SRM |
| NM_182691 | ENSG00000135250 | SRPK2 |
| NM_006924 | | SRSF1 |
| NM_003017 | | SRSF3 |
| NM_005626 | ENSG00000116350 | SRSF4 |
| NM_006275 | ENSG00000124193 | SRSF6 |
| NM_003144 | ENSG00000124783 | SSR1 |
| NM_003145 | ENSG00000163479 | SSR2 |
| NM_007107 | ENSG00000114850 | SSR3 |
| NM_014188 | ENSG00000160075 | SSU72 |
| NM_021978 | ENSG00000149418 | ST14 |
| NM_006100 | ENSG00000064225 | ST3GAL6 |
| NM_001037228 | | STARD7-AS1 |
| NM_007315 | ENSG00000115415 | STAT1 |
| NM_014393 | ENSG00000040341 | STAU2 |
| NM_004760 | ENSG00000164543 | STK17A |
| NM_003576 | ENSG00000102572 | STK24 |
| NM_030906 | ENSG00000130413 | STK33 |
| NM_005563 | ENSG00000117632 | STMN1 |
| NM_004099 | ENSG00000148175 | STOM |
| NM_153335 | ENSG00000266173 | STRADA |
| NM_018387 | ENSG00000165209 | STRBP |
| NM_003763 | ENSG00000124222 | STX16 |
| NM_005819 | ENSG00000135823 | STX6 |
| NM_022491 | ENSG00000111707 | SUDS3 |
| NM_014884 | ENSG00000064607 | SUGP2 |
| NM_015411 | ENSG00000129103 | SUMF2 |
| NM_025154 | ENSG00000164828 | SUN1 |
| NM_007192 | ENSG00000092201 | SUPT16H |
| NM_017503 | ENSG00000148291 | SURF2 |
| NM_006753 | | SURF6 |
| NM_153694 | ENSG00000139351 | SYCP3 |
| NM_004819 | ENSG00000125755 | SYMPK |
| NM_006372 | ENSG00000135316 | SYNCRIP |
| NM_015180 | ENSG00000054654 | SYNE2 |
| NM_032431 | ENSG00000162298 | SYVN1 |
| NM_004606 | ENSG00000147133 | TAF1 |
| NM_006284 | | TAF10 |
| NM_005643 | ENSG00000064995 | TAF11 |
| NM_005679 | ENSG00000103168 | TAF1C |
| NM_031923 | ENSG00000165632 | TAF3 |
| NM_005642 | ENSG00000178913 | TAF7 |
| NM_004783 | ENSG00000149930 | TAOK2 |
| NM_007375 | | TARDBP |
| NM_152295 | ENSG00000113407 | TARS |
| NM_025150 | ENSG00000143374 | TARS2 |
| NM_001097643 | | TAS2R30 |
| NM_020773 | ENSG00000132405 | TBC1D14 |
| NM_144628 | ENSG00000125875 | TBC1D20 |
| NM_014832 | ENSG00000136111 | TBC1D4 |
| NM_005993 | ENSG00000141556 | TBCD |
| NM_014726 | ENSG00000198933 | TBKBP1 |
| NM_005647 | ENSG00000101849 | TBL1X |
| NM_024665 | ENSG00000177565 | TBL1XR1 |
| NR_125749 | ENSG00000267280 | TBX2-AS1 |
| NM_005996 | ENSG00000135111 | TBX3 |
| NM_006706 | ENSG00000113649 | TCERG1 |
| NM_014972 | ENSG00000141002 | TCF25 |
| NM_003214 | ENSG00000007866 | TEAD3 |
| NR_001566 | ENSG00000270141 | TERC |
| NM_017746 | ENSG00000136891 | TEX10 |
| NM_018469 | ENSG00000136478 | TEX2 |
| NM_015926 | ENSG00000164081 | TEX264 |
| NR_033910 | | TFAP2A-AS1 |
| NM_178548 | ENSG00000116819 | TFAP2E |
| NM_014553 | ENSG00000115112 | TFCP2L1 |
| NM_003234 | ENSG00000072274 | TFRC |
| NM_003243 | ENSG00000069702 | TGFBR3 |
| NM_022065 | ENSG00000115970 | THADA |
| NM_138350 | ENSG00000041988 | THAP3 |
| NM_020449 | ENSG00000125676 | THOC2 |
| NM_024817 | ENSG00000187720 | THSD4 |
| NM_022037 | ENSG00000116001 | TIA1 |
| NM_003252 | | TIAL1 |
| NM_152259 | ENSG00000140534 | TICRR |
| NM_020375 | | TIGAR |
| NM_030953 | ENSG00000164296 | TIGD6 |
| NM_012458 | ENSG00000099800 | TIMM13 |
| NM_001001563 | ENSG00000105197 | TIMM50 |
| NM_153375 | ENSG00000223573 | TINCR |
| NM_004614 | ENSG00000166548 | TK2 |
| NM_001064 | ENSG00000163931 | TKT |
| NM_003260 | ENSG00000065717 | TLE2 |
| NM_012465 | ENSG00000095587 | TLL2 |
| NM_020123 | ENSG00000077147 | TM9SF3 |
| NM_003217 | ENSG00000139644 | TMBIM6 |
| NM_017905 | ENSG00000150403 | TMCO3 |
| NM_015348 | ENSG00000075568 | TMEM131 |
| NM_032928 | ENSG00000244187 | TMEM141 |
| NM_017814 | ENSG00000064545 | TMEM161A |
| NM_018475 | ENSG00000134851 | TMEM165 |
| NM_012264 | ENSG00000198792 | TMEM184B |
| NM_001003682 | ENSG00000253304 | TMEM200B |
| NM_016499 | ENSG00000187049 | TMEM216 |
| NM_001145529 | ENSG00000204278 | TMEM235 |
| NM_001114748 | | TMEM240 |
| NM_152261 | ENSG00000151135 | TMEM263 |
| NM_001256829 | ENSG00000080603 | TMEM265 |
| NM_018112 | ENSG00000095209 | TMEM38B |
| NM_014698 | ENSG00000196187 | TMEM63A |
| NM_016456 | ENSG00000116857 | TMEM9 |
| NM_014738 | ENSG00000177728 | TMEM94 |
| NM_020644 | ENSG00000175348 | TMEM9B |
| NR_027157 | ENSG00000257167 | TMPO-AS1 |
| NM_003840 | ENSG00000173530 | TNFRSF10D |
| NM_014452 | ENSG00000146072 | TNFRSF21 |
| NM_033396 | ENSG00000149115 | TNKS1BP1 |
| NM_025235 | ENSG00000107854 | TNKS2 |
| NM_001013722 | ENSG00000182095 | TNRC18 |
| NM_015319 | ENSG00000111077 | TNS2 |
| NM_016272 | ENSG00000183864 | TOB2 |
| NM_020243 | | TOMM22 |
| NM_001134493 | | TOMM6 |
| NM_003286 | ENSG00000198900 | TOP1 |
| NM_007027 | ENSG00000163781 | TOPBP1 |
| NM_022347 | ENSG00000169905 | TOR1AIP2 |
| NM_017723 | ENSG00000198113 | TOR4A |
| NM_000546 | ENSG00000141510 | TP53 |
| NM_017901 | ENSG00000186815 | TPCN1 |
| NM_005079 | ENSG00000076554 | TPD52 |
| NM_000365 | ENSG00000111669 | TPI1 |
| NM_000547 | ENSG00000115705 | TPO |
| NM_003292 | ENSG00000047410 | TPR |
| NM_004593 | | TRA2B |
| NM_003300 | ENSG00000131323 | TRAF3 |
| NR_034108 | ENSG00000231889 | TRAF3IP2-AS1 |
| NM_014965 | ENSG00000182606 | TRAK1 |
| NM_016292 | ENSG00000126602 | TRAP1 |
| NM_014408 | ENSG00000054116 | TRAPPC3 |
| NM_018415 | ENSG00000124496 | TRERF1 |
| NM_025195 | ENSG00000173334 | TRIB1 |
| NM_014818 | ENSG00000166436 | TRIM66 |
| NM_030912 | ENSG00000171206 | TRIM8 |
| NM_021820 | ENSG00000066651 | TRMT11 |
| NM_024950 | ENSG00000155275 | TRMT44 |
| NM_018006 | ENSG00000100416 | TRMU |
| NM_016000 | ENSG00000072756 | TRNT1 |
| NM_017636 | ENSG00000130529 | TRPM4 |
| NM_173485 | ENSG00000182463 | TSHZ2 |
| NR_028393 | ENSG00000270106 | TSNAX-DISC1 |
| NM_005724 | | TSPAN3 |
| NM_006675 | ENSG00000011105 | TSPAN9 |
| NR_002781 | ENSG00000235217 | TSPY26P |
| NM_003309 | | TSPYL1 |
| NM_022117 | ENSG00000184205 | TSPYL2 |
| NM_003310 | ENSG00000032389 | TSSC1 |
| NM_032037 | ENSG00000178093 | TSSK6 |
| NM_173500 | ENSG00000128881 | TTBK2 |
| NM_024525 | ENSG00000143643 | TTC13 |
| NM_001080441 | | TTC36 |
| NM_138376 | | TTC5 |
| NM_144596 | ENSG00000165533 | TTC8 |
| NM_015644 | | TTLL3 |
| NM_006082 | ENSG00000123416 | TUBA1B |
| NM_032704 | ENSG00000167553 | TUBA1C |
| NM_006088 | ENSG00000188229 | TUBB4B |
| NM_032525 | ENSG00000176014 | TUBB6 |
| NR_002323 | | TUG1 |
| NM_003322 | ENSG00000112041 | TULP1 |
| NM_020245 | ENSG00000130338 | TULP4 |
| NM_022830 | ENSG00000149016 | TUT1 |
| NM_175852 | ENSG00000084652 | TXLNA |
| NM_005499 | ENSG00000126261 | UBA2 |
| NM_016172 | ENSG00000130560 | UBAC1 |
| NM_177967 | ENSG00000134882 | UBAC2 |
| NM_018449 | ENSG00000137073 | UBAP2 |
| NM_021009 | ENSG00000150991 | UBC |
| NM_003343 | ENSG00000184787 | UBE2G2 |
| NM_005339 | ENSG00000078140 | UBE2K |
| NM_003969 | ENSG00000130725 | UBE2M |
| NM_021988 | ENSG00000244687 | UBE2V1 |
| NM_000462 | ENSG00000114062 | UBE3A |
| NM_198920 | ENSG00000118420 | UBE3D |
| NM_016936 | ENSG00000118900 | UBN1 |
| NM_172070 | ENSG00000144357 | UBR3 |
| NM_020765 | ENSG00000127481 | UBR4 |
| NM_015902 | ENSG00000104517 | UBR5 |
| NM_014233 | ENSG00000108312 | UBTF |
| NM_015562 | | UBXN7 |
| NM_031432 | ENSG00000130717 | UCK1 |
| NM_012474 | | UCK2 |
| NM_003355 | | UCP2 |
| NM_020120 | ENSG00000136731 | UGGT1 |
| NM_013282 | ENSG00000276043 | UHRF1 |
| NM_017979 | ENSG00000140553 | UNC45A |
| NM_001080419 | ENSG00000132478 | UNK |
| NM_006830 | ENSG00000127540 | UQCR11 |
| NM_003715 | ENSG00000138768 | USO1 |
| NM_005153 | ENSG00000103194 | USP10 |
| NR_046547 | | USP12-AS1 |
| NM_020718 | ENSG00000103404 | USP31 |
| NM_032582 | | USP32 |
| NM_014709 | ENSG00000115464 | USP34 |
| NM_025090 | ENSG00000055483 | USP36 |
| NR_038408 | | UST-AS1 |
| NM_006649 | ENSG00000156697 | UTP14A |
| NM_020368 | ENSG00000132467 | UTP3 |
| NM_003373 | ENSG00000035403 | VCL |
| NM_001001888 | ENSG00000205642 | VCX3B |
| NM_014667 | ENSG00000144560 | VGLL4 |
| NR_108060 | ENSG00000229124 | VIM-AS1 |
| NM_018445 | ENSG00000131871 | VIMP |
| NM_030938 | ENSG00000062716 | VMP1 |
| NM_173858 | | VN1R5 |
| NM_015378 | ENSG00000048707 | VPS13D |
| NM_022916 | | VPS33A |
| NM_015303 | ENSG00000156931 | VPS8 |
| NM_003384 | ENSG00000100749 | VRK1 |
| NR_026703 | ENSG00000199990 | VTRNA1-1 |
| NM_152718 | ENSG00000167992 | VWCE |
| NM_015045 | ENSG00000062650 | WAPL |
| NM_017883 | ENSG00000101940 | WDR13 |
| NM_144574 | ENSG00000140153 | WDR20 |
| NM_025160 | ENSG00000162923 | WDR26 |
| NM_182552 | ENSG00000184465 | WDR27 |
| NM_006784 | | WDR3 |
| NM_052844 | ENSG00000119333 | WDR34 |
| NM_018669 | ENSG00000160193 | WDR4 |
| NM_018268 | ENSG00000164253 | WDR41 |
| NM_019613 | ENSG00000141580 | WDR45B |
| NM_032118 | ENSG00000005448 | WDR54 |
| NM_007331 | ENSG00000109685 | WHSC1 |
| NM_017778 | ENSG00000147548 | WHSC1L1 |
| NM_015610 | ENSG00000157954 | WIPI2 |
| NM_004626 | ENSG00000085741 | WNT11 |
| NM_030753 | ENSG00000108379 | WNT3 |
| NR_126473 | ENSG00000251128 | WWC2-AS1 |
| NR_001564 | ENSG00000229807 | XIST |
| NM_003400 | ENSG00000082898 | XPO1 |
| NM_015171 | ENSG00000169180 | XPO6 |
| NM_001127438 | | XRCC6P5 |
| NM_003651 | ENSG00000060138 | YBX3 |
| NM_006555 | ENSG00000106636 | YKT6 |
| NM_014263 | ENSG00000136758 | YME1L1 |
| NM_006761 | ENSG00000108953 | YWHAE |
| NM_003406 | ENSG00000164924 | YWHAZ |
| NM_180990 | ENSG00000186919 | ZACN |
| NM_175907 | | ZADH2 |
| NM_001079 | ENSG00000115085 | ZAP70 |
| NM_003443 | ENSG00000116809 | ZBTB17 |
| NM_145166 | | ZBTB47 |
| NM_015898 | ENSG00000178951 | ZBTB7A |
| NM_024824 | ENSG00000100722 | ZC3H14 |
| NM_018471 | ENSG00000065548 | ZC3H15 |
| NM_021943 | ENSG00000156639 | ZFAND3 |
| NR_002438 | ENSG00000248492 | ZFAT-AS1 |
| NM_006885 | ENSG00000140836 | ZFHX3 |
| NM_004926 | ENSG00000185650 | ZFP36L1 |
| NM_133458 | ENSG00000184939 | ZFP90 |
| NR_125796 | | ZFPM2-AS1 |
| NM_003410 | ENSG00000005889 | ZFX |
| NM_015346 | ENSG00000072121 | ZFYVE26 |
| NM_144588 | ENSG00000155256 | ZFYVE27 |
| NM_003439 | ENSG00000106261 | ZKSCAN1 |
| NM_006956 | ENSG00000164631 | ZNF12 |
| NM_003434 | ENSG00000125846 | ZNF133 |
| NM_007152 | ENSG00000005801 | ZNF195 |
| NM_003455 | ENSG00000166261 | ZNF202 |
| NM_152287 | ENSG00000158805 | ZNF276 |
| NM_003575 | ENSG00000170265 | ZNF282 |
| NM_003421 | ENSG00000075407 | ZNF37A |
| NM_017757 | ENSG00000215421 | ZNF407 |
| NM_181489 | ENSG00000185219 | ZNF445 |
| NM_133464 | ENSG00000173258 | ZNF483 |
| NM_014930 | ENSG00000081386 | ZNF510 |
| NM_145806 | | ZNF511 |
| NM_152909 | ENSG00000188785 | ZNF548 |
| NM_024341 | ENSG00000130544 | ZNF557 |
| NM_152477 | ENSG00000196357 | ZNF565 |
| NM_152600 | | ZNF579 |
| NM_017652 | ENSG00000083828 | ZNF586 |
| NM_032828 | ENSG00000198466 | ZNF587 |
| NM_173539 | ENSG00000172748 | ZNF596 |
| NM_015042 | ENSG00000180357 | ZNF609 |
| NM_014497 | ENSG00000075292 | ZNF638 |
| NM_016620 | ENSG00000122482 | ZNF644 |
| NM_017865 | ENSG00000171163 | ZNF692 |
| NM_025069 | ENSG00000183779 | ZNF703 |
| NM_152557 | ENSG00000181220 | ZNF746 |
| NM_024702 | ENSG00000141579 | ZNF750 |
| NM_024910 | ENSG00000133624 | ZNF767P |
| NM_001137674 | ENSG00000197385 | ZNF860 |
| NM_080603 | ENSG00000168612 | ZSWIM1 |
| NM_020928 | ENSG00000130449 | ZSWIM6 |
| NM_001042697 | ENSG00000214941 | ZSWIM7 |
| NM_025112 | ENSG00000070476 | ZXDC |
| NM_015534 | ENSG00000036549 | ZZZ3 |

### REFERENCES

Aranda, S., Mas, G., and Di Croce, L. (2015). Regulation of gene transcription by Polycomb proteins. Sci Adv 1, e1500737.

Badis, G., Berger, M. F., Philippakis, A. A., Talukder, S., Gehrke, A. R., Jaeger, S. A., Chan, E. T., Metzler, G., Vedenko, A., Chen, X., et al. (2009). Diversity and complexity in DNA recognition by transcription factors. Science 324, 1720-1723.

Bag, J., and Bhattacharjee, R. B. (2010). Multiple levels of post-transcriptional control of expression of the poly (A)-binding protein. RNA Biol 7, 5-12.

Bailey, T. L., Boden, M., Buske, F. A., Frith, M., Grant, C. E., Clementi, L., Ren, J., Li, W. W., and Noble, W. S. (2009). MEME SUITE: tools for motif discovery and searching. Nucleic acids research 37, W202-208.

Beltran, M., Yates, C. M., Skalska, L., Dawson, M., Reis, F. P., Viiri, K., Fisher, C. L., Sibley, C. R., Foster, B. M., Bartke, T., et al. (2016). The interaction of PRC2 with RNA or chromatin is mutually antagonistic. Genome Res 26, 896-907.

Bernstein, E., Duncan, E. M., Masui, O., Gil, J., Heard, E., and Allis, C. D. (2006). Mouse polycomb proteins bind differentially to methylated histone H3 and RNA and are enriched in facultative heterochromatin. Molecular and cellular biology 26, 2560-2569.

Blackledge, N. P., Rose, N. R., and Klose, R. J. (2015). Targeting Polycomb systems to regulate gene expression: modifications to a complex story. Nat Rev Mol Cell Biol 16, 643-649.

Cheng, B., Ren, X., and Kerppola, T. K. (2014). KAP1 represses differentiation-inducible genes in embryonic stem cells through cooperative binding with PRC1 and derepresses pluripotency-associated genes. Mol Cell Biol 34, 2075-2091.

Feng, X., Grossman, R., and Stein, L. (2011). PeakRanger: a cloud-enabled peak caller for ChIP-seq data. BMC bioinformatics 12, 139.

Frith, M. C., Fu, Y., Yu, L., Chen, J. F., Hansen, U., and Weng, Z. (2004). Detection of functional DNA motifs via statistical over-representation. Nucleic acids research 32, 1372-1381.

Giresi, P. G., Kim, J., McDaniell, R. M., Iyer, V. R., and Lieb, J. D. (2007). FAIRE (Formaldehyde-Assisted Isolation of Regulatory Elements) isolates active regulatory elements from human chromatin. Genome Res 17, 877-885.

Glisovic, T., Bachorik, J. L., Yong, J., and Dreyfuss, G. (2008). RNA-binding proteins and post-transcriptional gene regulation. FEBS Lett 582, 1977-1986.

Grau, D. J., Chapman, B. A., Garlick, J. D., Borowsky, M., Francis, N. J., and Kingston, R. E. (2011). Compaction of chromatin by diverse Polycomb group proteins requires localized regions of high charge. Genes & development 25, 2210-2221.

Hendrickson, D., Kelley, D. R., Tenen, D., Bernstein, B., and Rinn, J. L. (2016). Widespread RNA binding by chromatin-associated proteins. Genome Biol 17, 28.

Incarnato, D., Neri, F., Anselmi, F., and Oliviero, S. (2014). Genome-wide profiling of mouse RNA secondary structures reveals key features of the mammalian transcriptome. Genome Biol 15, 491.

Jeon, Y., and Lee, J. T. (2011). YY1 tethers Xist RNA to the inactive X nucleation center. Cell 146, 119-133.

Ji, X., Li, W., Song, J., Wei, L., and Liu, X. S. (2006). CEAS: cis-regulatory element annotation system. Nucleic acids research 34, W551-554.

Kaneko, S., Bonasio, R., Saldana-Meyer, R., Yoshida, T., Son, J., Nishino, K., Umezawa, A., and Reinberg, D. (2014a). Interactions between JARID2 and noncoding RNAs regulate PRC2 recruitment to chromatin. Molecular cell 53, 290-300.

Kaneko, S., Son, J., Bonasio, R., Shen, S. S., and Reinberg, D. (2014b). Nascent RNA interaction keeps PRC2 activity poised and in check. Genes & development 28, 1983-1988.

Kaneko, S., Son, J., Shen, S. S., Reinberg, D., and Bonasio, R. (2013). PRC2 binds active promoters and contacts nascent RNAs in embryonic stem cells. Nature structural & molecular biology.

Kent, W. J., Sugnet, C. W., Furey, T. S., Roskin, K. M., Pringle, T. H., Zahler, A. M., and Haussler, D. (2002). The human genome browser at UCSC. Genome Res 12, 996-1006.

Khalil, A. M., Guttman, M., Huarte, M., Garber, M., Raj, A., Rivea Morales, D., Thomas, K., Presser, A., Bernstein, B. E., van Oudenaarden, A., et al. (2009). Many human large intergenic noncoding RNAs associate with chromatin-modifying complexes and affect gene expression. Proceedings of the National Academy of Sciences of the United States of America 106, 11667-11672.

Kim, J., Cantor, A. B., Orkin, S. H., and Wang, J. (2009). Use of in vivo biotinylation to study protein-protein and protein-DNA interactions in mouse embryonic stem cells. Nat Protoc 4, 506-517.

Kung, J. T., Kesner, B., An, J. Y., Ahn, J. Y., Cifuentes-Rojas, C., Colognori, D., Jeon, Y., Szanto, A., del Rosario, B. C., Pinter, S. F., et al. (2015). Locus-specific targeting to the X chromosome revealed by the RNA interactome of CTCF. Molecular cell 57, 361-375.

Lee, J. T., and Lu, N. (1999). Targeted mutagenesis of Tsix leads to nonrandom X inactivation. Cell 99, 47-57.

Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.

Ma, W., Noble, W. S., and Bailey, T. L. (2014). Motif-based analysis of large nucleotide data sets using MEME-ChIP. Nat Protoc 9, 1428-1450.

Magistri, M., Faghihi, M. A., St Laurent, G., 3rd, and Wahlestedt, C. (2012). Regulation of chromatin structure by long noncoding RNAs: focus on natural antisense transcripts. Trends in genetics : TIG 28, 389-396.

Mahony, S., and Benos, P. V. (2007). STAMP: a web tool for exploring DNA-binding motif similarities. Nucleic acids research 35, W253-258.

Marchese, D., de Groot, N. S., Lorenzo Gotor, N., Livi, C. M., and Tartaglia, G. G. (2016). Advances in the characterization of RNA-binding proteins. Wiley interdisciplinary reviews. RNA 7, 793-810.

Morey, L., Pascual, G., Cozzuto, L., Roma, G., Wutz, A., Benitah, S. A., and Di Croce, L. (2012). Nonoverlapping functions of the Polycomb group Cbx family of proteins in embryonic stem cells. Cell stem cell 10, 47-62.

Nicol, J. W., Helt, G. A., Blanchard, S. G., Jr., Raja, A., and Loraine, A. E. (2009). The Integrated Genome Browser: free software for distribution and exploration of genome-scale datasets. Bioinformatics 25, 2730-2731.

O'Loghlen, A., Munoz-Cabello, A. M., Gaspar-Maia, A., Wu, H. A., Banito, A., Kunowska, N., Racek, T., Pemberton, H. N., Beolchi, P., Lavial, F., et al. (2012). MicroRNA regulation of Cbx7 mediates a switch of Polycomb orthologs during ESC differentiation. Cell stem cell 10, 33-46.

Pinter, S. F., Sadreyev, R. I., Yildirim, E., Jeon, Y., Ohsumi, T. K., Borowsky, M., and Lee, J. T. (2012). Spreading of X chromosome inactivation via a hierarchy of defined Polycomb stations. Genome Res 22, 1864-1876.

Quinlan, A. R., and Hall, I. M. (2010). BEDTools: a flexible suite of utilities for comparing genomic features. Bioinformatics 26, 841-842.

Ramirez, F., Dundar, F., Diehl, S., Gruning, B. A., and Manke, T. (2014). deepTools: a flexible platform for exploring deep-sequencing data. Nucleic acids research 42, W187-191.

Ray, D., Kazan, H., Cook, K. B., Weirauch, M. T., Najafabadi, H. S., Li, X., Gueroussov, S., Albu, M., Zheng, H., Yang, A., et al. (2013). A compendium of RNA-binding motifs for decoding gene regulation. Nature 499, 172-177.

Ray, M. K., Wiskow, O., King, M.J., Ismail, N., Ergun, A., Wang, Y., Plys, A. J., Davis, C. P., Kathrein, K., Sadreyev, R., et al. (2016). CAT7 and cat71 long non-coding RNAs Tune Polycomb Repressive Complex 1 Function During Human and Zebrafish Development. J Biol Chem.

Rouskin, S., Zubradt, M., Washietl, S., Kellis, M., and Weissman, J. S. (2014). Genome-wide probing of RNA structure reveals active unfolding of mRNA structures in vivo. Nature 505, 701-705.

Rozowsky, J., Euskirchen, G., Auerbach, R. K., Zhang, Z. D., Gibson, T., Bjornson, R., Carriero, N., Snyder, M., and Gerstein, M. B. (2009). PeakSeq enables systematic scoring of ChIP-seq experiments relative to controls. Nat Biotechnol 27, 66-75.

Sandelin, A., Alkema, W., Engstrom, P., Wasserman, W. W., and Lenhard, B. (2004). JASPAR: an open-access database for eukaryotic transcription factor binding profiles. Nucleic acids research 32, D91-94.

Sarma, K., Levasseur, P., Aristarkhov, A., and Lee, J. T. (2010). Locked nucleic acids (LNAs) reveal sequence requirements and kinetics of Xist RNA localization to the X chromosome. Proc Natl Acad Sci USA 107, 22196-22201.

Shin, H., Liu, T., Manrai, A. K., and Liu, X. S. (2009). CEAS: cis-regulatory element annotation system. Bioinformatics 25, 2605-2606.

Sigova, A. A., Abraham, B. J., Ji, X., Molinie, B., Hannett, N. M., Guo, Y. E., Jangi, M., Giallourakis, C. C., Sharp, P. A., and Young, R. A. (2015). Transcription factor trapping by RNA in gene regulatory elements. Science 350, 978-981.

Simon, J. A., and Kingston, R. E. (2013). Occupying chromatin: Polycomb mechanisms for getting to genomic targets, stopping transcriptional traffic, and staying put. Molecular cell 49, 808-824.

Simon, J. M., Giresi, P. G., Davis, I. J., and Lieb, J. D. (2012). Using formaldehyde-assisted isolation of regulatory elements (FAIRE) to isolate active regulatory DNA. Nat Protoc 7, 256-267.

Spassov, D. S., and Jurecic, R. (2003). The PUF family of RNA-binding proteins: does evolutionarily conserved structure equal conserved function? IUBMB Life 55, 359-366.

Spitale, R. C., Flynn, R. A., Zhang, Q. C., Crisalli, P., Lee, B., Jung, J. W., Kuchelmeister, H. Y., Batista, P. J., Torre, E. A., Kool, E. T., et al. (2015). Structural imprints in vivo decode RNA regulatory mechanisms. Nature 519, 486-490.

Taliaferro, J. M., Lambert, N. J., Sudmant, P. H., Dominguez, D., Merkin, J. J., Alexis, M. S., Bazile, C., and Burge, C. B. (2016). RNA Sequence Context Effects Measured In Vitro Predict In Vivo Protein Binding and Regulation. Molecular cell 64, 294-306.

Tamura, K., Stecher, G., Peterson, D., Filipski, A., and Kumar, S. (2013). MEGA6: Molecular Evolutionary Genetics Analysis version 6.0. Mol Biol Evol 30, 2725-2729.

Tavares, L., Dimitrova, E., Oxley, D., Webster, J., Poot, R., Demmers, J., Berstarosti, K., Taylor, S., Ura, H., Koide, H., et al. (2012). RYBP-PRC1 complexes mediate H2A ubiquitylation at polycomb target sites independently of PRC2 and H3K27 me3. Cell 148, 664-678.

Trapnell, C., Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nat Protoc 7, 562-578.

Van Nostrand, E. L., Pratt, G. A., Shishkin, A. A., Gelboin-Burkhart, C., Fang, M. Y., Sundararaman, B., Blue, S. M., Nguyen, T. B., Surka, C., Elkins, K., et al. (2016). Robust transcriptome-wide discovery of RNA-binding protein binding sites with enhanced CLIP (eCLIP). Nat Methods 13, 508-514.

Vierstra, J., Rynes, E., Sandstrom, R., Zhang, M., Canfield, T., Hansen, R. S., Stehling-Sun, S., Sabo, P. J., Byron, R., Humbert, R., et al. (2014). Mouse regulatory DNA landscapes reveal global principles of cis-regulatory evolution. Science 346, 1007-1012.

Wang, J., and Bell, L. R. (1994). The Sex-lethal amino terminus mediates cooperative interactions in RNA binding and is essential for splicing regulation. Genes & development 8, 2072-2085.

Wang, X., Goodrich, K. J., Gooding, A. R., Naeem, H., Archer, S., Paucek, R. D., Youmans, D. T., Cech, T. R., and Davidovich, C. (2017). Targeting of Polycomb Repressive Complex 2 to RNA by Short Repeats of Consecutive Guanines. Molecular cell 65, 1056-1067 e1055.

Warzecha, C. C., Sato, T. K., Nabet, B., Hogenesch, J. B., and Carstens, R. P. (2009). ESRP1 and ESRP2 are epithelial cell-type-specific regulators of FGFR2 splicing. Molecular cell 33, 591-601.

Wei, G. H., Badis, G., Berger, M. F., Kivioja, T., Palin, K., Enge, M., Bonke, M., Jolma, A., Varjosalo, M., Gehrke, A. R., et al. (2010). Genome-wide analysis of ETS-family DNA-binding in vitro and in vivo. EMBO J 29, 2147-2160.

Woo, C. J., Maier, V. K., Davey, R., Brennan, J., Li, G., Brothers, J., 2nd, Schwartz, B., Gordo, S., Kasper, A., Okamoto, T. R., et al. (2017). Gene activation of SMN by selective disruption of lncRNA-mediated recruitment of PRC2 for the treatment of spinal muscular atrophy. Proc Natl Acad Sci USA 114, E1509-E1518.

Xie, Z., Hu, S., Blackshaw, S., Zhu, H., and Qian, J. (2010). hPDI: a database of experimental human protein-DNA interactions. Bioinformatics 26, 287-289.

Yap, K. L., Li, S., Munoz-Cabello, A. M., Raguz, S., Zeng, L., Mujtaba, S., Gil, J., Walsh, M. J., and Zhou, M. M. (2010). Molecular interplay of the noncoding RNA ANRIL and methylated histone H3 lysine 27 by polycomb CBX7 in transcriptional silencing of INK4a. Molecular cell 38, 662-674.

Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.

Zhao, J., Ohsumi, T. K., Kung, J. T., Ogawa, Y., Grau, D. J., Sarma, K., Song, J. J., Kingston, R. E., Borowsky, M., and Lee, J. T. (2010). Genome-wide identification of polycomb-associated RNAs by RIP-seq. Molecular cell 40, 939-953.

Zhen, C. Y., Tatavosian, R., Huynh, T. N., Duc, H. N., Das, R., Kokotovic, M., Grimm, J. B., Lavis, L. D., Lee, J., Mejia, F. J., et al. (2016). Live-cell single-molecule tracking reveals co-recognition of H3K27 me3 and DNA targets polycomb Cbx7-PRC1 to chromatin. Elife 5.

Zovoilis, A., Cifuentes-Rojas, C., Chu, H. P., Hernandez, A. J., and Lee, J. T. (2016). Destabilization of B2 RNA by EZH2 Activates the Stress Response. Cell 167, 1788-1802 e1713.
Chen, B., Yun, J., Kim, M. S., Mendell, J. T., and Xie, Y. (2014). PIPE-CLIP: a comprehensive online tool for CLIP-seq data analysis. Genome biology 15, R18.
Ray, M. K., Wiskow, O., King, M. J., Ismail, N., Ergun, A., Wang, Y., Plys, A. J., Davis, C. P., Kathrein, K., Sadreyev, R., et al. (2016). CAT7 and cat71 long non-coding RNAs Tune Polycomb Repressive Complex 1 Function During Human and Zebrafish Development. The Journal of biological chemistry.
Spitale, R. C., Flynn, R. A., Zhang, Q. C., Crisalli, P., Lee, B., Jung, J. W., Kuchelmeister, H. Y., Batista, P. J., Torre, E. A., Kool, E. T., et al. (2015). Structural imprints in vivo decode RNA regulatory mechanisms. Nature 519, 486-490.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A process of preparing an inhibitory nucleic acid that specifically binds, or is complementary to, a region of an RNA comprising a motif as shown in TABLE 1, wherein the RNA is known to bind to Polycomb repressive complex 1 (PRC1), selected from the group consisting of SEQ ID NOs:1 to 5893 (human), 5894 to 17415 (human), and 17416 to 36368 (mouse), the process comprising the step of designing and/or synthesizing an inhibitory nucleic acid of between 5 and 40 bases in length, that specifically binds to a region of the RNA that binds PRC1.

2. The process of claim 1, wherein the sequence of the designed and/or synthesized inhibitory nucleic acid is a nucleic acid sequence that is complementary to said region comprising a motif as described herein sequence that binds to PRC1, or is complementary to a portion thereof, said portion having a length of from 5 to 40 contiguous base pairs.

3. The process of claim 1, wherein the inhibitory nucleic acid modulates expression of a gene and the region of the RNA comprising the motif as described herein can be in 3′UTR, 5′UTR, coding region, or introns of a coding gene.

4. An inhibitory nucleic acid of about 10 to 50 bases in length that specifically binds, or is complementary to, a fragment of at least seven consecutive bases comprising a motif as shown in TABLE 1 within any of SEQ ID NOs:1 to 5893 (human) or 5894 to 17415 (human) or 17416 to 36368 (mouse), wherein the inhibitory nucleic acid comprises one or more modifications and modulates expression of a gene targeted by the RNA.

5. A composition comprising the inhibitory nucleic acid of claim 4.

6. The composition of claim 5, which is for parenteral administration.

7. The composition of claim 5, wherein the RNA sequence is in the 3′UTR of a gene, and the inhibitory nucleic acid is capable of upregulating expression of a gene targeted by the RNA.

8. A method of modulating gene expression in a cell or a mammal comprising administering to the cell or the mammal the composition of claim 5.

9. The inhibitory nucleic acid of claim 4, wherein the inhibitory nucleic acid comprises one or more modifications comprising: a modified sugar moiety, a modified internucleoside linkage, a modified nucleotide and/or combinations thereof.

10. The inhibitory nucleic acid of claim 4, wherein the inhibitory nucleic acid is an antisense oligonucleotide, LNA molecule, PNA molecule, ribozyme or siRNA.

11. The inhibitory nucleic acid of claim 4, wherein the inhibitory nucleic acid is double stranded and comprises an overhang at one or both termini.

12. The inhibitory nucleic acid of claim 4, wherein the inhibitory nucleic acid is a single- or double-stranded RNA interference (RNAi) compound.

13. The inhibitory nucleic acid of claim 4, wherein the RNAi compound is selected from the group consisting of short interfering RNA (siRNA); or a short, hairpin RNA (shRNA); small RNA-induced gene activation (RNAa); and small activating RNAs (saRNAs).

14. The inhibitory nucleic acid of claim 9, wherein the modified internucleoside linkage comprises at least one of: alkylphosphonate, phosphorothioate, phosphorodithioate, alkylphosphonothioate, phosphoramidate, carbamate, carbonate, phosphate triester, acetamidate, carboxymethyl ester, or combinations thereof.

15. The inhibitory nucleic acid of claim 9, wherein the modified sugar moiety comprises a 2′-O-methoxyethyl modified sugar moiety, a 2′-methoxy modified sugar moiety, a 2′-O-alkyl modified sugar moiety, or a bicyclic sugar moiety.

16. The inhibitory nucleic acid of claim 9, comprising: 2′-OMe, 2′-F, LNA, PNA, FANA, ENA or morpholino modifications.

17. A method for treating a subject with MECP2 Duplication Syndrome, the method comprising administering a therapeutically effective amount of an inhibitory nucleic acid targeting a PRC1-binding region comprising a motif as shown in TABLE 1 in Mecp2 RNA, preferably wherein the PRC1 binding region comprises SEQ ID NO:5876 or 5877.

18. The method of claim 17, comprising administering an inhibitory nucleic acid targeting a sequence comprising a motif as shown in TABLE 1 within the 3′UTR of Mecp2.

19. A method for treating a subject with systemic lupus erythematosis, the method comprising administering a therapeutically effective amount of an inhibitory nucleic acid targeting a PRC1-binding region comprising a motif as shown in TABLE 1 in IRAK1 RNA, preferably wherein the PRC1 binding region comprises SEQ ID NO:5874 or 5875.

20. The method of claim 19, comprising administering an inhibitory nucleic acid targeting a sequence comprising a motif as described herein within the 3′UTR of IRAK1.

21. The method of any claim 17, wherein the inhibitory nucleic acid comprises at least one locked nucleotide (LNA).

22. The method of any claim 19, wherein the inhibitory nucleic acid comprises at least one locked nucleotide (LNA).
